(19) United States
(12) Patent Application Publication
(10) Pub. No.: US 2024/0383962 A1
(43) Pub. Date: Nov. 21, 2024

(54) GENETICALLY ENGINEERED CELLS AND USES THEREOF

(71) Applicant: Cure Genetics Co., Limited, Suzhou (CN)

(72) Inventors: Zhuo Huang, Suzhou (CN); Yanni Lin, Suzhou (CN); Xiaocui Zheng, Suzhou (CN); Hongmei Kong, Suzhou (CN); Wenbo Wang, Suzhou (CN); Aihua Feng, Suzhou (CN)

(21) Appl. No.: 17/634,963

(22) PCT Filed: Aug. 10, 2020
(86) PCT No.: PCT/CN2020/108116

§ 371 Date: Nov. 9, 2022

(30) Foreign Application Priority Data
Aug. 13, 2019 (CN) 201910746355.8
Dec. 10, 2019 (WO) PCT/CN2019/124321

(51) Int. Cl. C07K 14/74; A61K 39/00; C07K 14/725; C07K 16/28; C12N 5/0783

(52) U.S. Cl. CPC C07K 14/70539; A61K 39/4611; A61K 39/4631; A61K 39/4632; A61K 39/464412; A61K 39/464417; A61K 39/464488; C07K 14/7051; C07K 16/2803; C07K 16/2878; C12N 5/0636; C07K 2317/73; C07K 2319/02; C12N 2510/00

(57) ABSTRACT

Disclosed herein are fusion proteins having a β2M polypeptide and a presenting peptide, and nucleic acids that encode such fusion proteins. Provided herein are also genetically engineered cells expressing such fusion protein, methods of their production, and their uses in allogeneic transplant. CAR-T cells expressing fusion protein disclosed herein and their uses in cancer treatment are also disclosed.

### 1. CROSS-REFERENCE

This application is a U.S. National Stage Application under 35 U.S.C. § 371 of International Application No. PCT/CN2020/108116, filed Aug. 10, 2020, which claims priority to Chinese Patent Application No. CN201910746355.8, filed Aug. 13, 2019, and International Patent Application No. PCT/CN2019/124321, filed Dec. 10, 2019, each of which is entirely incorporated herein by reference.

### 2. FIELD

The present invention relates to genetically engineered cells, and more particularly to engineered cells that are resistant to an allogeneic immune system, and further relates to methods for preparing the cells and uses thereof in allogeneic transplant.

### 3. BACKGROUND

Allograft rejection is the main cause of failure of allogeneic organ transplantation or cell transplantation. While T cells play essential roles in allograft rejection, recent studies have demonstrated unexpected roles for non-T cells such as natural killer (NK) cells, B cells, macrophage and mast cells in regulating transplant outcomes. (Li, Transplantation, 2010; 90(10): 1043-1047). For example, the unique self-non-self recognition system of the NK cells are highly relevant in allograft transplant. Briefly, individual NK cells have both stimulatory and inhibitory receptors on the cell surface, and signals from both types of receptors are required to establish NK tolerance to autologous cells. In humans, the inhibitory receptors include killer-cell immunoglobulin-like receptors (KIRs). Additionally, NKG2A and CD94 usually form heterodimers on the cell surface and function as inhibitory receptors, in not only NK cells but also T cells. Binding of “self” Class I MHC to such inhibitory receptors inhibits NK cells, and prevents the NK cells from attacking “self” cells. In transplant models, NK cells in the recipients can readily recognize MHC incompatible allogeneic cells via “missing self” recognition, as allogeneic cells lack self Class I MHC to engage NK inhibitory receptors. The lack of inhibitory signals triggers NK activation, which includes cytolytic activities and production of potent pro-inflammatory cytokines. The same mechanism also applies in the activation of other immune cells, such as T cells, and results in allograft rejection.

The chimeric antigen receptor T cell (CAR-T) therapy has shown promising curative effect in cancers such as recurrent and refractory lymphoma, leukemia, multiple myeloma. However, the existing autologous CAR-T technology requires individualized preparation of cells, and is therefore limited by the long production cycle, high cost, and in many cases, lack of adequate T cells from patients. Therefore, universal CAR-T (UCAR-T) therapies, in which the T cells are derived from healthy donors and prepared in advance for use by any patient, have attracted great interests. However, the bidirectional rejection between the T cell transplant and the recipient, like in any other allograft transplantation, need to be addressed. The present disclosures address the needs to overcome allograft rejection in allogeneic organ transplantation and cell transplantation, and provide related advantages.

### 4. SUMMARY

Provided herein are fusion proteins comprising a presenting peptide covalently linked to a beta-2-microglobulin (β2M) peptide via a linker, wherein the fusion protein binds a major histocompatibility (MHC) heavy chain to form an MHC complex that binds an inhibitory receptor of an immune cell to inhibit the immune cell, and wherein the fusion protein (1) comprises less than 500 amino acids, or (2) lacks an HLA-E heavy chain.

The fusion proteins provided herein bind a major histocompatibility (MHC) heavy chain to form an MHC complex that binds an inhibitory receptor of an immune cell to inhibit the immune cell. In some embodiments, the inhibitory receptor of the immune cell is NKG2A. In some embodiments, the inhibitory receptor of the immune cell is selected from the group consisting of KIR2DL1, KIR2DL2, KIR2DL3, KIR2DL4, KIR2DL5, KIR3DL1, KIR3DL2, KIR3DL3, and LIR1.

In some embodiments, the MHC heavy chain is a classical Class I MHC heavy chain, a non-classical Class I MHC heavy chain, or an MHC-like heavy chain. In some embodiments, the MHC heavy chain is a classical Class I MHC heavy chain selected from the group consisting of an HLA-A heavy chain, an HLA-B heavy chain, and an HLA-C heavy chain. In some embodiments, the MHC heavy chain is a nonclassical Class I MHC heavy chain selected from the group consisting of an HLA-E heavy chain, an HLA-F heavy chain, and an HLA-G heavy chain. In some embodiments, the MHC heavy chain is an MHC-like molecule heavy chain selected from the group consisting of a CD1 heavy chain, an MR1 heavy chain, an FcRn heavy chain, and UL18. In some embodiments, the MHC heavy chain is an HLA-E heavy chain.

In some embodiments, provided herein are fusion proteins comprising a presenting peptide covalently linked to a β2M peptide via a linker, wherein the presenting peptide is an HLA-E-restricted presenting peptide, and wherein the fusion protein (1) comprises less than 500 amino acids, or (2) lacks an HLA-E heavy chain.

In some embodiments of the fusion proteins provided herein, the presenting peptide is derived from a virus, a prokaryote, a eukaryote, or a mammal. In some embodiments the presenting peptide is derived from a human.

In some embodiments of the fusion proteins provided herein, the presenting peptide is a signal peptide of a Class I MHC molecule or a fragment thereof.

In some embodiments, fusion proteins provided herein comprise a presenting peptide covalently linked to a β2M peptide via a linker, wherein the presenting peptide is a signal peptide of a Class I MHC molecule, or a fragment thereof, and wherein the fusion protein (1) comprises less than 500 amino acids, or (2) lacks an HLA-E heavy chain.

In some embodiments, the presenting peptide has 5-30 amino acids. In some embodiments, the presenting peptide has 7-20 amino acids. In some embodiments, the presenting peptide has 8-10 amino acids.

In some embodiments of the fusion proteins provided herein, the presenting peptide is a signal peptide of a Class I MHC molecule, or a fragment thereof, wherein the Class I MHC molecule is selected from the group consisting of the heavy chains of HLA-A1, HLA-A2, HLA-A*3401, HLA-A*80, HLA-B7, HLA-B*13, HLA-B15, HLA-Cw3, HLA-Cw*2, HLA-Cw*0809, HLA-Cw7, HLA-Cw*1701, HLA-G, and HLA-F.

In some embodiments, the amino acid sequence of the presenting peptide comprises X1X2X₃X4X₅X6X₇X8L; wherein X1 is V or I; X2 is T, A, M or L; X3 is A, P, K or N; X4 is P, L, or T; X5 is R, Q or K; X6 is T or A; X7 is L, I, V or P; X8 is V, L, I, F or T (SEQ ID NO:115). In some embodiments, the amino acid sequence of the presenting peptide is selected from the group consisting of SEQ ID NOs: 21-73. In some embodiments, the amino acid sequence of presenting peptide is VMAPRTVLL (SEQ ID NO: 38).

In some embodiments, the fusion proteins provided herein comprise less than 500, less than 400, less than 300, or less than 200 amino acids. In some embodiments, the fusion protein comprises about 120-180 amino acids.

In some embodiments, the fusion proteins provided herein comprise the presenting peptide, the linker, and the β2M peptide from N-terminus to C-terminus.

In some embodiments of the fusion proteins provided herein, the amino acid sequence of the linker comprises (EAAAK)n, wherein n=3, 4 or 5 (SEQ ID NO: 110), and wherein the linker has between 5 and 30 amino acids. In some embodiments, the amino acid sequence of the linker comprises (GGGGS)n, wherein n=3, 4 or 5 (SEQ ID NO: 112), and wherein the linker has between 5 and 30 amino acids. In some embodiments, the amino acid sequence of the linker is SEQ ID NO:1 or 2.

In some embodiments of the fusion proteins provided herein, the amino acid sequence of the β2M peptide is at least 85%, at least 90%, or at least 95% identical to SEQ ID NO:81. In some embodiments, the amino acid sequence of the β2M peptide is SEQ ID NO:81.

In some embodiments, the fusion proteins provided herein consist of the presenting peptide, the linker, and the β2M peptide.

In some embodiments, the amino acid sequence of the fusion proteins provided herein is at least 85%, at least 90%, or at least 95% identical an amino acid sequence selected from the group consisting of SEQ ID NOs: 5 and 13-18. In some embodiments, the amino acid sequence of the fusion protein is selected from the group consisting of SEQ ID NOs: 5 and 13-18.

Provided herein are also nucleic acids that encode the fusion proteins provided herein.

In some embodiments, provided herein are nucleic acids comprising (i) a first fragment encoding a fusion protein comprising a presenting peptide and a β2M peptide covalently linked via a linker, wherein the fusion protein (1) comprises less than 500 amino acids, or (2) lacks an HLA-E heavy chain; and (ii) a second fragment encoding a synthetic receptor.

In some embodiments, the synthetic receptor is selected from the group consisting of a chimeric antigen receptor (CAR), a T cell receptor (TCR), a TCR receptor fusion construct (TRuC), a T cell antigen coupler (TAC), an antibody TCR receptor (AbTCR) and a chimeric CD3 receptor. In some embodiments, the synthetic receptor is a CAR.

In some embodiments, the synthetic receptor comprises an antigen-binding domain that specifically binds a tumor antigen. In some embodiments, the tumor antigen is selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1. In some embodiments, the tumor antigen is CD19 or BCMA.

In some embodiments, the synthetic receptor comprises an antigen-binding domain that specifically binds a viral antigen. In some embodiments, the viral antigen is EBV or HPV.

In some embodiments of the nucleic acids provided herein, the first and second fragments are connected via a polynucleotide encoding a 2A peptide. In some embodiments, the 2A peptide is a P2A peptide, a T2A peptide, a F2A peptide, or an E2A peptide. In some embodiments, the nucleic acids provided herein encode the synthetic receptor, the 2A peptide, the fusion protein, from N-terminus to C-terminus.

In some embodiments of the nucleic acids provided herein, the first and second fragments are connected via an IRES sequence.

Provided herein are also vectors that comprise the nucleic acids disclosed herein.

In some embodiments, the vector is a viral vector. In some embodiments, the viral vector is a lentiviral vector, an adenoviral vector, or an adeno-associated viral vector.

Provided herein are also genetically engineered cells expressing the fusion proteins disclosed herein.

Provided herein are also genetically engineered cells comprising the nucleic acids disclosed herein, or the vectors disclosed herein.

In some embodiments, the cells provided herein further express a synthetic receptor.

In some embodiments, the cells provided herein further comprise a second nucleic acid that encodes a synthetic receptor. In some embodiments, the synthetic receptor is selected from the group consisting of a CAR, a TCR, a TRuC, a TAC, an AbTCR, and a chimeric CD3 receptor. In some embodiments, the synthetic receptor is a CAR.

In some embodiments, the synthetic receptor comprises an antigen-binding domain that specifically binds a tumor antigen. In some embodiments, the tumor antigen is selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1. In some embodiments, the tumor antigen is CD19 or BCMA.

In some embodiments, the synthetic receptor is a CAR having an antigen-binding domain that specifically binds CD19 or BCMA, wherein the amino acid sequence of the CAR is selected from the group consisting of SEQ ID NOs: 74-80 and 136.

In some embodiments, the synthetic receptor is a TCR having an antigen-binding domain that specifically binds NY-ESO-1, wherein the amino acid sequence of the TCR is SEQ ID NO:132.

In some embodiments, the synthetic receptor comprises an antigen-binding domain that specifically binds a viral antigen. In some embodiments, the viral antigen is EBV or HPV.

In some embodiments of the cells provided herein, the fusion proteins provided herein form a complex with an endogenous MHC heavy chain on the cell surface.

In some embodiments, the cells provided herein lack endogenous expression of at least one gene encoding a Class I MHC molecule or an MHC-like molecule on the cell surface. In some embodiments, the cells provided herein lack endogenous expression of a Class I MHC molecule selected from the group consisting of heavy chains of HLA-A, HLA-B, HLA-C, HLA-E, HLA-F, and HLA-G on the cell surface. In some embodiments, the cells provided herein lack endogenous expression of β2M on the cell surface. In some embodiments, the cells provided herein lack endogenous expression of an MHC-like molecule selected from the group consisting of a CD1 heavy chain, an MR1 heavy chain, an FcRn heavy chain, and UL18 on the cell surface.

In some embodiments, the cells provided herein are immune cells. In some embodiments, the cells provided herein are leukocytes. In some embodiments, the leukocyte is selected from the group consisting of a T cell, a NK cell, a NKT cell, a B cell, a plasma cell, a dendritic cell, a neutrophil, a monocyte, a macrophage, and a granulocyte. In some embodiments, the leukocyte is a NK cell. In some embodiments, the leukocyte is a T cell.

In some embodiments, at least one gene encoding a component of TCR complex is inactivated in the T cell.

Provided herein are also pharmaceutical compositions having the cells of provided herein and a pharmaceutically acceptable excipient.

Provided herein are also uses of the cells provided herein in an allogeneic transplant.

Provided herein are also uses of the cells provided herein in cancer treatment.

Provided herein are also uses of the cells provided herein for the preparation of a medicament for the treatment of cancer.

Provided herein are also uses of the cells provided herein in an allogeneic transplant, wherein the cells comprise a nucleic acid encoding a fusion protein comprising a presenting peptide and a β2M peptide covalently linked via a linker, and wherein the fusion protein (1) comprises less than 500 amino acids, or (2) lacks an HLA-E heavy chain.

Provided herein are also uses of the cells provided herein in an allogeneic transplant, wherein the cells express a fusion protein comprising a presenting peptide and a β2M peptide covalently linked via a linker, and wherein the fusion protein (1) comprises less than 500 amino acids, or (2) lacks an HLA-E heavy chain.

Provided herein are also methods of treating a subject in need of an allogeneic transplant, comprising administering an effective amount of the cells provided herein to the subject.

Provided herein are also methods of treating cancer in a subject comprising administering a therapeutically effective amount of the cells provided herein to the subject.

Provided herein are also methods of treating a subject in need of an allogeneic transplant, comprising administering an effective amount of an cell to the subject, wherein the cell comprises a nucleic acid encoding a fusion protein comprising a presenting peptide and a β2M peptide covalently linked via a linker, and wherein the fusion protein (1) comprises less than 500 amino acids, or (2) lacks an HLA-E heavy chain.

Provided herein are also methods of treating a subject in need of an allogeneic transplant, comprising administering an effective amount of an cell to the subject, wherein the cell expresses a fusion protein comprising a presenting peptide and a β2M peptide covalently linked via a linker, and wherein the fusion protein (1) comprises less than 500 amino acids, or (2) lacks an HLA-E heavy chain.

Provided herein are also methods of genetically engineering a cell for an allogeneic transplant, comprising transducing the cell with a nucleic acid encoding a fusion protein, wherein the fusion protein comprises a presenting peptide and a β2M peptide covalently linked via a linker, and wherein the fusion protein (1) comprises less than 500 amino acids, or (2) lacks an HLA-E heavy chain.

Provided herein are also methods of genetically engineering a cell for an allogeneic transplant, comprising transducing the cell with the nucleic acids provided herein.

Provided herein are also methods of genetically engineering a cell for an allogeneic transplant, comprising transducing the cell with the vectors provided herein.

In some embodiments, methods provided herein further include inactivating at least one gene encoding a Class I MHC molecule or an MHC-like molecule in the cell. In some embodiments, the inactivated gene encodes a Class I MHC molecule selected from the group consisting of heavy chains of HLA-A, HLA-B, HLA-C, HLA-E, HLA-F, and HLA-G. In some embodiments, the inactivated gene encodes β2M. In some embodiments, the inactivated gene encodes an MHC-like molecule selected from the group consisting of a CD1 heavy chain, an MR1 heavy chain, an FcRn heavy chain and UL18.

In some embodiments, the gene is inactivated by DNA cleavage, DNA cleavage and repair, base editing, prime editing, RNA interference, or RNA editing. In some embodiments, the gene is inactivated by DNA cleavage using a rare endonuclease selected from the group consisting of a RNA-directed endonuclease, a TAL nuclease, a homing endonuclease, a zinc-finger nuclease, and a Mega-TAL nuclease. In some embodiments, the gene is inactivated by DNA cleavage and repair. In some embodiments, the gene is inactivated by using a CRISPR-Cas system. In some embodiments, the CRISPR-Cas system is a CRISPR-Cas9 system.

In some embodiments of methods provided herein, the cell is an immune cell. In some embodiments, the immune is selected from the group consisting of a T cell, a NK cell, a NKT cell, a B cell, a plasma cell, a monocyte, a macrophage, a dendritic cell and a granulocyte.

Provided herein are also methods of treating a subject in need of an allogeneic transplant, comprising preparing a genetically engineered cell according to methods of production disclosed herein, and administering the engineered cell to the subject.

### 5. BRIEF DESCRIPTION OF DRAWINGS

FIG. 1 is a schematic diagram of a fusion protein and a schematic diagram of an expression vector. FIG. 1, panel A is a schematic diagram of an exemplary fusion protein (or “chimeric molecule”; or “CM”) provided herein. FIG. 1, panel B is a schematic diagram of a vector having nucleic acid fragments encoding both the fusion protein (CM) and CAR.

FIG. 2 shows the co-expression of the fusion protein (CM) and the CAR in genetically engineered Jurkat T cells. Panel A shows results for untransduced cells in a control group. Panel B shows results for cells transduced with a CAR19-P2A-CM virus, wherein the CM comprised an HLA-E-restricted presenting peptide. As indicated by the arrow, the expression of HLA-E at the cell surface was detected with an HLA-E antibody. Panel C shows cells transduced with CAR19-P2A-CM-c, wherein the CM comprised an HLA-C-restricted presenting peptide. The expression of HLA-E at the cell surface was not detected by the HLA-E antibody.

FIG. 3 shows the expression of the fusion protein (CM) in primary T cells. Panel A shows total UCAR-T-CM cells. Panel B shows that about 80% of UCAR-T-CM cells having both the TCR gene and B2M gene knockout were double negative when stained with a CD3 antibody and an HLA-ABC antibody. Panel C shows the expression of the fusion protein (CM) in CD3 and HLA-ABC double negative UCAR-T-CM cells, as indicated by the positive staining for HLA-E.

FIG. 4 shows that UCAR-T cells expressing the fusion protein (CM) were resistant against killing by NK cells.

FIG. 5 shows that the UCAR-T cell expressing the fusion protein (CM) were resistant to growth inhibition by allogeneic PBMC cells and expanded rapidly upon stimulation by tumor cells.

FIG. 6 shows that the UCAR-T cell expressing the fusion protein (CM) efficiently arrested growth of tumor cells and also killed tumor cells in allogeneic PBMC environment.

FIG. 7 shows results from flow cytometry analysis, which demonstrated that the fusion protein (CM) and the HLA-E heavy chain molecule formed a complex on the cell membrane in β2M-knockout cells.

FIGS. 8A-8B show results from flow cytometry analysis, which demonstrated that different presenting peptides were associated with different levels of expression of the HLA-E complex on cell membrane. FIG. 8A shows the data from the flow cytometry analysis and FIG. 8B quantifies the data shown in FIG. 8A.

FIG. 9 shows results from flow cytometry analysis of expression of different fusion proteins in genetically engineered primary human T cells. Different fusion proteins (CM, CM12, CM13, CM14) contained different HLA-E-restricted presenting peptides. Cells in all groups had both TRAC and β2M knocked out using CRISPR/Cas9 and expressed a CD19-targeting CAR. The control UCAR-T group did not express any fusion protein.

FIG. 10 shows that UCAR-T cells expressing different fusion proteins (CM, CM12, CM13, CM14) were resistant to growth inhibition by allogeneic PBMC cells and expanded rapidly upon stimulation by tumor cells.

FIG. 11 shows results from flow cytometry analysis of co-expression of exogenous NY-ESO-1 targeted TCR (1G4) and the fusion protein (CM) in genetically engineered Jurkat T cells. NTC: non-transduced control (no lentiviral transduction). Panels A-D: wild-type Jurkat T cells; panels E-P: Jurkat T cells with TRAC and β2M double knock outs, hence CD3 and β2M double negative.

FIG. 12 shows that TRAC and β2M double-knocked-out primary human T cells expressing NY-ESO-1 targeted TCR (1G4) and the fusion protein CM were resistant to growth inhibition by allogeneic PBMC cells and expanded persistently upon stimulation by target cells.

FIG. 13 shows results from flow cytometry analysis of surface expression of HLA-ABC and CD3 in various groups of T cells with or without TRAC and β2M double knock-out.

FIG. 14 shows results from flow cytometry analysis of the expression of BCMA CAR and fusion protein CM as indicated by HLA-E expression in various groups of T cells with or without TRAC and β2M double knock-out. Notably, BCMA CAR and CM were both positive/co-expressed only in BCMA-CM CAR-T and BCMA-CM UCAR-T groups.

FIG. 15 shows the cytokine release profile of various groups of T cells. Notably, BCMA-CM CAR-T and BCMA-CM UCAR-T both released high amounts of IL-2 upon stimulation by BCMA-positive tumor cells.

FIG. 16 shows that BCMA-CM UCAR-T cells, which expressed CM, were resistant to NK cells as compared with UT cells which had no CM expression.

### 6. DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

The present disclosures relate to compositions and methods for improving success rate in allogeneic organ transplantation or allogeneic cell transplantation (e.g. universal CAR-T therapy). One advantage provided by the compositions and methods of the present disclosures is to overcome allograft rejection by the recipient's immune system. In particular, it is discovered that a fusion protein comprising a β2M polypeptide and a presenting peptide can be expressed in a cell for transplantation, which can form an MHC complex with an endogenously expressed MHC heavy chain on the cell surface, such that the MHC complex can bind an inhibitory receptor of the immune cells (e.g. NK cells and T cells) of the recipient, and prevent the recipient's immune cells from eliciting an immune response against the allogeneic cell.

For example, it is known that if an allograft cell lacks the expression of HLA-E on the cell surface, which is a nonclassical Class I MHC complex that binds the inhibitory receptor NKG2A on immune cells (e.g. NK cells or T cells), the allograft cell would be recognized and killed by the immune cells of the recipient (Saunders et al., Immunological Reviews (2015) 267: 148-166). In some embodiments, provided herein are genetically engineered cells that express fusion proteins having a β2M polypeptide and an HLA-E restricted presenting peptide. The fusion protein can form an HLA-E complex with an endogenously expressed HLA-E heavy chain on the cell surface, which binds to the inhibitory receptor NKG2A on a NK cell or T cell to inhibit the activation of the NK cell or T cell. In comparison to expressing a full MHC complex (e.g. β2M plus HLA-E heavy chain, at least 500 amino acids), the compositions and methods disclosed herein are advantageous at least because fusions proteins provided herein can be significantly smaller (e.g. less than 300 amino acids), which enables genetic engineering with greater accuracy and efficiency.

### 6.1 Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The terms “polypeptide,” “peptide,” “protein,” and their grammatical equivalents as used interchangeably herein refer to polymers of amino acids of any length, which can be linear or branched. It can include unnatural or modified amino acids, or be interrupted by non-amino acids. A polypeptide, peptide, or protein, can also be modified with, for example, disulfide bond formation, glycosylation, lipidation, acetylation, phosphorylation, or any other manipulation or modification.

The term “fusion protein” and its grammatical equivalents as used herein refer to a protein, peptide or polypeptide that has an amino acid sequence derived from two or more separate proteins, peptides or polypeptides. The fusion protein also include linking regions of amino acids between amino acid portions derived from separate proteins, peptides or polypeptides. Such linking regions of amino acids are referred herein as “linkers.”

The term “Major Histocompatibility Complex,” “MHC complex,” and “MHC,” are used interchangeably herein consistently with their usage commonly accepted in the art. Briefly, the terms refer to a group of closely linked genes that code for cell surface proteins essential for the acquired immune system to recognize foreign molecules in vertebrates, which in turn determines histocompatibility. The main function of MHC is antigen presentation, and extremely rich polymorphism has been observed. The human MHC is also referred to as a human leukocyte antigen (“HLA”). MHC complexes are heterodimers and composed of two “MHC proteins.” It is understood that an “MHC gene” refers to a polynucleotide encoding an MHC protein.

MHC complexes include Class I MHC (or MHC-I Class), Class II MHC (or MHC-II Class) and MHC-like complexes (or “MHC analogs,” or “MHC homologs”). Class I MHC complexes are heterodimeric glycoproteins consisting of two peptide chains associated by non-covalent bonds, a polymorphic heavy chain (i.e. “Class I MHC heavy chain,” also referred to as “Class I MHC alpha chain”), and a light chain, which is beta-2-microglobulin (β2M). Class I MHC complexes display peptide fragments of proteins from within the cell to immune cells, which triggers an immediate response from the immune system against a particular non-self antigen displayed by Class I MHC complexes. In human, classic Class I MHC (also known as MHC-Ia) include HLA-A, HLA-B and HLA-C; and nonclassical Class I MHC (also known as MHC-Ib) include HLA-E, HLA-F and HLA-G.

MHC-like complexes, also called MHC analogs, or MHC homologs, are structurally similar to the Class I MHC complexes. Some MHC-like complexes are also consisted of a heavy chain (i.e. “MHC-like heavy chain”) non-covalently associated with the β2M protein. Exemplary MHC-like complexes include, but are not limited to, CD1, MR1, Neonatal Fc-receptor (FcRn), and human cytomegalovirus (HCMV)-derived UL18. CD1 is structurally similar to other Class I MHC complexes and consists of a heavy chain and β2M. CD1 presents lipid antigens and further includes CD1a, CD1b, CD1c, CD1d and CD1e. MR1 (MHC-related 1) is structurally similar to the Class I MHC complexes and consists of a heavy chain and β2M. MR1 mainly presents small aromatic molecules, which activate mucosal associated invariant T cells (MAIT). These small aromatic molecules are in general smaller than the polypeptide presented by Class I MHC or the lipid presented by CD1. Neonatal Fc-receptor (FcRn) transports immunoglobulin G (IgG) across cell layers, extending IgG half-life in circulation and providing newborns with humoral immunity. Like other Class I MHC, FcRN consists of a heavy chain and β2M. UL18 shares homology with MHC heavy chains, and binds to β2M to form a complex expressed on the cell surface, which allows the cell to evade NK cell-mediated cytolysis.

The term “beta-2-microglobulin (β2M) peptide” refers to the full length protein of beta-2-microglobulin, or a functional fragment or variant thereof β2M is a light chain of the Class I MHC complexes. Inactivation of the B2M gene effectively eliminates the expression of Class I MHC from the cell surface. Cells with no expression of Class I MHC on the surface are recognized and killed by NK cells. Human β2M has a molecular weight of 11,800 Daltons and consists of 119 amino acids (SEQ ID NO:7). The first 20 amino acids at the N-terminus form the signal peptide of human β2M, which can be cleaved. The mature form of human β2M does not include the signal peptide and consists of 99 amino acids (SEQ ID NO:81).

Exemplary functional fragments of β2M include, for example, the truncated form ΔN602M (93 amino acids), which lack the 6 N-terminal amino acids of the mature β2M, and and ΔN1002M (89 amino acids), which lack the 10 N-terminal amino acids of the mature β2M (Sulatskaya et al., Int. J. Mol. Sci. (2018) 19: 2762). Exemplary variants includes, for example, the following isoforms provided on UniProt: entries F5H6I0 (101 amino acids), HOYLF3 (71 amino acids), B4E0X1 (122 amino acids), A6XMH4 (124 amino acid), A6XMH5(92 amino acids), A6XND9(101 amino acids), Q9UM88 (29 amino acids), Q16446 (51 amino acids), and J3KNU0 (57 amino acids).

The term “variant” as used herein in relation to a protein or a polypeptide with particular sequence features (the “reference protein” or “reference polypeptide”) refers to a different protein or polypeptide comprising one or more (such as, for example, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. A functional fragment or a functional variant of a protein or polypeptide maintains the basic structural and functional properties of the reference protein or polypeptide. By way of example, a variant of human β2M can have one or more (such as, for example, about 1 to about 25, about 1 to about 20, about 1 to about 15, about 1 to about 10, or about 1 to about 5) changes to the amino acid sequence of wild type human β2M. The variant of human β2M has the basic structural and functional properties of human β2M. For example, like full length human β2M, a human β2M variant can also bind to an MHC heavy chain to form an MHC complex that can be recognized by immune cells.

The term “presenting peptide” as used herein refers to short peptides that can stably bind to the antigen binding groove of an MHC to form a stable MHC complex that can be recognized by receptors on immune cells. The presenting peptides generally have 7-30 amino acids. In some embodiments, the presenting peptides have 7-20 amino acids, 7-17 amino acids, 7-15 amino acids, 7-12 amino acids, or 8-10 amino acids.

Each MHC has its own matched presenting peptides. A presenting peptide that is “restricted” to a particular MHC molecule means that the presenting peptide can form a stable complex with this particular MHC molecule. For example, an HLA-C restricted presenting peptide can form a stable complex with HLA-C on the cell surface, and an HLA-E restricted presenting peptide can form a stable complex with HLA-E on the cell surface. Presenting peptides that are restricted to a particular HLA can be identified by selecting the presenting peptides that stably bind to the HLA. A presenting peptide that is restricted to a particular Class I MHC or MHC-like complex can be covalently linked to β2M to form a fusion protein, which can form a stable complex with the particular MHC heavy chain. For example, a fusion protein having an HLA-C restricted presenting peptide covalently linked to β2M can form a stable complex with an HLA-C heavy chain on the cell surface, and a fusion protein having an HLA-E restricted presenting peptide covalently linked to β2M can form a stable complex with an HLA-E heavy chain on the cell surface.

The term “inhibitory receptor,” as used herein in connection with an immune cell refers to the receptor molecule expressed on the surface of the immune cell that can inhibit the activity of the immune cells upon binding to its ligand. The immune system employs a variety of inhibitory mechanisms to control immune responses as well as to ensure immune tolerance and homeostasis, including inhibitory pathways mediated via the inhibitory receptors. For example, NK cells are regulated by the autologous Class I MHC-binding receptors, which confer dominant suppression of NK cells upon binding to self MHC class I molecules. Class I MHC-binding inhibitory receptors are expressed by not only NK cells, but also subsets of T cells, especially CD8+ T cells. There are at least two families of these inhibitory receptors, including the killer immunoglobulin-like receptor (KIR) family and the C type lectin-like family (e.g. NKG2A/B, CD94).

The term “specifically bind,” and its grammatical equivalents as used herein, mean that a polypeptide or molecule interacts more frequently, more rapidly, with greater duration, with greater affinity, or with some combination of the above to the epitope, protein, or target molecule than with alternative substances, including related and unrelated proteins. A binding moiety (e.g. antibody) that specifically binds a target molecule (e.g. antigen) can be identified, for example, by immunoassays, ELISAs, SPR (e.g., Biacore), or other techniques known to those of skill in the art. Typically, a specific reaction will be at least twice background signal or noise and can be more than 10 times background. A binding moiety that specifically binds a target molecule can bind the target molecule at a higher affinity than its affinity for a different molecule. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of at about 30 μM or less. In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less.

The terms “polynucleotide,” “nucleic acid,” and their grammatical equivalents as used interchangeably herein mean polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical,” percent “identity,” and their grammatical equivalents as used herein in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides provided herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

The term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. Unless expressly indicated otherwise, the term “antibody” as used herein include “antigen-binding domain” of intact antibodies. The term “antigen-binding domain” as used herein refers to a portion or fragment of an intact antibody that is the antigenic determining variable region of an intact antibody. Examples of antigen-binding domains include, but are not limited to, Fab, Fab′, F(ab′)₂, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH).

The term “vector,” and its grammatical equivalents as used herein refer to a vehicle that is used to carry genetic material (e.g. a nucleic acid sequences), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acids into a host cell can be confirmed using methods well known in the art. It is understood by those skilled in the art that the nucleic acids are expressed in a sufficient amount to produce a desired product (e.g. a fusion protein as described herein), and it is further understood that expression levels can be optimized to obtain sufficient expression using methods well known in the art.

The term “operably linked,” and its grammatical equivalents refer to functional linkage between a regulatory sequence (such as a promoter, and/or enhancer) and a second nucleic acid sequence, wherein the regulatory sequence directs transcription of the second nucleic acid sequence; or the linkage between two nucleic acid sequences to be co-expressed that are in the same reading frame. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “exogenous” and its grammatical equivalents as used herein are intended to mean that the referenced molecule is introduced into the host cell. The molecule can be introduced, for example, by introduction of an encoding nucleic acid into the host genetic material such as by integration into a host chromosome or as non-chromosomal genetic material such as a plasmid. When used in reference to expression of an encoding nucleic acid, the term refers to introduction of the encoding nucleic acid in an expressible form into the cell.

The term “endogenous” and its grammatical equivalents as used herein refer to a referenced molecule that is naturally present in the host cell. Similarly, the term when used in reference to expression of an encoding nucleic acid refers to expression of an encoding nucleic acid naturally contained within the cell.

The term “genetic engineering” or its grammatical equivalents when used in reference to a cell is intended to mean alteration of the genetic materials of the cell that is not normally found in a naturally occurring cell. Genetic alterations include, for example, modifications introducing expressible nucleic acids, other nucleic acid additions, nucleic acid mutations/alterations, nucleic acid deletions and/or other functional disruption of the cell's genes. Such modifications can be done in, for example, coding regions and functional fragments thereof of a gene. Additional modifications can be done in, for example, non-coding regulatory regions in which the modifications alter expression of a gene.

The terms “transduce,” “transfect” and their grammatical equivalents as used herein refer to a process by which exogenous nucleic acid is transferred or introduced into the host cell. A “transduced” or “transfected” cell is one which has been transduced with an exogenous nucleic acid. The cell can be the primary subject cell, or its progeny.

As used herein, the terms “inactivate,” “disrupt,” and their grammatical equivalents when used in connection with a gene, are intended to mean a genetic alteration that renders the encoded gene product inactive or attenuated. The genetic alteration can be, for example, deletion of the entire gene, deletion of a regulatory sequence required for transcription or translation, deletion of a portion of the gene which results in a truncated gene product, or by any of various mutation strategies that inactivate or attenuate the encoded gene product. A gene disruption also includes a null mutation, which refers to a mutation within a gene or a region containing a gene that results in the gene not being transcribed into RNA and/or translated into a functional gene product. Such a null mutation can arise from many types of mutations including, for example, inactivating point mutations, deletion of a portion of a gene, entire gene deletions, or deletion of chromosomal segments.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated. For example, when used in reference to a cancer or tumor, the term “treat” and its grammatical equivalents refer to an action that reduces the severity of the cancer or tumor, or retards or slows the progression of the cancer or tumor, including (a) inhibiting the growth, or arresting development of the cancer or tumor, (b) causing regression of the cancer or tumor, or (c) delaying, ameliorating or minimizing one or more symptoms associated with the presence of the cancer or tumor.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (TP); transdermal dosage forms, including creams, jellies, powders, or patches; buccal dosage forms; inhalation powders, sprays, suspensions, and rectal suppositories.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “pharmaceutically acceptable excipient” refers to a material that is suitable for drug administration to an individual along with an active agent without causing any undesirable biological effects or interacting in a deleterious manner with any of the other components of the pharmaceutical composition.

The term “subject” as used herein refers to any animal (e.g., a mammal), including, but not limited to, humans, non-human primates, canines, felines, rodents, and the like, which is to be the recipient of a particular treatment. A subject can be a human. A subject can be a patient with a particular disease. A subject can also be a recipient of a transplant.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOS. It is understood that one skilled in the art can readily identify homologous sequences by reference to sequence sources, including but not limited to GenBank (ncbi.nlm.nih.gov/genbank/) and EMBL (embl.org/).

### 6.2 Fusion Proteins

It is found that, when expressed in a host cell, fusion proteins comprising a presenting peptide covalently linked to a β2M peptide via a linker, can form a complex with an endogenous MHC heavy chain on the cell surface, wherein said complex can bind an inhibitory receptor of an immune cell to inhibit the immune cell. For example, a fusion protein having human β2M covalently linked to an HLA-E restricted presenting peptide can be expressed in a T cell, which can form an HLA-E complex with an endogenous HLA-E heavy chain on the T cell surface. When the genetically engineered T cell is administered to a recipient as an allogeneic transplant, the HLA-E complex on the T cell surface can then be recognized by the immune cells (e.g NK cells) of the recipient, inhibiting the immune cells and protecting the T cell from a potential allogeneic immune response from these immune cells (e.g. NK cell-mediated cell lysis).

As such, provided herein are fusion proteins comprising a presenting peptide covalently linked to a β2M peptide via a linker, wherein the fusion proteins bind an MHC heavy chain to form an MHC complex that binds an inhibitory receptor of an immune cell to inhibit the immune cell. In some embodiments, the fusion protein has less than 500 amino acids. In some embodiments, the fusion protein has less than 400 amino acids. In some embodiments, the fusion protein has less than 300 amino acids. In some embodiments, the fusion protein has less than 200 amino acids. In some embodiments, the fusion protein has about 100 to about 300 amino acids. In some embodiments, the fusion protein has about 100 to about 200 amino acids. In some embodiments, the fusion protein has about 120 to about 180 amino acids. In some embodiments, the fusion protein has about 120 to about 160 amino acids. In some embodiments, the fusion protein has about 140 to about 160 amino acids. In some embodiments, the fusion protein lacks an MHC heavy chain. In some embodiments, the fusion protein lacks a Class I MHC heavy chain. In some embodiments, the fusion protein lacks an MHC-like heavy chain. In some embodiments, the fusion protein lacks an HLA-A heavy chain. In some embodiments, the fusion protein lacks an HLA-B heavy chain. In some embodiments, the fusion protein lacks an HLA-C heavy chain. In some embodiments, the fusion protein lacks an HLA-E heavy chain. In some embodiments, the fusion protein lacks an HLA-F heavy chain. In some embodiments, the fusion protein lacks an HLA-G heavy chain. In some embodiments, the fusion protein lacks a CD1 heavy chain. In some embodiments, the fusion protein lacks an MR1 heavy chain. In some embodiments, the fusion protein lacks an FcRn heavy chain. In some embodiments, the fusion protein lacks UL18.

Provided herein are fusion proteins comprising a presenting peptide covalently linked to a β2M peptide via a linker, wherein the fusion proteins bind an MHC heavy chain to form an MHC complex that binds an inhibitory receptor of an immune cell to inhibit the immune cell. In some embodiments, the immune cell is a T cell; and provided herein are fusion proteins that bind an MHC heavy chain to form an MHC complex, which binds an inhibitory receptor of a T cell to inhibit the T cell. In some embodiments, the immune cell is an NK cell; and provided herein are fusion proteins that bind an MHC heavy chain to form an MHC complex, which binds an inhibitory receptor of an NK cell to inhibit the NK cell.

In some embodiments, the inhibitory receptor of the immune cell is NKG2A. Provided herein are fusion proteins comprising a presenting peptide covalently linked to a β2M peptide via a linker, wherein the fusion proteins bind an MHC heavy chain to form an MHC complex that binds the NKG2A receptor of an immune cell to inhibit the immune cell. In some embodiments, the immune cell is a T cell. In some embodiments, the immune cell is an NK cell. A human NKG2A can have an amino acid sequence corresponding to the sequence having GenBank No. AAL65234.1 (Accession: AAL65234.1 GI: 18182682; see below).

```
(SEQ ID NO: 82)
    1 MDNQGVIYSD LNLPPNPKRQ QRKPKGNKSS
        ILATEQEITY AELNLQKASQ DFQGNDKTYH
  61 CKDLPSAPEK LIVGILGIIC LILMASVVTI
        VVIPSTLIQR HNNSSLNTRT QKARHCGHCP
121 EEWITYSNSC YYIGKERRTW EESLLACTSK
        NSSLLSIDNE EEMKELSIIS PSSWIGVERN
181 SSHHPWVTMN GLAFKHEIKD SDNAELNCAV
        LQVNRLKSAQ CGSSIIYHCK HKL
```

In some embodiments, the inhibitory receptor of the immune cell is a KIR receptor. The inhibitory KIR receptor can be selected from the group consisting of KIR2DL1, KIR2DL2, KIR2DL3, KIR2DL4, KIR2DL5, KIR3DL1, KIR3DL2, and KIR3DL3.

In some embodiments, the inhibitory receptor of the immune cell is KIR2DL1. Provided herein are fusion proteins comprising a presenting peptide covalently linked to a β2M peptide via a linker, wherein the fusion proteins bind an MHC heavy chain to form an MHC complex that binds the KIR2DL1 receptor of an immune cell to inhibit the immune cell. In some embodiments, the immune cell is a T cell. In some embodiments, the immune cell is an NK cell. A human KIR2DL1 can have an amino acid sequence corresponding to the sequence having GenBank No. SPC71652.1 (Accession: SPC71652.1 GI: 1373834531; see below).

```
(SEQ ID NO: 83)
    1 MSLLVVSMAC VGFFLLQGAW PHEGVHRKPS
        LLAHPGRLVK SEETVILQCW SDVMFEHELL
  61 HREGMENDTL RLIGEHHDGV SKANFSISRM
        TQDLAGTYRC YGSVTHSPYQ VSAPSDPLDI
121 VIIGLYEKPS LSAQLGPTVL AGENVTLSCS
        SRSSYDMYHL SREGEAHERR LPAGPKVNGT
181 FQADFPLGPA THGGTYRCFG SFHDSPYEWS
        KSSDPLLVSV TGNPSNSWPS PTEPSSKTGN
241 PRHLHILIGT SVVIILFILL FFLLHHWCSN
        KKNAAVMDQE SAGNRTANSE DSDEQDPQEV
301 TYTQLNHCVF TQRKITRPSQ RPKTPPTDII
        VYTELPNAES RSKVVSCP
```

In some embodiments, the inhibitory receptor of the immune cell is KIR2DL2. Provided herein are fusion proteins comprising a presenting peptide covalently linked to a β2M peptide via a linker, wherein the fusion proteins bind an MHC heavy chain to form an MHC complex that binds the KIR2DL2 receptor of an immune cell to inhibit the immune cell. In some embodiments, the immune cell is a T cell. In some embodiments, the immune cell is an NK cell. A human KIR2DL2 can have an amino acid sequence corresponding to the sequence having GenBank No. ACI49717.1 (Accession: ACI49717.1 GI: 209512829; see below).

```
(SEQ ID NO: 84)
    1 MSLMVVSMAC VGFFLLQGAW PHEGVHRKPS
        LLAHPGRLVK SEETVILQCW SDVRFEHELL
  61 HREGKFKDTL HLIGEHHDGV SKANFSIGPM
        MQDLAGTYRC YGSVTHSPYQ LSAPSDPLDI
121 VITGLYEKPS LSAQPGPTVL AGESVTLSCS
        SRSSYDMYHL SREGEAHECR FSAGPKVNGT
181 FQADFPLGPA THGGTYRCFG SFRDSPYEWS
        NSSDPLLVSV IGNPSNSWPS PTEPSSKTGN
241 PRHLHILIGT SVVIILFILL FFLLHRWCSN
        KKNAAVMDQE SAGNRTANSE DSDEQDPQEV
301 TYTQLNHCVF TQRKITRPSQ RPKTPPTDII
        VYTELPNAES RSKVVSCP
```

In some embodiments, the inhibitory receptor of the immune cell is KIR2DL3. Provided herein are fusion proteins comprising a presenting peptide covalently linked to a β2M peptide via a linker, wherein the fusion proteins bind an MHC heavy chain to form an MHC complex that binds the KIR2DL3 receptor of an immune cell to inhibit the immune cell. In some embodiments, the immune cell is a T cell. In some embodiments, the immune cell is an NK cell. A human KIR2DL3 can have an amino acid sequence corresponding to the sequence having GenBank No. ADN34702.1 (Accession: ADN34702.1 GI: 307141824; see below).

```
(SEQ ID NO: 85)
    1 MSLMVVSMVC VGFFLLQGAW PHEGVHRKPS
        LLAHPGPLVK SEETVILQCW SDVRFQHELL
  61 HREGKFKDTL HLIGEHHDGV SKANFSIGPM
        MQDLAGTYRC YGSVTHSPYQ LSAPSDPLDI
121 VITGLYEKPS LSAQPGPTVL AGESVTLSCS
        SRSSYDMYHL SREGEAHERR FSAGPKVNGT
181 FQADFPLGPA THGGTYRCFG SFRDSPYEWS
        NSSDPLLVSV TGNPSNSWPS PTEPSSETGN
241 PRHLHVLIGT SVVIILFILL LFFLLHRWCC
        NKKNAVVMDQ EPAGNRTVNR EDSDEQDPQE
301 VTYAQLNHCV FTQRKITRPS QRPKTPPTDI
        IVYTELPNAE P
```

In some embodiments, the inhibitory receptor of the immune cell is KIR2DL4. Provided herein are fusion proteins comprising a presenting peptide covalently linked to a β2M peptide via a linker, wherein the fusion proteins bind an MHC heavy chain to form an MHC complex that binds the KIR2DL4 receptor of an immune cell to inhibit the immune cell. In some embodiments, the immune cell is a T cell. In some embodiments, the immune cell is an NK cell. A human KIR2DL4 can have an amino acid sequence corresponding to the sequence having GenBank No. ABW73959.1 (Accession: ABW73959.1 GI: 158551989; see below).

```
(SEQ ID NO: 86)
    1 MSMSPTVIIL ACLGFFLDQS VWAHVGGQDK
        PFCSAWPSAV VPQGGHATLR CHCRRGENIF
  61 TLYKKDGVPV PELYNRIFWN SFLISPVTPA
        HAGTYRCRGF HPHSPTEWSA PSNPLVIMVT
121 GLYEKPSLTA RPGPTVRAGE NVTLSCSSQS
        SFDIYHLSRE GEAHELRLPA VPSINGTFQA
181 DFPLGPATHG ETYRCFGSFH GSPYEWSDPS
        DPLPVSVTGN PSSSWPSPTE PSFKTGIARH
241 LHAVIRYSVA IILFTILPFF LLHRWCSKKK
        NAAVMNQEPA GHRTVNREDS DEQDPQEVTY
301 AQLDHCIFTQ RKITGPSQRS KRPSTDTSVC
        IELPNAEPRA LSPAHEHHSQ ALMGSSRETT
361 ALSQTQLASS NVPAAGI
```

In some embodiments, the inhibitory receptor of the immune cell is KIR2DL5. Provided herein are fusion proteins comprising a presenting peptide covalently linked to a β2M peptide via a linker, wherein the fusion proteins bind an MHC heavy chain to form an MHC complex that binds the KIR2DL5 receptor of an immune cell to inhibit the immune cell. In some embodiments, the immune cell is a T cell. In some embodiments, the immune cell is an NK cell. A human KIR2DL5 can have an amino acid sequence corresponding to the sequence having GenBank No. SH029775.1 (Accession: SH029775.1 GI: 1313716695; see below).

```
(SEQ ID NO: 87)
    1 MSLMVVSMAC VGFFLLQGAW THEGGQDKPL
        LSAWPSAVVP RGGHVTLLCR SRLGFTIFSL
  61 YKEDGVPVPE LYNKIFWKSI LMGPVTPAHA
        GTYRCRGSHP RSPIEWSAPS NPLVIVVTGL
121 FGKPSLSAQP GPTVRTGENV TLSCSSRSSF
        DMYHLSREGR AHEPRLPAVP SVNGTFQADE
181 PLGPATHGGT YTCFGSLHDS PYEWSDPSDP
        LLVSVTGNSS SSSSSPTEPS SKTGIRRHLH
241 ILIGTSVAII LFIILFFFLL HCCCSNKKNA
        AVMDQEPAGD RTVNREDSDD QDPQEVTYAQ
301 LDHCVFTQTK ITSPSQRPKT PPTDTTMYME
        LPNAKPRSLS PAHKHHSQAL RGSSRETTAL
361 SQNRVASSHV PAAGI
```

In some embodiments, the inhibitory receptor of the immune cell is KIR3DL1. Provided herein are fusion proteins comprising a presenting peptide covalently linked to a β2M peptide via a linker, wherein the fusion proteins bind an MHC heavy chain to form an MHC complex that binds the KIR3DL1 receptor of an immune cell to inhibit the immune cell. In some embodiments, the immune cell is a T cell. In some embodiments, the immune cell is an NK cell. A human KIR3DL1 can have an amino acid sequence corresponding to the sequence having GenBnk No. ADM64608.1 (Accession: ADM64608.1 GI: 305690575 see below).

```
(SEQ ID NO: 88)
    1 MLLMVVSMAC VGFFLVQRAG PHVGGQDKPF
        LSAWPSAVVP RGGHVTLRCH YRHRENNFML
  61 YKEDRIHVPI FHGRLFQESF NMSPVTTAHA
        GNYTCRGSHP HSPTGWSAPS NPVVIMVTGN
121 HRKPSLLAHP GPLVKSGERV ILQCWSDIMF
        EHFFLHKEGI SKDPSRLVGQ IHDGVSKANF
181 SIGPMMLALA GTYRCYGSVT HTPYQLSAPS
        DPLDIVVTGP YEKPSLSAQP GPKVQAGESV
241 TLSCSSRSSY DMYHLSREGG AHERRLPAVR
        KVNRTFQADF PLGPATHGGT YRCFGSFRHS
301 PYEWSDPSDP LLVSVTGNPS SSWPSPTEPS
        SKSGNPRHLH VLIGTSVVII LFILLLFFLL
361 HLWCSNKKNA AVMDQEPAGN RTANSEDSDE
        QDPQEVTYTQ LNHCVFTQRK ITRPSQRPKT
421 PPTDTILYTE LPNAKPRSKV VSCP
```

In some embodiments, the inhibitory receptor of the immune cell is KIR3DL2. Provided herein are fusion proteins comprising a presenting peptide covalently linked to a β2M peptide via a linker, wherein the fusion proteins bind an MHC heavy chain to form an MHC complex that binds the KIR3DL2 receptor of an immune cell to inhibit the immune cell. In some embodiments, the immune cell is a T cell. In some embodiments, the immune cell is an NK cell. A human KIR3DL2 can have an amino acid sequence corresponding to the sequence having GenBank No. CUX91181.1 (Accession: CUX91181.1 GI: 998428963; see below).

```
(SEQ ID NO: 89)
    1 MSLTVVSMAC VGFFLLQGAW PLMGGQDKPF
        LSARPSTVVP RGGHVALQCH YRRGENNEML
  61 YKEDRSHVPI FHGRIFQESF IMGPVTPAHA
        GTYRCRGSRP HSLTGWSAPS NPLVIMVTGN
121 HRKPSLLAHP GPLLKSGETV ILQCWSDVMF
        EHFFLHREGI SEDPSRLVGQ IHDGVSKANF
181 SIGPLMPVLA GTYRCYGSVP HSPYQLSAPS
        DPLDIVITGL YEKPSLSAQP GPTVQAGENV
241 TLSCSSWSSY DIYHLSREGE AHERRLRAVP
        KVNRTFQADF PLGPATHGGT YRCFGSFHAL
301 PCVWSNSSDP LLVSVTGNPS SSWPSPTEPS
        SKSGICRHLH VLIGTSVVIF LFILLLFFLL
361 YRWCSNKKNA AVMDQEPAGD RTVNRQDSDE
        QDPQEVTYAQ LDHCVFIQRK ISRPSQRPKT
421 PLTDTSVYTE LPNAEPRSKV VSCPRAPQSG
        LEGVF
```

In some embodiments, the inhibitory receptor of the immune cell is KIR3DL3. Provided herein are fusion proteins comprising a presenting peptide covalently linked to a β2M peptide via a linker, wherein the fusion proteins bind an MHC heavy chain to form an MHC complex that binds the KIR3DL3 receptor of an immune cell to inhibit the immune cell. In some embodiments, the immune cell is a T cell. In some embodiments, the immune cell is an NK cell. A human KIR3DL3 can have an amino acid sequence corresponding to the sequence having GenBank No. SH029793.1 (Accession: SH029793.1 GI: 1313716772; see below).

```
(SEQ ID NO: 90)
    1 MSLMVVSMAC VGFFLLEGPW PHVGGQDKPF
        LSAWPSTVVS EGQHVTLQCR SRLGENEFSL
  61 SKEDGMPVPE LYNRIFRNSF LMGPVTPAHA
        GTYRCCSSHP HSPTGWSAPS NPVVIMVTGV
121 HRKPSLLAHP GPLVKSGETV ILQCWSDVRF
        ERFLLHREGI TEDPLRLVGQ LHDAGSQVNY
181 SMGPMTPALA GTYRCFGSVT HLPYELSAPS
        DPLDIVVVGL YGKPSLSAQP GPTVQAGENV
241 TLSCSSRSLF DIYHLSREAE AGELRLTAVL
        RVNGTFQANF PLGPVTHGGN YRCFGSFRAL
301 PHAWSDPSDP LPVSVTGNSR YLHALIGTSV
        VIIPFAILLF FLLHRWCANK KNAVVMDQEP
361 AGNRTVNRED SDEQDPQEVT YAQLNHCVFT
        QRKITRPSQR PKTPPTDTSV
```

In some embodiments, the inhibitory receptor of the immune cell is leukocyte immunoglobulin-like receptor 1 “LIR1.” Provided herein are fusion proteins comprising a presenting peptide covalently linked to a β2M peptide via a linker, wherein the fusion proteins bind an MHC heavy chain to form an MHC complex that binds the LIR1 receptor of an immune cell to inhibit the immune cell. In some embodiments, the immune cell is a T cell. In some embodiments, the immune cell is an NK cell. A human LIR1 can have an amino acid sequence corresponding to the sequence having GenBank No. AAG08984.1 (Accession: AAG08984.1 GI: 9954210; see below).

```
(SEQ ID NO: 91)
    1 MTPILTVLIC LGLSLGPRTH VQAGHLPKPT
        LWAEPGSVIT QGSPVTLRCQ GGQETQEYRL
  61 YREKKTALWI TRIPQELVKK GQFPIPSITW
        EHAGRYRCYY GSDTAGRSES SDPLELVVTG
121 AYIKPTLSAQ PSPVVNSGGN VILQCDSQVA
        FDGFSLCKEG EDEHPQCLNS QPHARGSSRA
181 IFSVGPVSPS RRWWYRCYAY DSNSPYEWSL
        PSDLLELLVL GVSKKPSLSV QPGPIVAPEE
241 TLTLQCGSDA GYNRFVLYKD GERDFLQLAG
        AQPQAGLSQA NFTLGPVSRS YGGQYRCYGA
301 HNLSSEWSAP SDPLDILIAG QFYDRVSLSV
        QPGPTVASGE NVTLLCQSQG WMQTELLTKE
361 GAADDPWRLR STYQSQKYQA EFPMGPVTSA
        HAGTYRCYGS QSSKPYLLTH PSDPLELVVS
421 GPSGGPSSPT TGPTSTSGPE DQPLTPTGSD
        PQSGLGRHLG VVIGILVAVI LLLLLLLLLF
481 LILRHRRQGK HWTSTQRKAD FQHPAGAVGP
        EPTDRGLQWR SSPAADAQEE NLYAAVKHTQ
541 PEDGVEMDTR SPHDEDPQAV TYAEVKHSRP
        RREMASPPSP LSGEFLDTKD RQAEEDRQMD
601 TEAAASEAPQ DVTYAQLHSL TLRREATEPP
        PSQEGPSPAV PSIYATLAIH
```

Provided herein are fusion proteins comprising a presenting peptide covalently linked to a β2M peptide via a linker, wherein the fusion proteins bind an MHC heavy chain to form an MHC complex that binds an inhibitory receptor of an immune cell to inhibit the immune cell. In some embodiments, the MHC heavy chain is a classical Class I MHC heavy chain, a non-classical Class I MHC heavy chain, or an MHC-like heavy chain. In some embodiments, provided herein are fusion proteins comprising a presenting peptide covalently linked to a β2M peptide via a linker, wherein the fusion proteins bind a classical Class I MHC heavy chain to form an MHC complex that binds an inhibitory receptor of an immune cell to inhibit the immune cell. The classical Class I MHC heavy chain can be selected from the group consisting of an HLA-A heavy chain, an HLA-B heavy chain, and an HLA-C heavy chain.

In some embodiments, the classical Class I MHC heavy chain is an HLA-A heavy chain. Provided herein are fusion proteins comprising an HLA-A-restricted presenting peptide covalently linked to a β2M peptide via a linker, wherein the fusion protein binds an HLA-A heavy chain to form an HLA-A complex that binds an inhibitory receptor of an immune cell to inhibit the immune cell. In some embodiments, the fusion protein does not have an HLA-A heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. Human HLA-A heavy chain is highly polymorphic. For example, the amino acid sequence of one allelic form of human HLA-A heavy chain corresponds to the sequence having GenBank No. BAB63400.1 (Accession: BAB63400.1 GI: 15277272).

In some embodiments, the classical Class I MHC heavy chain is an HLA-B heavy chain. Provided herein are fusion proteins comprising an HLA-B-restricted presenting peptide covalently linked to a β2M peptide via a linker, wherein the fusion proteins bind an HLA-B heavy chain to form an HLA-B complex that binds an inhibitory receptor of an immune cell to inhibit the immune cell. In some embodiments, the fusion protein does not have an HLA-B heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, about 120 to about 160 amino acids, or about 140 to about 160 amino acids. Human HLA-B heavy chain is highly polymorphic. For example, the amino acid sequence of one allelic form of human HLA-B heavy chain corresponds to the sequence having GenBank No. AAA59682.1 (Accession: AAA59682.1 GI: 403145).

In some embodiments, the classical Class I MHC heavy chain is an HLA-C heavy chain. Provided herein are fusion proteins comprising an HLA-C-restricted presenting peptide covalently linked to a β2M peptide via a linker, wherein the fusion proteins bind an HLA-C heavy chain to form an HLA-C complex that binds an inhibitory receptor of an immune cell to inhibit the immune cell. In some embodiments, the fusion protein does not have an HLA-C heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. Human HLA-C heavy chain is highly polymorphic. For example, the amino acid sequence of one allelic form of human HLA-C heavy chain corresponds to the sequence having GenBank No. BAB63310.1 (Accession: BAB63310.1 GI: 15277217).

In some embodiments, provided herein are fusion proteins comprising a presenting peptide covalently linked to a β2M peptide via a linker, wherein the fusion proteins bind a nonclassical Class I MHC heavy chain to form an MHC complex that binds an inhibitory receptor of an immune cell to inhibit the immune cell. The nonclassical Class I MHC heavy chain can be selected from the group consisting of an HLA-E heavy chain, an HLA-F heavy chain, and an HLA-G heavy chain.

In some embodiments, the nonclassical Class I MHC heavy chain is an HLA-E heavy chain. Provided herein are fusion proteins comprising an HLA-E-restricted presenting peptide covalently linked to a β2M peptide via a linker, wherein the fusion proteins bind an HLA-E heavy chain to form an HLA-E complex that binds an inhibitory receptor of an immune cell to inhibit the immune cell. In some embodiments, the fusion protein does not have an HLA-E heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, about 120 to about 160 amino acids, or about 140 to about 160 amino acids. Human HLA-E heavy chain is polymorphic. For example, the amino acid sequence of one allelic form of human HLA-E heavy chain corresponds to the sequence having GenBank No. ARB08449.1 (Accession: ARB08449.1 GI: 1168024009).

In some embodiments, the nonclassical Class I MHC heavy chain is an HLA-F heavy chain. Provided herein are fusion proteins comprising an HLA-F-restricted presenting peptide covalently linked to a β2M peptide via a linker, wherein the fusion proteins bind an HLA-F heavy chain to form an HLA-F complex that binds an inhibitory receptor of an immune cell to inhibit the immune cell. In some embodiments, the fusion protein does not have an HLA-F heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. Human HLA-F heavy chain is polymorphic. For example, the amino acid sequence of one allelic form of human HLA-F heavy chain corresponds to the sequence having GenBank No. BAB63337.1 (Accession: BAB63337.1 GI: 15277244)

In some embodiments, the nonclassical Class I MHC heavy chain is an HLA-G heavy chain. Provided herein are fusion proteins comprising an HLA-G-restricted presenting peptide covalently linked to a β2M peptide via a linker, wherein the fusion proteins bind an HLA-G heavy chain to form an HLA-G complex that binds an inhibitory receptor of an immune cell to inhibit the immune cell. In some embodiments, the fusion protein does not have an HLA-G heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. Human HLA-G heavy chain is highly polymorphic. For example, the amino acid sequence of one allelic form of human HLA-G heavy chain corresponds to the sequence having GenBank No. BAB63336.1 (Accession: BAB63336.1 GI: 15277243)

In some embodiments, provided herein are fusion proteins comprising a presenting peptide covalently linked to a β2M peptide via a linker, wherein the fusion proteins bind an MHC-like heavy chain to form an MHC-like complex that binds an inhibitory receptor of an immune cell to inhibit the immune cell. The MHC-like complex can be selected from the group consisting of CD1, MR1, FcRN and UL18.

In some embodiments, the MHC-like heavy chain is a CD1 heavy chain. Provided herein are fusion proteins comprising a CD1-restricted presenting molecule covalently linked to a β2M peptide via a linker, wherein the fusion protein binds a CD1 heavy chain to form a CD1 complex that binds an inhibitory receptor of an immune cell to inhibit the immune cell. In some embodiments, the fusion protein does not have a CD1 heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. In some embodiments, the MHC-like heavy chains include a CD1a heavy chain, a CD1b heavy chain, a CD1c heavy chain, a CD 1d heavy chain and a CD1e heavy chain. A human CD 1a heavy chain can have an amino acid sequence corresponding to the NCBI Reference Sequence NP_001754.2 (Accession: NP_001754.2 GI: 110618224). A human CD1b heavy chain can have an amino acid sequence corresponding to the NCBI Reference Sequence: XP_011508421.1 (Accession: XP_011508421.1 GI: 767910806). A human CD1c heavy chain can have an amino acid sequence corresponding to the NCBI Reference Sequence: XP_005245636.1 (Accession: XP_005245636.1 GI: 530365569). A human CD1d heavy chain can have an amino acid sequence corresponding to the NCBI Reference Sequence: NP_001358692.1 (Accession: NP_001358692.1 GI: 1707918971; precursor) A human CD1e heavy chain can have an amino acid sequence corresponding to the sequence having GenBank No. AAI31694.1 (Accession: AAI31694.1 GI: 124297103).

In some embodiments, the MHC-like heavy chain is an MR1 heavy chain. A human MRI heavy chain can have an amino acid sequence corresponding to the sequence having GenBank No. AAH12485.1 (Accession: AAH12485.1). Provided herein are fusion proteins comprising a MRI-restricted presenting molecule covalently linked to a β2M peptide via a linker, wherein the fusion proteins bind an MR1 heavy chain to form a MR1 complex that binds an inhibitory receptor of an immune cell to inhibit the immune cell. In some embodiments, the fusion protein does not have an MR1 heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. A human MR1 heavy chain can have an amino acid sequence corresponding to the sequence having GenBank No. CAB77667.1 (Accession: CAB77667.1 GI: 7271191).

In some embodiments, the MHC-like heavy chain is an FcRn heavy chain. Provided herein are fusion proteins comprising an FcRn-restricted presenting molecule covalently linked to a β2M peptide via a linker, wherein the fusion proteins bind an FcRn heavy chain to form a FcRn complex that binds an inhibitory receptor of an immune cell to inhibit the immune cell. In some embodiments, the fusion protein does not have an FcRn heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. A human FcRn heavy chain can have an amino acid sequence corresponding to the sequence having UniProtKB/Swiss-Prot: P55899.1 (Accession: P55899.1; GI: 2497331).

In some embodiments, the MHC-like heavy chain is UL18. Provided herein are fusion proteins comprising a UL18-restricted presenting molecule covalently linked to a β2M peptide via a linker, wherein the fusion proteins bind a UL18 to form a UL18 complex that binds an inhibitory receptor of an immune cell to inhibit the immune cell. In some embodiments, the fusion protein does not have a UL18 polypeptide. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. A human UL18 can have an amino acid sequence corresponding to the sequence having GenBank No. AJY57863.1 (Accession: AJY57863.1 GI: 777959438)

Provided herein are fusion proteins comprising a presenting peptide covalently linked to a β2M peptide via a linker. The presenting peptide can be derived from a virus, a prokaryote, a eukaryote, or a mammal. In some embodiments, the presenting peptide is derived from a virus. In some embodiments, the presenting peptide is derived from a prokaryote. In some embodiments, the presenting peptide is derived from a eukaryote. In some embodiments, the presenting peptide is derived from a mammal. In some embodiments, the presenting peptide is derived from a human. The presenting peptide can have 5-30, 5-25, 5-20, 7-20, 7-18, 7-15, 8-12, or 8-10 amino acids. In some embodiments, the presenting peptide has 5-30 amino acids. In some embodiments, the presenting peptide has 5-25 amino acids. In some embodiments, the presenting peptide has 5-20 amino acids. In some embodiments, the presenting peptide has 7-20 amino acids. In some embodiments, the presenting peptide has 7-18 amino acids. In some embodiments, the presenting peptide has 7-15 amino acids. In some embodiments, the presenting peptide has 8-12 amino acids. In some embodiments, the presenting peptide has 8-10 amino acids.

The presenting peptide can be a signal peptide of a Class I MHC molecule or a fragment thereof. In some embodiments, the presenting peptide is a fragment of a signal peptide of a Class I MHC molecule. In some embodiments, provided herein are fusion proteins comprising a presenting peptide covalently linked to a β2M peptide via a linker, wherein the presenting peptide is a signal peptide of a Class I MHC molecule, or a fragment thereof. In some embodiments, the fusion protein does not have an MHC heavy chain. In some embodiments, the fusion protein does not have an HLA-E heavy chain. In some embodiments, the fusion protein does not have an HLA-A heavy chain, an HLA-B heavy chain, an HLA-C heavy chain, an HLA-E heavy chain, an HLA-F heavy chain, or an HLA-G heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids.

The signal peptide of Class I MHC molecule is the peptide present at the N-terminus of a Class I MHC heavy chain, which guides and positions a newly synthesized Class I MHC heavy chain protein to the endoplasmic reticulum, allowing the mature heavy chain molecule, β2M and the presenting peptide to form a complex on the cell surface. The polypeptide is cleaved off in the mature protein. A signal peptide of the Class I MHC molecule generally has between 13-80 amino acids. Some signal peptides of the Class I MHC molecule have between 20-50 amino acids.

In some embodiments, the presenting peptide is a signal peptide of a Class I MHC molecule or a fragment thereof. The Class I MHC molecule can be selected from the group consisting of HLA-A1, HLA-A2, HLA-A*3401, HLA-A*80, HLA-B7, HLA-B*13, HLA-B15, HLA-Cw3, HLA-Cw*2, HLA-Cw*0809, HLA-Cw7, HLA-Cw*1701, HLA-G, and HLA-F. In some embodiments, the presenting peptide is a signal peptide or a fragment thereof of HLA-A1. In some embodiments, the presenting peptide is a signal peptide or a fragment thereof of HLA-A2. In some embodiments, the presenting peptide is a signal peptide or a fragment thereof of HLA-A*3401. In some embodiments, the presenting peptide is a signal peptide or a fragment thereof of HLA-A*80. In some embodiments, the presenting peptide is a signal peptide or a fragment thereof of HLA-B7. In some embodiments, the presenting peptide is a signal peptide or a fragment thereof of HLA- B*13. In some embodiments, the presenting peptide is a signal peptide or a fragment thereof of HLA-B15. In some embodiments, the presenting peptide is a signal peptide or a fragment thereof of HLA-Cw3. In some embodiments, the presenting peptide is a signal peptide or a fragment thereof of HLA-Cw*2. In some embodiments, the presenting peptide is a signal peptide or a fragment thereof of HLA-Cw*0809. In some embodiments, the presenting peptide is a signal peptide or a fragment thereof of HLA-Cw7. In some embodiments, the presenting peptide is a signal peptide or a fragment thereof of HLA-Cw*1701. In some embodiments, the presenting peptide is a signal peptide or a fragment thereof of HLA-G. In some embodiments, the presenting peptide is a signal peptide or a fragment thereof of HLA-F. In some embodiments, these exemplified signal peptides of a Class I MHC molecule or fragments thereof are HLA-E restricted presenting peptides. The presenting peptide that is a signal peptide of a Class I MHC molecule or a fragment thereof can have 5-30 amino acids. In some embodiments, the presenting peptide that is a signal peptide of a Class I MHC molecule or a fragment thereof can have 5-25 amino acids, 5-20 amino acids, 7-18 amino acids, 7-15 amino acids, 8-12 amino acids, or 8-10 amino acids. In some embodiments, the presenting peptide that is a fragment of a signal peptide of a Class I MHC molecule or a fragment thereof can have 8-10 amino acids.

In some embodiments, the presenting polypeptide is a signal peptide of a Class I MHC molecule or a fragment thereof, and has the following amino acid sequence: X₁X₂X₃X₄X₅X₆X₇X₈L; wherein X₁ is V or I; X₂ is T, A, M or L; X₃ is A, P, K or N; X₄ is P, L, or T; X₅ is R, Q or K; X₆ is T or A; X₇ is L, I, V or P; X_{g} is V, L, I, F or T (SEQ ID NO: 115).

In some embodiments, the presenting peptide is a signal peptide of Class I MHC molecule or a fragment thereof, and the amino acid sequence of the presenting peptide is selected from the group consisting of (1) YLLPRRGPRL (SEQ ID NO:21); (2) ALALVRMLI (SEQ ID NO:22); (3) AISPRTLNA (SEQ ID NO:23); (4) QMRPVSRVL (SEQ ID NO:24); (5) SQQPYLQLQ (SEQ ID NO:25); (6) VTAPRTLLL (SEQ ID NO:26); (7) VTAPRTVLL (SEQ ID NO:27); (8) VTAPRTLVL (SEQ ID NO:28); (9) VMAPQALLL (SEQ ID NO:29); (10) VMAPRALLL (SEQ ID NO:30); (11) VMAPRTLTL (SEQ ID NO:31); (12) VMAPRTLFL (SEQ ID NO:32); (13) VMAPRTLVL (SEQ ID NO:33); (14) VMAPRTLIL (SEQ ID NO:34); (15) IMAPRTLVL (SEQ ID NO:35); (16) VMAPRTLLL (SEQ ID NO:36); (17) VMPPRTLLL (SEQ ID NO:37); (18) VMAPRTVLL (SEQ ID NO:38); (19) VMAPRSLLL (SEQ ID NO:39); (20) VMAPRSLIL (SEQ ID NO:40); (21) VMTPRTLVL (SEQ ID NO:41); (22) VMAPRILIL (SEQ ID NO:42); (23) AMAPRTLIL (SEQ ID NO:43); (24) VIAPRTLVL (SEQ ID NO:44); (25) VMAPQSLLL (SEQ ID NO:45); (26) VMAPRTFVL (SEQ ID NO:46); (27) VMTPRTLIL (SEQ ID NO:47); (28) VTAPRTLIL (SEQ ID NO:48); (29) VMAPWTLLL (SEQ ID NO:49); (30) VMVPRSLIL (SEQ ID NO:50); (31) AMAPRTLVL (SEQ ID NO:51); (32) VIAPRTLIL (SEQ ID NO:52); (33) VIAPRTLLL (SEQ ID NO:53); (34) VLAPRTLIL (SEQ ID NO:54); (35) VMALRTLIL (SEQ ID NO:55); (36) VMAPRGLIL (SEQ ID NO:56); (37) VMAPRNLIL (SEQ ID NO:57); (38) VMAPRTLFV (SEQ ID NO:58); (39) VMAPRTLLM (SEQ ID NO:59); (40) VMAPRTLVM (SEQ ID NO:60); (41) VMAPRTSLL (SEQ ID NO:61); (42) VMAPRTSVL (SEQ ID NO:62); (43) VMAPWTLTL (SEQ ID NO:63); (44) VMAPWTLVL (SEQ ID NO:64); (45) VMDPRTLLL (SEQ ID NO:65); (46) VMGPRTLTL (SEQ ID NO:66); (47) VMGPRTLLL (SEQ ID NO:67); (48) VMVPQTLIL (SEQ ID NO:68); (49) VMVPRTLLL (SEQ ID NO:69); (50) VVAPRTLTL (SEQ ID NO:70); (51) VVAPRTLLL (SEQ ID NO:71); (52) VMVPRTLTL (SEQ ID NO:72); and (53) VMATRTLLL (SEQ ID NO:73). These exemplified presenting peptides are HLA-E restricted presenting peptides.

In some embodiments, the amino acid sequence of the presenting peptide is (1) YLLPRRGPRL (SEQ ID NO:21). In some embodiments, the amino acid sequence of the presenting peptide is (2) ALALVRMLI (SEQ ID NO:22). In some embodiments, the amino acid sequence of the presenting peptide is (3) AISPRTLNA (SEQ ID NO:23). In some embodiments, the amino acid sequence of the presenting peptide is (4) QMRPVSRVL (SEQ ID NO:24). In some embodiments, the amino acid sequence of the presenting peptide is (5) SQQPYLQLQ (SEQ ID NO:25). In some embodiments, the amino acid sequence of the presenting peptide is (6) VTAPRTLLL (SEQ ID NO:26). In some embodiments, the amino acid sequence of the presenting peptide is (7) VTAPRTVLL (SEQ ID NO:27). In some embodiments, the amino acid sequence of the presenting peptide is (8) VTAPRTLVL (SEQ ID NO:28). In some embodiments, the amino acid sequence of the presenting peptide is (9) VMAPQALLL (SEQ ID NO:29). In some embodiments, the amino acid sequence of the presenting peptide is (10) VMAPRALLL (SEQ ID NO:30). In some embodiments, the amino acid sequence of the presenting peptide is (11) VMAPRTLTL (SEQ ID NO:31). In some embodiments, the amino acid sequence of the presenting peptide is (12) VMAPRTLFL (SEQ ID NO:32). In some embodiments, the amino acid sequence of the presenting peptide is (13) VMAPRTLVL (SEQ ID NO:33). In some embodiments, the amino acid sequence of the presenting peptide is (14) VMAPRTLTL (SEQ ID NO:34). In some embodiments, the amino acid sequence of the presenting peptide is (15) IMAPRTLVL (SEQ ID NO:35). In some embodiments, the amino acid sequence of the presenting peptide is (16) VMAPRTLLL (SEQ ID NO:36). In some embodiments, the amino acid sequence of the presenting peptide is (17) VMPPRTLLL (SEQ ID NO:37). In some embodiments, the amino acid sequence of the presenting peptide is (18) VMAPRTVLL (SEQ ID NO:38). In some embodiments, the amino acid sequence of the presenting peptide is (19) VMAPRSLLL (SEQ ID NO:39). In some embodiments, the amino acid sequence of the presenting peptide is (20) VMAPRSLIL (SEQ ID NO:40). In some embodiments, the amino acid sequence of the presenting peptide is (21) VMTPRTLVL (SEQ ID NO:41). In some embodiments, the amino acid sequence of the presenting peptide is (22) VMAPRLTL (SEQ ID NO:42). In some embodiments, the amino acid sequence of the presenting peptide is (23) AMAPRTLTL (SEQ ID NO:43). In some embodiments, the amino acid sequence of the presenting peptide is (24) VIAPRTLVL (SEQ ID NO:44). In some embodiments, the amino acid sequence of the presenting peptide is (25) VMAPQSLLL (SEQ ID NO:45). In some embodiments, the amino acid sequence of the presenting peptide is (26) VMAPRTFVL (SEQ ID NO:46). In some embodiments, the amino acid sequence of the presenting peptide is (27) VMTPRTLIL (SEQ ID NO:47). In some embodiments, the amino acid sequence of the presenting peptide is (28) VTAPRTLTL (SEQ ID NO:48). In some embodiments, the amino acid sequence of the presenting peptide is (29) VMAPWTLLL (SEQ ID NO:49). In some embodiments, the amino acid sequence of the presenting peptide is (30) VMVPRSLIL (SEQ ID NO:50). In some embodiments, the amino acid sequence of the presenting peptide is (31) AMAPRTLVL (SEQ ID NO:51). In some embodiments, the amino acid sequence of the presenting peptide is (32) VIAPRTLIL (SEQ ID NO:52). In some embodiments, the amino acid sequence of the presenting peptide is (33) VIAPRTLLL (SEQ ID NO:53). In some embodiments, the amino acid sequence of the presenting peptide is (34) VLAPRTLTL (SEQ ID NO:54). In some embodiments, the amino acid sequence of the presenting peptide is (35) VMALRTLTL (SEQ ID NO:55). In some embodiments, the amino acid sequence of the presenting peptide is (36) VMAPRGLTL (SEQ ID NO:56). In some embodiments, the amino acid sequence of the presenting peptide is (37) VMAPRNLIL (SEQ ID NO:57). In some embodiments, the amino acid sequence of the presenting peptide is (38) VMAPRTLFV (SEQ ID NO:58). In some embodiments, the amino acid sequence of the presenting peptide is (39) VMAPRTLLM (SEQ ID NO:59). In some embodiments, the amino acid sequence of the presenting peptide is (40) VMAPRTLVM (SEQ ID NO:60). In some embodiments, the amino acid sequence of the presenting peptide is (41) VMAPRTSLL (SEQ ID NO:61). In some embodiments, the amino acid sequence of the presenting peptide is (42) VMAPRTSVL (SEQ ID NO:62). In some embodiments, the amino acid sequence of the presenting peptide is (43) VMAPWTLTL (SEQ ID NO:63). In some embodiments, the amino acid sequence of the presenting peptide is (44) VMAPWTLVL (SEQ ID NO:64). In some embodiments, the amino acid sequence of the presenting peptide is (45) VMDPRTLLL (SEQ ID NO:65). In some embodiments, the amino acid sequence of the presenting peptide is (46) VMGPRTLIL (SEQ ID NO:66). In some embodiments, the amino acid sequence of the presenting peptide is (47) VMGPRTLLL (SEQ ID NO:67). In some embodiments, the amino acid sequence of the presenting peptide is (48) VMVPQTLIL (SEQ ID NO:68). In some embodiments, the amino acid sequence of the presenting peptide is (49) VMVPRTLLL (SEQ ID NO:69). In some embodiments, the amino acid sequence of the presenting peptide is (50) VVAPRTLIL (SEQ ID NO:70). In some embodiments, the amino acid sequence of the presenting peptide is (51) VVAPRTLLL (SEQ ID NO:71). In some embodiments, the amino acid sequence of the presenting peptide is (52) VMVPRTLIL (SEQ ID NO:72). In some embodiments, the amino acid sequence of the presenting peptide is (53) VMATRTLLL (SEQ ID NO:73).

Additional HLA-E restricted presenting peptides are also described in the art. See e.g. United States Patent Application 20190314445; Celik et al., Immunogenetics 2016. 68:29-41; Hannoun et al. Immunology Letters 2018. 202:65-72; Rolle et al. Cell Rep 2018. 14(8): 1967-1976; Rolle et al. Front Immunol 2018. 9:2410. All of these references are hereby incorporated by reference in their entireties. Similarly, presenting peptides that are restricted to HLA-A, HLA-B, HLA-C, HLA-F, or HLA-G are also well known in the art. See e.g. Gfeller et al. Front Immunol 2018. 9:1716; Luo et al. Bioinform Biol Insights 2015. 9(Suppl 3):21-9; You et al. PATTERN RECOGNITION IN BIOINFORMATICS 2007. pp 337-348. Lecture Notes in Computer Science, vol 4774. Springer, Berlin, Heidelberg; Purcell et al. Nat Protoc 2019. 14(6):1687-1707; Rock et al. Trends Immunol 2016. 37(11):724-737; Celik et al. Immunogenetics 2018. 70(8): 485-494; Di Marco et al. J Immunol 2017. 199(8):2639-2651; Dulberger et al. Immunity 2017. 46(6):1018-1029.e7; Ho et al. Immunogenetics 2019. 71(5-6):353-360; Sim et al. Immunity 2017. 46(6):972-974; Rammensee et al. Annu Rev Immunol 1993. 11:213-44; Schellens et al. PLoS One 2015. 10(9):e0136417. All of these references are hereby incorporated by reference in their entireties.

In some embodiments, the presenting polypeptide can also be derived from a virus protein, such as of CMV, EBV, HIV, etc. In some embodiments, the presenting peptide is a fragment of CMV. In some embodiments, the presenting peptide is a fragment of EBV. In some embodiments, the presenting peptide is a fragment of HIV.

The fusion proteins provided herein have a presenting peptide covalently linked to a β2M peptide via a linker. In some embodiments, the fusion proteins provided herein have the presenting peptide, the linker, and the β2M peptide from N-terminus to C-terminus. In some embodiments, the fusion proteins provided herein have the β2M peptide, the linker, and the presenting peptide, from N-terminus to C-terminus. In some embodiments, the fusion proteins disclosed herein further include a membrane localization signal peptide at its N-terminus, which guides the fusion protein to locate to the cell membrane and is subsequently cleaved off the fusion protein. Membrane localization signal peptides can be between about 20-80 amino acids in length. Exemplary membrane localization signal peptide can have the amino acid sequence of SEQ ID NO:3 or SEQ ID NO:4. In some embodiments, the fusion proteins provided herein consist of the presenting peptide, the linker, and the β2M peptide from N-terminus to C-terminus. In some embodiments, the fusion proteins provided herein consist of the β2M peptide, the linker, and the presenting peptide from N-terminus to C-terminus.

The fusion proteins provided herein have a presenting peptide covalently linked to a β2M peptide via a linker. The linker links the presenting peptide and β2M peptide of the fusion protein, and provides sufficient flexibility such that the fusion protein can form a stable complex with an MHC heavy chain. The linker provide herein can have about 5 to 50 amino acids, and contains at least one of the following five amino acids (Gly, Ala, Pro, Val, and Leu), and at least one of the eight amino acids (Ser, Thr, Glu, Lys, Asn, Gln, Asp, and Arg). In some embodiments, the linker provided herein has between 5 and 30 amino acids.

In some embodiments, the amino acid sequence of the linker is (EAAAK)n, n=1, 2, 3, 4 or 5 (SEQ ID NO:109). In some embodiments, the amino acid sequence of the linker is (EAAAK)n, n=3, 4 or 5 (SEQ ID NO:110). In some embodiments, the amino acid sequence of the linker is (GGGGS)n, n=1, 2, 3, 4 or 5 (SEQ ID NO:111). In some embodiments, the amino acid sequence of the linker is (GGGGS)n, n=3, 4 or 5 (SEQ ID NO:112).

In some embodiments, the amino acid sequence of the linker is (G)nS, n=1, 2, 3, 4 or 5 (SEQ ID NO:113). In some embodiments, the amino acid sequence of the linker is an m series combination of (G)nS, n=1, 2, 3, 4 or 5; and m=1, 2, 3, 4 or 5 (SEQ ID NO:122). For example, the amino acid sequence of the linker can be (G4S)(G2S)(GS)(G3S), namely, GGGGSGGSGSGGGS (SEQ ID NO:114).

In some embodiments, the amino acid sequence of the linker is (GGGGS)3, or (G4S)3, namely, GGGGSGGGGSGGGGS (SEQ ID NO:1). In some embodiments, the amino acid sequence of the linker is (G4S)4 sequence, namely, GGGGSGGGGSGGGGSGGGGS (SEQ ID NO:2).

The fusion proteins provided herein have a presenting peptide covalently linked to a β2M peptide via a linker. In some embodiments, the β2M peptide is a wild type β2M protein. In some embodiments, the β2M peptide is wild type human β2M (SEQ ID NO:7). In some embodiments, the β2M peptide is a functional fragment of a wild type β2M protein. In some embodiments, the β2M peptide is a functional variant of a wild type β2M protein. In some embodiments, the β2M peptide is a functional fragment of wild type human β2M. In some embodiments, the β2M peptide is a functional variant of wild type human β2M. In some embodiments, the β2M peptide is a functional variant of a wild type β2M protein having an amino acid sequence that is at least 85%, at least 90%, at least 95%, at least 98%, or at least 99% identical to SEQ ID NO:7. In some embodiments, the amino acid sequence of the β2M peptide is at least 85% identical to SEQ ID NO:7. In some embodiments, the amino acid sequence of the β2M peptide is at least 90% identical to SEQ ID NO:7. In some embodiments, the amino acid sequence of the β2M peptide is at least 95% identical to SEQ ID NO:7. In some embodiments, the amino acid sequence of the β2M peptide is at least 98% identical to SEQ ID NO:7. In some embodiments, the amino acid sequence of the β2M peptide is at least 99% identical to SEQ ID NO:7. In some embodiments, the amino acid sequence of the β2M peptide is SEQ ID NO:7.

In some embodiments, the β2M peptide is the mature form of human β2M (SEQ ID NO:81), and the fusion proteins provided herein have a presenting peptide covalently linked to the mature human β2M peptide via a linker. In In some embodiments, the β2M peptide is a functional fragment of the mature form of human β2M protein. In some embodiments, the β2M peptide is a functional variant of the mature form of human β2M protein. In some embodiments, the β2M peptide is a functional variant of the mature form of human β2M protein having an amino acid sequence that is at least 85%, at least 90%, at least 95%, at least 98%, or at least 99% identical to SEQ ID NO:81. In some embodiments, the amino acid sequence of the β2M peptide is at least 85% identical to SEQ ID NO:81. In some embodiments, the amino acid sequence of the β2M peptide is at least 90% identical to SEQ ID NO:81. In some embodiments, the amino acid sequence of the β2M peptide is at least 95% identical to SEQ ID NO:81. In some embodiments, the amino acid sequence of the β2M peptide is at least 98% identical to SEQ ID NO:81. In some embodiments, the amino acid sequence of the β2M peptide is at least 99% identical to SEQ ID NO:81. In some embodiments, the amino acid sequence of the β2M peptide is SEQ ID NO:81.

In some embodiments, the amino acid sequence of the fusion protein provided herein comprises an amino acid sequence that is at least 85%, at least 90%, at least 95%, at least 98%, or at least 99% identical to an amino acid sequence selected from the group consisting of SEQ ID NOs: 5 and 13-18. In some embodiments, the amino acid sequence of the fusion protein provided herein comprises an amino acid sequence selected from the group consisting of SEQ ID NOs: 5 and 13-18. In some embodiments, the fusion protein does not have an MHC heavy chain. In some embodiments, the fusion protein does not have an HLA-E heavy chain. In some embodiments, the fusion protein does not have an HLA-A heavy chain, an HLA-B heavy chain, an HLA-C heavy chain, an HLA-E heavy chain, an HLA-F heavy chain, or an HLA-G heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. In some embodiments, the fusion protein has about 120 to about 180 amino acids.

In some embodiments, the amino acid sequence of the fusion protein provided herein comprises an amino acid sequence that is at least 85% identical to SEQ ID NO:5 or amino acids 21-143 of SEQ ID NO:5. In some embodiments, the amino acid sequence of the fusion protein provided herein comprises an amino acid sequence that is at least 90% identical to SEQ ID NO:5 or amino acids 21-143 of SEQ ID NO:5. In some embodiments, the amino acid sequence of the fusion protein provided herein comprises an amino acid sequence that is at least 95% identical to SEQ ID NO:5 or amino acids 21-143 of SEQ ID NO:5. In some embodiments, the amino acid sequence of the fusion protein provided herein comprises an amino acid sequence that is at least 98% identical to SEQ ID NO:5 or amino acids 21-143 of SEQ ID NO:5. In some embodiments, the amino acid sequence of the fusion protein provided herein comprises an amino acid sequence that is at least 99% identical to SEQ ID NO:5 or amino acids 21-143 of SEQ ID NO:5. In some embodiments, the amino acid sequence of the fusion protein provided herein comprises SEQ ID NO:5. In some embodiments, the amino acid sequence of the fusion protein provided herein comprises amino acids 21-143 of SEQ ID NO:5.

In some embodiments, the amino acid sequence of the fusion protein provided herein comprises an amino acid sequence that is at least 85% identical to SEQ ID NO: 13 or amino acids 21-143 of SEQ ID NO:13. In some embodiments, the amino acid sequence of the fusion protein provided herein comprises an amino acid sequence that is at least 90% identical to SEQ ID NO:13 or amino acids 21-143 of SEQ ID NO:13. In some embodiments, the amino acid sequence of the fusion protein provided herein comprises an amino acid sequence that is at least 95% identical to SEQ ID NO: 13 or amino acids 21-143 of SEQ ID NO:13. In some embodiments, the amino acid sequence of the fusion protein provided herein comprises an amino acid sequence that is at least 98% identical to SEQ ID NO: 13 or amino acids 21-143 of SEQ ID NO:13. In some embodiments, the amino acid sequence of the fusion protein provided herein comprises an amino acid sequence that is at least 99% identical to SEQ ID NO: 13 or amino acids 21-143 of SEQ ID NO:13. In some embodiments, the amino acid sequence of the fusion protein provided herein comprises SEQ ID NO:13. In some embodiments, the amino acid sequence of the fusion protein provided herein comprises amino acids 21-143 of SEQ ID NO:13.

In some embodiments, the amino acid sequence of the fusion protein provided herein comprises an amino acid sequence that is at least 85% identical to SEQ ID NO: 14 or amino acids 21-143 of SEQ ID NO:14. In some embodiments, the amino acid sequence of the fusion protein provided herein comprises an amino acid sequence that is at least 90% identical to SEQ ID NO:14 or amino acids 21-143 of SEQ ID NO:14. In some embodiments, the amino acid sequence of the fusion protein provided herein comprises an amino acid sequence that is at least 95% identical to SEQ ID NO: 14 or amino acids 21-143 of SEQ ID NO:14. In some embodiments, the amino acid sequence of the fusion protein provided herein comprises an amino acid sequence that is at least 98% identical to SEQ ID NO: 14 or amino acids 21-143 of SEQ ID NO:14. In some embodiments, the amino acid sequence of the fusion protein provided herein comprises an amino acid sequence that is at least 99% identical to SEQ ID NO:14 or amino acids 21-143 of SEQ ID NO:14. In some embodiments, the amino acid sequence of the fusion protein provided herein comprises SEQ ID NO:14. In some embodiments, the amino acid sequence of the fusion protein provided herein comprises amino acids 21-143 of SEQ ID NO:14.

In some embodiments, the amino acid sequence of the fusion protein provided herein comprises an amino acid sequence that is at least 85% identical to SEQ ID NO:15 or amino acids 21-143 of SEQ ID NO:15. In some embodiments, the amino acid sequence of the fusion protein provided herein comprises an amino acid sequence that is at least 90% identical to SEQ ID NO:15 or amino acids 21-143 of SEQ ID NO:15. In some embodiments, the amino acid sequence of the fusion protein provided herein comprises an amino acid sequence that is at least 95% identical to SEQ ID NO: 15 or amino acids 21-143 of SEQ ID NO:15. In some embodiments, the amino acid sequence of the fusion protein provided herein comprises an amino acid sequence that is at least 98% identical to SEQ ID NO: 15 or amino acids 21-143 of SEQ ID NO:15. In some embodiments, the amino acid sequence of the fusion protein provided herein comprises an amino acid sequence that is at least 99% identical to SEQ ID NO:15 or amino acids 21-143 of SEQ ID NO:15. In some embodiments, the amino acid sequence of the fusion protein provided herein comprises SEQ ID NO:15. In some embodiments, the amino acid sequence of the fusion protein provided herein comprises amino acids 21-143 of SEQ ID NO:15.

In some embodiments, the amino acid sequence of the fusion protein provided herein comprises an amino acid sequence that is at least 85% identical to SEQ ID NO: 16 or amino acids 21-143 of SEQ ID NO:16. In some embodiments, the amino acid sequence of the fusion protein provided herein comprises an amino acid sequence that is at least 90% identical to SEQ ID NO:16 or amino acids 21-143 of SEQ ID NO:16. In some embodiments, the amino acid sequence of the fusion protein provided herein comprises an amino acid sequence that is at least 95% identical to SEQ ID NO: 16 or amino acids 21-143 of SEQ ID NO:16. In some embodiments, the amino acid sequence of the fusion protein provided herein comprises an amino acid sequence that is at least 98% identical to SEQ ID NO: 16 or amino acids 21-143 of SEQ ID NO:16. In some embodiments, the amino acid sequence of the fusion protein provided herein comprises an amino acid sequence that is at least 99% identical to SEQ ID NO:16 or amino acids 21-143 of SEQ ID NO:16. In some embodiments, the amino acid sequence of the fusion protein provided herein comprises SEQ ID NO:16. In some embodiments, the amino acid sequence of the fusion protein provided herein comprises amino acids 21-143 of SEQ ID NO:16.

In some embodiments, the amino acid sequence of the fusion protein provided herein comprises an amino acid sequence that is at least 85% identical to SEQ ID NO: 17 or amino acids 21-143 of SEQ ID NO:17. In some embodiments, the amino acid sequence of the fusion protein provided herein comprises an amino acid sequence that is at least 90% identical to SEQ ID NO:17 or amino acids 21-143 of SEQ ID NO:17. In some embodiments, the amino acid sequence of the fusion protein provided herein comprises an amino acid sequence that is at least 95% identical to SEQ ID NO: 17 or amino acids 21-143 of SEQ ID NO:17. In some embodiments, the amino acid sequence of the fusion protein provided herein comprises an amino acid sequence that is at least 98% identical to SEQ ID NO: 17 or amino acids 21-143 of SEQ ID NO:17. In some embodiments, the amino acid sequence of the fusion protein provided herein comprises an amino acid sequence that is at least 99% identical to SEQ ID NO: 17 or amino acids 21-143 of SEQ ID NO:17. In some embodiments, the amino acid sequence of the fusion protein provided herein comprises SEQ ID NO:17. In some embodiments, the amino acid sequence of the fusion protein provided herein comprises amino acids 21-143 of SEQ ID NO:17.

In some embodiments, the amino acid sequence of the fusion protein provided herein comprises an amino acid sequence that is at least 85% identical to SEQ ID NO:18 or amino acids 21-144 of SEQ ID NO:18. In some embodiments, the amino acid sequence of the fusion protein provided herein comprises an amino acid sequence that is at least 90% identical to SEQ ID NO:18 or amino acids 21-144 of SEQ ID NO:18. In some embodiments, the amino acid sequence of the fusion protein provided herein comprises an amino acid sequence that is at least 95% identical to SEQ ID NO: 18 or amino acids 21-144 of SEQ ID NO:18. In some embodiments, the amino acid sequence of the fusion protein provided herein comprises an amino acid sequence that is at least 98% identical to SEQ ID NO: 18 or amino acids 21-144 of SEQ ID NO:18. In some embodiments, the amino acid sequence of the fusion protein provided herein comprises an amino acid sequence that is at least 99% identical to SEQ ID NO:18 or amino acids 21-144 of SEQ ID NO:18. In some embodiments, the amino acid sequence of the fusion protein provided herein comprises SEQ ID NO:18. In some embodiments, the amino acid sequence of the fusion protein provided herein comprises amino acids 21-144 of SEQ ID NO:18.

In some embodiments, the amino acid sequence of the fusion protein provided herein consists of an amino acid sequence that is at least 85%, at least 90%, at least 95%, at least 98%, or at least 99% identical to an amino acid sequence selected from the group consisting of SEQ ID NOs: 5 and 13-18. In some embodiments, the amino acid sequence of the fusion protein provided herein consists of an amino acid sequence selected from the group consisting of SEQ ID NOs: 5 and 13-18.

In some embodiments, the amino acid sequence of the fusion protein provided herein is at least 85% identical to SEQ ID NO:5 or amino acids 21-143 of SEQ ID NO:5. In some embodiments, the amino acid sequence of the fusion protein provided herein is at least 90% identical to SEQ ID NO:5 or amino acids 21-143 of SEQ ID NO:5. In some embodiments, the amino acid sequence of the fusion protein provided herein is at least 95% identical to SEQ ID NO:5 or amino acids 21-143 of SEQ ID NO:5. In some embodiments, the amino acid sequence of the fusion protein provided herein is at least 98% identical to SEQ ID NO:5 or amino acids 21-143 of SEQ ID NO:5. In some embodiments, the amino acid sequence of the fusion protein provided herein is at least 99% identical to SEQ ID NO:5 or amino acids 21-143 of SEQ ID NO:5. In some embodiments, the amino acid sequence of the fusion protein provided herein consists of SEQ ID NO:5. In some embodiments, the amino acid sequence of the fusion protein provided herein consists of amino acids 21-143 of SEQ ID NO:5.

In some embodiments, the amino acid sequence of the fusion protein provided herein is at least 85% identical to SEQ ID NO:13 or amino acids 21-143 of SEQ ID NO:13. In some embodiments, the amino acid sequence of the fusion protein provided herein is at least 90% identical to SEQ ID NO: 13 or amino acids 21-143 of SEQ ID NO:13. In some embodiments, the amino acid sequence of the fusion protein provided herein is at least 95% identical to SEQ ID NO:13 or amino acids 21-143 of SEQ ID NO:13. In some embodiments, the amino acid sequence of the fusion protein provided herein is at least 98% identical to SEQ ID NO:13 or amino acids 21-143 of SEQ ID NO:13. In some embodiments, the amino acid sequence of the fusion protein provided herein is at least 99% identical to SEQ ID NO: 13 or amino acids 21-143 of SEQ ID NO:13. In some embodiments, the amino acid sequence of the fusion protein provided herein consists of SEQ ID NO: 13. In some embodiments, the amino acid sequence of the fusion protein provided herein consists of amino acids 21-143 of SEQ ID NO:13.

In some embodiments, the amino acid sequence of the fusion protein provided herein is at least 85% identical to SEQ ID NO:14 or amino acids 21-143 of SEQ ID NO:14. In some embodiments, the amino acid sequence of the fusion protein provided herein is at least 90% identical to SEQ ID NO: 14 or amino acids 21-143 of SEQ ID NO:14. In some embodiments, the amino acid sequence of the fusion protein provided herein is at least 95% identical to SEQ ID NO:14 or amino acids 21-143 of SEQ ID NO:14. In some embodiments, the amino acid sequence of the fusion protein provided herein is at least 98% identical to SEQ ID NO:14 or amino acids 21-143 of SEQ ID NO:14. In some embodiments, the amino acid sequence of the fusion protein provided herein is at least 99% identical to SEQ ID NO: 14 or amino acids 21-143 of SEQ ID NO:14. In some embodiments, the amino acid sequence of the fusion protein provided herein consists of SEQ ID NO: 14. In some embodiments, the amino acid sequence of the fusion protein provided herein consists of amino acids 21-143 of SEQ ID NO:14.

In some embodiments, the amino acid sequence of the fusion protein provided herein is at least 85% identical to SEQ ID NO:15 or amino acids 21-143 of SEQ ID NO:15. In some embodiments, the amino acid sequence of the fusion protein provided herein is at least 90% identical to SEQ ID NO: 15 or amino acids 21-143 of SEQ ID NO:15. In some embodiments, the amino acid sequence of the fusion protein provided herein is at least 95% identical to SEQ ID NO:15 or amino acids 21-143 of SEQ ID NO:15. In some embodiments, the amino acid sequence of the fusion protein provided herein is at least 98% identical to SEQ ID NO:15 or amino acids 21-143 of SEQ ID NO:15. In some embodiments, the amino acid sequence of the fusion protein provided herein is at least 99% identical to SEQ ID NO:15 or amino acids 21-143 of SEQ ID NO:15. In some embodiments, the amino acid sequence of the fusion protein provided herein consists of SEQ ID NO: 15. In some embodiments, the amino acid sequence of the fusion protein provided herein consists of amino acids 21-143 of SEQ ID NO:15.

In some embodiments, the amino acid sequence of the fusion protein provided herein is at least 85% identical to SEQ ID NO:16 or amino acids 21-143 of SEQ ID NO:16. In some embodiments, the amino acid sequence of the fusion protein provided herein is at least 90% identical to SEQ ID NO: 16 or amino acids 21-143 of SEQ ID NO:16. In some embodiments, the amino acid sequence of the fusion protein provided herein is at least 95% identical to SEQ ID NO:16 or amino acids 21-143 of SEQ ID NO:16. In some embodiments, the amino acid sequence of the fusion protein provided herein is at least 98% identical to SEQ ID NO:16 or amino acids 21-143 of SEQ ID NO:16. In some embodiments, the amino acid sequence of the fusion protein provided herein is at least 99% identical to SEQ ID NO: 16 or amino acids 21-143 of SEQ ID NO:16. In some embodiments, the amino acid sequence of the fusion protein provided herein consists of SEQ ID NO: 16. In some embodiments, the amino acid sequence of the fusion protein provided herein consists of amino acids 21-143 of SEQ ID NO:16.

In some embodiments, the amino acid sequence of the fusion protein provided herein is at least 85% identical to SEQ ID NO:17 or amino acids 21-143 of SEQ ID NO:17. In some embodiments, the amino acid sequence of the fusion protein provided herein is at least 90% identical to SEQ ID NO: 17 or amino acids 21-143 of SEQ ID NO:16. In some embodiments, the amino acid sequence of the fusion protein provided herein is at least 95% identical to SEQ ID NO:17 or amino acids 21-143 of SEQ ID NO:16. In some embodiments, the amino acid sequence of the fusion protein provided herein is at least 98% identical to SEQ ID NO:17 or amino acids 21-143 of SEQ ID NO:16. In some embodiments, the amino acid sequence of the fusion protein provided herein is at least 99% identical to SEQ ID NO: 17 or amino acids 21-143 of SEQ ID NO:16. In some embodiments, the amino acid sequence of the fusion protein provided herein consists of SEQ ID NO: 17. In some embodiments, the amino acid sequence of the fusion protein provided herein consists of amino acids 21-143 of SEQ ID NO:17.

In some embodiments, the amino acid sequence of the fusion protein provided herein is at least 85% identical to SEQ ID NO:18 or amino acids 21-144 of SEQ ID NO:18. In some embodiments, the amino acid sequence of the fusion protein provided herein is at least 90% identical to SEQ ID NO: 18 or amino acids 21-144 of SEQ ID NO:18. In some embodiments, the amino acid sequence of the fusion protein provided herein is at least 95% identical to SEQ ID NO:18 or amino acids 21-144 of SEQ ID NO:18. In some embodiments, the amino acid sequence of the fusion protein provided herein is at least 98% identical to SEQ ID NO:18 or amino acids 21-144 of SEQ ID NO:18. In some embodiments, the amino acid sequence of the fusion protein provided herein is at least 99% identical to SEQ ID NO:18 or amino acids 21-144 of SEQ ID NO:18. In some embodiments, the amino acid sequence of the fusion protein provided herein consists of SEQ ID NO: 18. In some embodiments, the amino acid sequence of the fusion protein provided herein consists of amino acids 21-144 of SEQ ID NO:18.

The fusion proteins described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press; Borrebaeck (ed.) (1995) Antibody Engineering, Second Edition, Oxford University Press; Lo (ed.) (2006) Antibody Engineering: Methods and Protocols (Methods in Molecular Biology); Vol. 248, Humana Press, Inc; each of which is incorporated herein by reference in its entirety.

The fusion proteins described herein can be produced and isolated using methods known in the art. Peptides can be synthesized, in whole or in part, using chemical methods (see, e.g., Caruthers (1980). Nucleic Acids Res. Symp. Ser. 215; Horn (1980); and Banga, A. K., Therapeutic Peptides and Proteins, Formulation, Processing and Delivery Systems (1995) Technomic Publishing Co., Lancaster, PA). Peptide synthesis can be performed using various solid-phase techniques (see, e.g., Roberge Science 269:202 (1995); Merrifield, Methods. Enzymol. 289:3 (1997)) and automated synthesis may be achieved, e.g., using the ABI 431A Peptide Synthesizer (Perkin Elmer) in accordance with the manufacturer's instructions. Peptides can also be synthesized using combinatorial methodologies. Synthetic residues and polypeptides can be synthesized using a variety of procedures and methodologies known in the art (see, e.g., Organic Syntheses Collective Volumes, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce invention peptide sequences, variants, fusions and chimeras, and variations, derivatives, substitutions and modifications thereof.

In some embodiments, a recombinant expression vector is used to amplify and express the nucleic acid encoding a fusion protein disclosed herein. For example, a recombinant expression vector can be a replicable DNA construct that includes synthetic or cDNA-derived DNA fragments encoding a fusion protein, operatively linked to suitable transcriptional and/or translational regulatory elements derived from mammalian, microbial, viral or insect genes. In some embodiments, a viral vector is used. DNA regions are operatively linked when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue.

A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

In some embodiments, a fusion protein of the present disclosure is expressed from one or more vectors. Suitable host cells for expression of a fusion protein include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production are well-known in the art.

Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

### 6.3 Synthetic Receptors

In some embodiments, a fusion protein provided herein can be co-expressed with a synthetic receptor in a cell. In some embodiments, a fusion protein provided herein can be conjugated to a synthetic receptor. As used herein, the term “synthetic receptor” refers to an engineered cell surface protein or protein complex comprising (1) a target-binding domain that can specifically bind a target molecule, and (2) a functional domain that can activate a signaling pathway in the engineered cell. The target-binding domain comprises an extracellular domain. The functional domain comprises an intracellular domain. The synthetic receptor further includes a transmembrane sequence. The synthetic receptor can be a protein complex that comprises proteins expressed from exogenous nucleic acids. The synthetic receptor can also be a protein complex that comprises at least one protein that is exogenously expressed, and at least one protein that is endogenously expressed. In some embodiments, the engineered cell can be an immune cell, such as a T cell, a national killer (NK) cell, a B cell, a macrophage, etc., and the functional domain can activate the immune cell, either directly or indirectly. The synthetic receptor can be selected from the group consisting of: a chimeric antigen receptor (“CAR”), a T cell receptor (“TCR”), a TCR receptor fusion construct (“TRuC”), a T cell antigen coupler (“TAC”), an antibody TCR receptor (“AbTCR”), and a chimeric CD3F receptor. In some embodiments, the synthetic receptor is a CAR. In some embodiments, the synthetic receptor is a TCR. In some embodiments, the synthetic receptor is a TRuC. In some embodiments, the synthetic receptor is a TAC. In some embodiments, the synthetic receptor is an AbTCR. In some embodiments, the synthetic receptor is a chimeric CD3 receptor.

### 6.3.1 Target-Binding Domains

The synthetic receptors provided herein include a target-binding domain. The target-binding domain is an extracellular domain that can bind a target molecule. In some embodiments, the target molecule is an antigen on a target tissue. In some embodiments, the target molecule is a viral antigen. In some embodiments, the target molecule is a cancer antigen. In some embodiments, the target-binding domain of a synthetic receptor provided herein can also be an epitope that can be recognized by an antibody (e.g. a bispecific or multispecific antibody) that can bind a target molecule on a target tissue, such as a cancer antigen.

Such an antigen binding domain is generally derived from an antibody. In one embodiment, the antigen binding domain can be an scFv or an Fab, or any suitable antigen binding fragment of an antibody (see Sadelain et al., Cancer Discov. 3:388-398 (2013)). In some embodiments, the extracellular antigen-binding domain is an scFv. Many antibodies or antigen binding domains derived from antibodies that bind to a cancer antigen are known in the art. Alternatively, such antibodies or antigen binding domains can be produced by routine methods. Methods of generating an antibody are well known in the art, including methods of producing a monoclonal antibody or screening a library to obtain an antigen binding polypeptide, including screening a library of human Fabs (Winter and Harris, Immunol. Today 14:243-246 (1993); Ward et al., Nature 341:544-546 (1989); Harlow and Lane, Antibodies: A Laboratory Manual, Cold Spring Harbor Laboratory Press (1988); Hilyard et al., Protein Engineering: A practical approach (IRL Press 1992); Borrabeck, Antibody Engineering, 2nd ed. (Oxford University Press 1995); Huse et al., Science 246:1275-1281 (1989)).

The antigen binding domain derived from an antibody can be human, humanized, chimeric, CDR-grafted, and the like, as desired. For example, if a mouse monoclonal antibody is a source antibody for generating the antigen binding domain, such an antibody can be humanized by grafting CDRs of the mouse antibody onto a human framework (see Borrabeck, supra, 1995), which can be beneficial for administering the synthetic receptor to a human subject. In a preferred embodiment, the antigen binding domain is an scFv. The generation of scFvs is well known in the art (see, for example, Huston, et al., Proc. Nat. Acad. Sci. USA 85:5879-5883 (1988); Ahmad et al., Clin. Dev. Immunol. 2012: ID980250 (2012); U.S. Pat. Nos. 5,091,513, 5,132,405 and 4,956,778; and U.S. Patent Publication Nos. 20050196754 and 20050196754)).

With respect to obtaining a cancer antigen binding activity, one skilled in the art can readily obtain a suitable cancer antigen binding activity, such as an antibody, using any of the well-known methods for generating and screening for an antibody that binds to a desired antigen, as disclosed herein, including the generation of an scFv that binds to a cancer antigen, which is particularly useful in a synthetic receptor as disclosed herein. In addition, a number cancer antigen antibodies, in particular monoclonal antibodies, are commercially available and can also be used as a source for a cancer antigen binding activity, such as an scFv, to generate a synthetic receptor.

In some embodiments, the target-binding domain can be an antibody fragment, derivative or mimetic thereof, where these fragments, derivatives and mimetics have the requisite binding affinity for the target molecule. Such antibody fragments or derivatives generally include at least the V_{H} and V_{L} domains of the subject antibodies, so as to retain the binding characteristics of the subject antibodies. An antibody fragment as used herein refers to a molecule other than an intact antibody that comprises a portion of an antibody and generally an antigen-binding site. Examples of antibody fragments include, but are not limited to, Fab, Fab′, F(ab′)2, Fv, single chain antibody molecules (e.g., scFv), disulfide-linked scFv (dsscFv), diabodies, tribodies, tetrabodies, minibodies, dual variable domain antibodies (DVD), single variable domain antibodies (e.g., camelid antibodies, alpaca antibodies), single variable domain of heavy chain antibodies (VHH), nanobodies, and multispecific antibodies formed from antibody fragments. In some embodiments, the target-binding domain is an Fab. In some embodiments, the target-binding domain is a scFv. In some embodiments, the target-binding domain comprises a single variable domain antibody.

In some embodiments, the target-binding domain comprises an antibody mimetic. An antibody mimetic can be molecules that, like antibodies, can specifically bind antigens, but that are not structurally related to antibodies. The antibody mimetics are usually artificial peptides within a molar mass of about 2 to 20 kDa. Nucleic acids and small molecules are sometimes considered antibody mimetics as well. Antibody mimetics known in the art include adnectins, affibodies, affilins, affimers, affitins, alphabodies, anticalins, aptamers, avimers, bicyclic peptides, Centyrin (J&J), DARPins, Fynomers, Knottins, Kunitz domain peptides, monobodies, and nanoCLAMPs.

In some embodiments, the target-binding domain comprises antibody-like scaffolds (e.g. Owens, Nature Biotechnology 35: 602-603(2017); Simeon and Chen, Protein Cell 9(1): 3-14 (2018)).

Alternatively to using an antigen binding domain derived from an antibody, a target-binding domain of a synthetic receptor can comprise a ligand or extracellular ligand binding domain of a receptor (see Sadelain et al., Cancer Discov. 3:388-398 (2013); Sharpe et al., Dis. Model Mech. 8:337-350 (2015)). In this case, the ligand or extracellular ligand binding domain of a receptor provides to the synthetic receptor the ability to target the cell expressing the synthetic receptor to the corresponding receptor or ligand. The ligand or extracellular ligand binding domain is selected such that the cell expressing the synthetic receptor is targeted to a cancer cell or tumor (see Sadelain et al., Cancer Discov. 3:388-398 (2013); Sharpe et al., Dis. Model Mech. 8:337-350 (2015), and references cited therein). In an embodiment, the ligand or extracellular ligand binding domain is selected to bind to a cancer antigen that is the corresponding receptor or ligand (see Sadelain et al., Cancer Discov. 3:388-398 (2013)).

Additionally, in some embodiments, a target-binding domain of a synthetic receptor can comprise a chimeric autoantibody receptor (CAAR), namely, an autoantigen that can be recognized by a B cell receptor (BCR) (see Ellebrecht et al. Science 353(6295), 179-184 (2016)).

For a synthetic receptor directed to a cancer antigen, the antigen binding domain of the synthetic receptor is selected to bind to an antigen expressed on a cancer cell. Such a cancer antigen can be uniquely expressed on a cancer cell, or the cancer antigen can be overexpressed in a cancer cell relative to noncancerous cells or tissues. The cancer antigen to be bound by the synthetic receptor is chosen to provide targeting of the cell expressing the synthetic receptor over noncancerous cells or tissues.

In some embodiments, the synthetic receptor comprises an antigen-binding domain that specifically binds a viral antigen. In some embodiments, the viral antigen is EBV. In some embodiments, the viral antigen is HPV. It is understood that these or other viral antigens can be utilized for targeting by a synthetic receptor disclosed herein.

The cancer antigen can be a tumor antigen. Any suitable cancer antigen can be chosen based on the type of cancer exhibited by a subject (cancer patient) to be treated. It is understood that the selected cancer antigen is expressed in a manner such that the cancer antigen is accessible for binding by the synthetic receptor. Generally, the cancer antigen to be targeted by a cell expressing a synthetic receptor is expressed on the cell surface of a cancer cell. However, it is understood that any cancer antigen that is accessible for binding to a synthetic receptor is suitable for targeting the synthetic receptor expressing cell to the cancer cell.

Suitable antigens include, but are not limited to, B-cell maturation antigen (BCMA), mesothelin (MSLN), prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD70, CD74, CD123, CD133, CD138, CD33, IL3Ra2, CS1, C-Met, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor (EGFR), Epidermal Growth Factor Receptor vIII (EGFRvIII), ERB3, ERB4, human telomerase reverse transcriptase (hTERT), Interleukin-13 receptor subunit alpha-2 (IL-13Rα2), x-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF- R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Rα, Latent Membrane Protein 1 (LMP1), MAG3, Neural cell-adhesion molecule (N-CAM/CD56), NY-ESO-1, and Trail Receptor (TRAIL R). It is understood that these or other cancer antigens can be utilized for targeting by a synthetic receptor disclosed herein.

In some embodiments, fusion protein disclosed herein is conjugated to a synthetic receptor targeting a tumor antigen selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1. In some embodiments, synthetic receptors provided herein include a target-binding domain that binds CD19. In some embodiments, synthetic receptors provided herein include a target-binding domain that binds CD20. In some embodiments, synthetic receptors provided herein include a target-binding domain that binds CD22. In some embodiments, synthetic receptors provided herein include a target-binding domain that binds CD30. In some embodiments, synthetic receptors provided herein include a target-binding domain that binds CD123. In some embodiments, synthetic receptors provided herein include a target-binding domain that binds CD138. In some embodiments, synthetic receptors provided herein include a target-binding domain that binds CD33. In some embodiments, synthetic receptors provided herein include a target-binding domain that binds CD70. In some embodiments, synthetic receptors provided herein include a target-binding domain that binds BCMA. In some embodiments, synthetic receptors provided herein include a target-binding domain that binds CS1. In some embodiments, synthetic receptors provided herein include a target-binding domain that binds C-Met. In some embodiments, synthetic receptors provided herein include a target-binding domain that binds IL13Ra2. In some embodiments, synthetic receptors provided herein include a target-binding domain that binds EGFRvIII. In some embodiments, synthetic receptors provided herein include a target-binding domain that binds CEA. In some embodiments, synthetic receptors provided herein include a target-binding domain that binds Her2. In some embodiments, synthetic receptors provided herein include a target-binding domain that binds GD2. In some embodiments, synthetic receptors provided herein include a target-binding domain that binds MAGE. In some embodiments, synthetic receptors provided herein include a target-binding domain that binds GPC3. In some embodiments, synthetic receptors provided herein include a target-binding domain that binds Mesothelin. In some embodiments, synthetic receptors provided herein include a target-binding domain that binds PSMA. In some embodiments, synthetic receptors provided herein include a target-binding domain that binds ROR1. In some embodiments, synthetic receptors provided herein include a target-binding domain that binds EGFR. In some embodiments, synthetic receptors provided herein include a target-binding domain that binds MUC1. In some embodiments, synthetic receptors provided herein include a target-binding domain that binds NY-ESO-1.

### 6.3.2 CARs

In some embodiments, the synthetic receptor is a CAR, and the fusion proteins provided herein can be co-expressed with a CAR in a cell. In some embodiments, a fusion protein provided herein can be conjugated to a CAR. CARs are synthetic receptors that retarget immune cells (e.g. T cells) to tumor surface antigens (Sadelain et al., Nat. Rev. Cancer. 3(1):35-45 (2003); Sadelain et al., Cancer Discovery 3(4):388-398 (2013)). CARs are engineered receptors that provide both antigen binding and immune cell activation functions. CARs can be used to graft the specificity of an antibody, such as a monoclonal antibody, onto an immune cell such as a T cell, a NK cell, or a macrophage. First-generation receptors link an antibody-derived tumor-binding element, such as an scFv, that is responsible for antigen recognition to either CD3zeta or Fc receptor signaling domains, which trigger T-cell activation. The advent of second-generation CARs, which combine activating and costimulatory signaling domains, has led to encouraging results in patients with chemorefractory B-cell malignancies (Brentjens et al., Science Translational Medicine 5(177):177ra38 (2013); Brentjens et al., Blood 118(18):4817-4828 (2011); Davila et al., Science Translational Medicine 6(224):224ra25 (2014); Grupp et al., N. Engl. J. Med. 368(16):1509-1518 (2013); Kalos et al., Science Translational Medicine 3(95):95ra73 (2011)). The extracellular antigen-binding domain of a CAR is usually derived from a monoclonal antibody (mAb) or from receptors or their ligands. Antigen binding by the CARs triggers phosphorylation of immunoreceptor tyrosine-based activation motifs (ITAMs) in the intracellular domain, initiating a signaling cascade required for cytolysis induction, cytokine secretion, and proliferation.

In some embodiments, a fusion protein provided herein can be conjugated to CAR that has an antigen binding domain that binds to a cancer antigen. In some embodiments, the CAR can be a “first generation,” “second generation” or “third generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. 21(2):215-223 (2009); Hollyman et al., J. Immunother. 32:169-180 (2009)).

“First generation” CARs are typically composed of an extracellular antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3η-chain, which is the primary transmitter of signals from endogenous T cell receptors (TCRs). “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second-generation” CARs comprises a cancer antigen-binding domain fused to an intracellular signaling domain capable of activating immune cells such as T cells and a co-stimulatory domain designed to augment immune cell, such as T cell, potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell. “Second generation” CARs provide both co-stimulation, for example, by CD28 or 4-1BB domains, and activation, for example, by a CD3ζ signaling domain. Studies have indicated that “Second Generation” CARs can improve the anti-tumor activity of T cells. “Third generation” CARs provide multiple co-stimulation, for example, by comprising both CD28 and 4-1BB domains, and activation, for example, by comprising a CD3ζ activation domain.

As described above, a CAR also contains a signaling domain that functions in the immune cell expressing the CAR. Such a signaling domain can be, for example, derived from CDC or Fc receptor γ (see Sadelain et al., Cancer Discov. 3:388-398 (2013)). In general, the signaling domain will induce persistence, trafficking and/or effector functions in the transduced immune cells such as T cells (Sharpe et al., Dis. Model Mech. 8:337-350 (2015); Finney et al., J. Immunol. 161:2791-2797 (1998); Krause et al., J. Exp. Med. 188:619-626 (1998)). In the case of CDC or Fc receptor γ, the signaling domain corresponds to the intracellular domain of the respective polypeptides, or a fragment of the intracellular domain that is sufficient for signaling. Exemplary signaling domains are described below in more detail.

CD3ζ. In a non-limiting embodiment, a CAR can comprise a signaling domain derived from a CD3ζ polypeptide, for example, a signaling domain derived from the intracellular domain of CD3ζ, which can activate or stimulate an immune cell, for example, a T cell. CD3ζ comprises 3 Immune-receptor-Tyrosine-based-Activation-Motifs (ITAMs), and transmits an activation signal to the cell, for example, a cell of the lymphoid lineage such as a T cell, after antigen is bound. A CD3ζ polypeptide can have an amino acid sequence corresponding to the sequence having GenBank No. NP_932170 (NP_932170.1, GI:37595565; see below), or fragments thereof. In one embodiment, the CD3ζ polypeptide has an amino acid sequence of amino acids 52 to 164 of the CD3ζ polypeptide sequence provided below, or a fragment thereof that is sufficient for signaling activity. An exemplary CAR has an intracellular domain comprising a CD3ζ polypeptide comprising amino acids 52 to 164 of the CD3ζ polypeptide sequence provided below. Another exemplary CAR has an intracellular domain comprising a CD3ζ polypeptide comprising amino acids 52 to 164 of the CD3ζ polypeptide provided below. Still another exemplary CAR has an intracellular domain comprising a CD3ζ polypeptide comprising amino acids 52 to 164 of the CD3ζ polypeptide provided below. See GenBank NP_932170 for reference to domains within CD3ζ, for example, signal peptide, amino acids 1 to 21; extracellular domain, amino acids 22 to 30; transmembrane domain, amino acids 31 to 51; intracellular domain, amino acids 52 to 164.

```
(NP_932170; SEQ ID NO: 92)
    1 MKWKALFTAA ILQAQLPITE AQSFGLLDPK
        LCYLLDGILF IYGVILTALF LRVKFSRSAD
  61 APAYQQGQNQ LYNELNLGRR EEYDVLDKRR
        GRDPEMGGKP QRRKNPQEGL YNELQKDKMA
121 EAYSEIGMKG ERRRGKGHDG LYQGLSTATK
        DTYDALHMQA LPPR
```

It is understood that a “CD3ζ nucleic acid” refers to a polynucleotide encoding a CD3ζ polypeptide. In one embodiment, the CD3ζ nucleic acid encoding the CD3ζ polypeptide comprised in the intracellular domain of a CAR, including exemplary CARs Mz, M28z, or MBBz, comprises a nucleotide sequence as set forth below.

(SEQ ID NO: 93)
AGAGTGAAGTTCAGCAGGAGCGCAGACGCCCCCGCGTACCAGCAGG
GCCAGAACCAGCTCTATAACGAGCTCAATCTAGGACGAAGAGAGG
AGTACGATGTTTTGGACAAGAGACGTGGCCGGGACCCTGAGATGG
GGGGAAAGCCGAGAAGGAAGAACCCTCAGGAAGGCCTGTACAATG
AACTGCAGAAAGATAAGATGGCGGAGGCCTACAGTGAGATTGGGA
TGAAAGGCGAGCGCCGGAGGGGCAAGGGGCACGATGGCCTTTACC
AGGGTCTCAGTACAGCCACCAAGGACACCTACGACGCCCTTCACA
TGCAGGCCCTGCCCCCTCGCTAA

In certain non-limiting embodiments, an intracellular domain of a CAR can further comprise at least one co-stimulatory signaling domain. In some embodiments, an intracellular domain of a CAR can comprise two co-stimulatory signaling domains. Such a co-stimulatory signaling domain can provide increased activation of an immune cell. A co-stimulatory signaling domain can be derived from a CD28 polypeptide, a 4-1BB polypeptide, an OX40 polypeptide, an ICOS polypeptide, a DAP10 polypeptide, a 2B4 polypeptide, a CD27 polypeptide, a CD30 polypeptide, a CD40 polypeptide and the like. CARs comprising an intracellular domain that comprises a co-stimulatory signaling region comprising 4-1BB, ICOS or DAP-10 have been described previously (see U.S. Pat. No. 7,446,190, which is incorporated herein by reference, which also describes representative sequences for 4-1BB, ICOS and DAP-10). In some embodiments, the intracellular domain of a CAR can comprise a co-stimulatory signaling region that comprises two co-stimulatory molecules, such as CD28 and 4-1BB (see Sadelain et al., Cancer Discov. 3(4):388-398 (2013)), or CD28 and OX40, or other combinations of co-stimulatory ligands, as disclosed herein.

CD28. Cluster of Differentiation 28 (CD28) is a protein expressed on T cells that provides co-stimulatory signals for T cell activation and survival. CD28 is the receptor for CD80 (B7.1) and CD86 (B7.2) proteins. In one embodiment, a CAR can comprise a co-stimulatory signaling domain derived from CD28. For example, as disclosed herein, a CAR can include at least a portion of an intracellular/cytoplasmic domain of CD28, for example an intracellular/cytoplasmic domain that can function as a co-stimulatory signaling domain. A CD28 polypeptide can have an amino acid sequence corresponding to the sequence having GenBank No. P10747 (P10747.1, GI:115973) or NP_006130 (NP_006130.1, GI:5453611), as provided below, or fragments thereof. If desired, CD28 sequences additional to the intracellular domain can be included in a CAR of the invention. For example, a CAR can comprise the transmembrane of a CD28 polypeptide. In one embodiment, a CAR can have an amino acid sequence comprising the intracellular domain of CD28 corresponding to amino acids 180 to 220 of CD28, or a fragment thereof. In another embodiment, a CAR can have an amino acid sequence comprising the transmembrane domain of CD28 corresponding to amino acids 153 to 179, or a fragment thereof. An exemplary CAR can comprise a co-stimulatory signaling domain corresponding to an intracellular domain of CD28. An exemplary CAR can also comprise a transmembrane domain derived from CD28. Thus, an exemplary CAR can comprise two domains from CD28, a co-stimulatory signaling domain and a transmembrane domain. In one embodiment, a CAR has an amino acid sequence comprising the transmembrane domain and the intracellular domain of CD28 and comprises amino acids 153 to 220 of CD28. In another embodiment, a CAR comprises amino acids 117 to 220 of CD28. Another exemplary CAR having a transmembrane domain and intracellular domain of CD28 is P28z. In one embodiment, a CAR can comprise a transmembrane domain derived from a CD28 polypeptide comprising amino acids 153 to 179 of the CD28 polypeptide provided below. See GenBank NP_006130 for reference to domains within CD28, for example, signal peptide, amino acids 1 to 18; extracellular domain, amino acids 19 to 152; transmembrane domain, amino acids 153 to 179; intracellular domain, amino acids 180 to 220. It is understood that sequences of CD28 that are shorter or longer than a specific delineated domain can be included in a CAR if desired.

```
(NP_006130; SEQ ID NO: 94)
    1 MLRLLLALNL FPSIQVTGNK ILVKQSPMLV
        AYDNAVNLSC KYSYNLFSRE FRASLHKGLD
  61 SAVEVCVVYG NYSQQLQVYS KTGENCDGKL
        GNESVTFYLQ NLYVNQTDIY FCKIEVMYPP
121 PYLDNEKSNG TIIHVKGKHL CPSPLFPGPS
        KPFWVLVVVG GVLACYSLLV TVAFIIFWVR
181 SKRSRLLHSD YMNMTPRRPG PTRKHYQPYA
        PPRDFAAYRS
```

It is understood that a “CD28 nucleic acid” refers to a polynucleotide encoding a CD28 polypeptide. In one embodiment, the CD28 nucleic acid encoding the CD28 polypeptide comprising the transmembrane domain and the intracellular domain, for example, the co-stimulatory signaling region, comprises a nucleotide sequence as set forth below.

(SEQ ID NO: 95)
ATTGAAGTTATGTATCCTCCTCCTTACCTAGACAATGAGAAGAGCAAT
GGAACCATTATCCATGTGAAAGGGAAACACCTTTGTCCAAGTCCCCTA
TTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTC
TGGGTGAGGAGTAAGAGGAGCAGGCTCCTGCACAGTGACTACATGAAC
ATGACTCCCCGCCGCCCCGGGCCCACCCGCAAGCATTACCAGCCCTAT
GCCCCACCACGCGACTTCGCAGCCTATCGCTCC

4-1BB. 4-1BB, also referred to as tumor necrosis factor receptor superfamily member 9, can act as a tumor necrosis factor (TNF) ligand and have stimulatory activity. In one embodiment, a CAR can comprise a co-stimulatory signaling domain derived from 4-1BB. A 4-1BB polypeptide can have an amino acid sequence corresponding to the sequence having GenBank No. P41273 (P41273.1, GI:728739) or NP_001552 (NP_001552.2, GI:5730095) or fragments thereof. In one embodiment, a CAR can have a co-stimulatory domain comprising the intracellular domain of 4-1BB corresponding to amino acids 214 to 255, or a fragment thereof. In another embodiment, a CAR can have a transmembrane domain of 4-1BB corresponding to amino acids 187 to 213, or a fragment thereof. An exemplary CAR is MBBz, which has an intracellular domain comprising a 4-1BB polypeptide (for example, amino acids 214 to 255 of NP_001552, SEQ ID NO:96). See GenBank NP_001552 for reference to domains within 4-1in, for example, signal peptide, amino acids 1 to 17; extracellular domain, amino acids 18 to 186; transmembrane domain, amino acids 187 to 213; intracellular domain, amino acids 214 to 255. It is understood that sequences of 4-1BB that are shorter or longer than a specific delineated domain can be included in a CAR, if desired. It is also understood that a “4-1BB nucleic acid” refers to a polynucleotide encoding a 4-1BB polypeptide.

| (NP_001552; SEQ ID NO: 96) | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

OX40. OX40, also referred to as tumor necrosis factor receptor superfamily member 4 precursor or CD134, is a member of the TNFR-superfamily of receptors. In one embodiment, a CAR can comprise a co-stimulatory signaling domain derived from OX40. An OX40 polypeptide can have an amino acid sequence corresponding to the sequence having GenBank No. P43489 (P43489.1, GI:1171933) or NP_003318 (NP_003318.1, GI:4507579), provided below, or fragments thereof. In one embodiment, a CAR can have a co-stimulatory domain comprising the intracellular domain of OX40 corresponding to amino acids 236 to 277, or a fragment thereof. In another embodiment, a CAR can have an amino acid sequence comprising the transmembrane domain of OX40 corresponding to amino acids 215 to 235 of OX40, or a fragment thereof. See GenBank NP_003318 for reference to domains within OX40, for example, signal peptide, amino acids 1 to 28; extracellular domain, amino acids 29 to 214; transmembrane domain, amino acids 215 to 235; intracellular domain, amino acids 236 to 277. It is understood that sequences of OX40 that are shorter or longer than a specific delineated domain can be included in a CAR, if desired. It is also understood that an “OX40 nucleic acid” refers to a polynucleotide encoding an OX40 polypeptide.

| (NP_003318; SEQ ID NO: 97) | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

ICOS. Inducible T-cell costimulator precursor (ICOS), also referred to as CD278, is a CD28-superfamily costimulatory molecule that is expressed on activated T cells. In one embodiment, a CAR can comprise a co-stimulatory signaling domain derived from ICOS. An ICOS polypeptide can have an amino acid sequence corresponding to the sequence having GenBank No. NP_036224 (NP_036224.1, GI:15029518), provided below, or fragments thereof. In one embodiment, a CAR can have a co-stimulatory domain comprising the intracellular domain of ICOS corresponding to amino acids 162 to 199 of ICOS. In another embodiment, a CAR can have an amino acid sequence comprising the transmembrane domain of ICOS corresponding to amino acids 141 to 161 of ICOS, or a fragment thereof. See GenBank NP_036224 for reference to domains within ICOS, for example, signal peptide, amino acids 1 to 20; extracellular domain, amino acids 21 to 140; transmembrane domain, amino acids 141 to 161; intracellular domain, amino acids 162 to 199. It is understood that sequences of ICOS that are shorter or longer than a specific delineated domain can be included in a CAR, if desired. It is also understood that an “ICOS nucleic acid” refers to a polynucleotide encoding an ICOS polypeptide.

| (NP_036224; SEQ ID NO: 98) | |
|---|---|
| 1 | MKSGLWYFFL FCLRIKVLTG EINGSANYEM FIFHNGGVQI LCKYPDIVQQ FKMQLLKGGQ |
| 61 | ILCDLTKTKG SGNTVSIKSL KFCHSQLSNN SVSFFLYNLD HSHANYYFCN LSIFDPPPFK |
| 121 | VTLTGGYLHI YESQLCCQLK FWLPIGCAAF VVVCILGCIL ICWLTKKKYS SSVHDPNGEY |
| 181 | MFMRAVNTAK KSRLTDVTL |

DAP10. DAP10, also referred to as hematopoietic cell signal transducer, is a signaling subunit that associates with a large family of receptors in hematopoietic cells. In one embodiment, a CAR can comprise a co-stimulatory domain derived from DAP10. A DAP10 polypeptide can have an amino acid sequence corresponding to the sequence having GenBank No. NP_055081.1 (GI:15826850), provided below, or fragments thereof. In one embodiment, a CAR can have a co-stimulatory domain comprising the intracellular domain of DAP10 corresponding to amino acids 70 to 93, or a fragment thereof. In another embodiment, a CAR can have a transmembrane domain of DAP10 corresponding to amino acids 49 to 69, or a fragment thereof. See GenBank NP_055081.1 for reference to domains within DAP10, for example, signal peptide, amino acids 1 to 19; extracellular domain, amino acids 20 to 48; transmembrane domain, amino acids 49 to 69; intracellular domain, amino acids 70 to 93. It is understood that sequences of DAP10 that are shorter or longer than a specific delineated domain can be included in a CAR, if desired. It is also understood that a “DAP10 nucleic acid” refers to a polynucleotide encoding a DAP10 polypeptide.

| (SEQ ID NO: 99) | |
|---|---|
| 1 | MIHLGHILFL LLLPVAAAQT TPGERSSLPA FYPGTSGSCS GCGSLSLPLL AGLVAADAVA |
| 61 | SLLIVGAVFL CARPRRSPAQ EDGKVYINMP GRG |

CD27: CD27 (TNFRSF7) is a transmembrane receptor expressed on subsets of human CD8+ and CD4+ T-cells, NKT cells, NK cell subsets and hematopoietic progenitors and induced in FOXP3+CD4 T-cells and B cell subsets. Previously studies have found that CD27 can either actively provide costimulatory signals that improve human T-cell survival and anti-tumor activity in vivo. (See Song and Powell; Oncoimmunology 1, no. 4 (2012): 547-549) In one embodiment, a CAR can comprise a co-stimulatory domain derived from CD27. A CD27 polypeptide can have an amino acid sequence corresponding to the sequence having UniProtKB/Swiss-Prot No.: P26842.2 (GI: 269849546), provided below, or fragments thereof.

| (SEQ ID NO: 100) | |
|---|---|
| 1 | MARPHPWWLC VLGTLVGLSA TPAPKSCPER HYWAQGKLCC QMCEPGTFLV KDCDQHRKAA |
| 61 | QCDPCIPGVS FSPDHHTRPH CESCRHCNSG LLVRNCTITA NAECACRNGW QCRDKECTEC |
| 121 | DPLPNPSLTA RSSQALSPHP QPTHLPYVSE MLEARTAGHM QTLADFRQLP ARTLSTHWPP |
| 181 | QRSLCSSDFI RILVIFSGMF LVFTLAGALF LHQRRKYRSN KGESPVEPAE PCHYSCPREE |
| 241 | EGSTIPIQED YRKPEPACSP |

In one embodiment, a CAR can have a co-stimulatory domain comprising the intracellular domain of CD27 or a fragment thereof. In another embodiment, a CAR can have a transmembrane domain of CD27 or a fragment thereof. It is understood that sequences of CD27 that are shorter or longer than a specific delineated domain can be included in a CAR, if desired. It is also understood that a “CD27 nucleic acid” refers to a polynucleotide encoding an CD27 polypeptide.

CD30: CD30 and its ligand (CD30L) are members of the tumor necrosis factor receptor (TNFR) and tumor necrosis factor (TNF) superfamilies, respectively. CD30, in many respects, behaves similarly to Ox40 and enhances proliferation and cytokine production induced by TCR stimulation. (Goronzy and Weyand, Arthritis research & therapy 10, no. S1 (2008): S3.) In one embodiment, a CAR can comprise a co-stimulatory domain derived from CD30. A CD30 polypeptide can have an amino acid sequence corresponding to the sequence having GenBank No.: AAA51947.1 (GI: 180096), provided below, or fragments thereof.

| (SEQ ID NO: 101) | |
|---|---|
| 1 | MRVLLAALGL LFLGALRAFP QDRPFEDTCH GNPSHYYDKA VRRCCYRCPM GLFPTQQCPQ |
| 61 | RPTDCRKQCE PDYYLDEADR CTACVTCSRD DLVEKTPCAW NSSRVCECRP GMFCSTSAVN |
| 121 | SCARCFFHSV CPAGMIVKFP GTAQKNTVCE PASPGVSPAC ASPENCKEPS SGTIPQAKPT |
| 181 | PVSPATSSAS TMPVRGGTRL AQEAASKLTR APDSPSSVGR PSSDPGLSPT QPCPEGSGDC |
| 241 | RKQCEPDYYL DEAGRCTACV SCSRDDLVEK TPCAWNSSRT CECRPGMICA TSATNSCARC |
| 301 | VPYPICAAET VTKPQDMAEK DTTFEAPPLG TQPDCNPTPE NGEAPASTSP TQSLLVDSQA |
| 361 | SKTLPIPTSA PVALSSTGKP VLDAGPVLFW VILVLVVVVG SSAFLLCHRR ACRKRIRQKL |
| 421 | HLCYPVQTSQ PKLELVDSRP RRSSTQLRSG ASVTEPVAEE RGLMSQPLME TCHSVGAAYL |
| 481 | ESLPLQDASP AGGPSSPRDL PEPRVSTEHT NNKIEKIYIM KADTVIVGTV KAELPEGRGL |
| 541 | AGPAEPELEE ELEADHTPHY PEQETEPPLG SCSDVMLSVE EEGKEDPLPT AASGK |

In one embodiment, a CAR can have a co-stimulatory domain comprising the intracellular domain of CD30 or a fragment thereof. In another embodiment, a CAR can have a transmembrane domain of CD30 or a fragment thereof. It is understood that sequences of CD30 that are shorter or longer than a specific delineated domain can be included in a CAR, if desired. It is also understood that a “CD30 nucleic acid” refers to a polynucleotide encoding an CD30 polypeptide.

CD40: CD40 and its ligand, CD40L or CD154, were first identified as instrumental in T-cell-dependent B-cell activation. The pathway is now recognized as a mechanism to activate APCs and to enhance their potential to activate T cells. CD154-mediated CD40 stimulation provides an important feedback mechanism for the initial co-stimulatory pathway of CD28-CD80/CD86. (Goronzy and Weyand, Arthritis research & therapy 10, no. S1 (2008): S3.) In one embodiment, a CAR can comprise a co-stimulatory domain derived from CD40. A CD40 polypeptide can have an amino acid sequence corresponding to the sequence having UniProtKB/Swiss-Prot No.: P25942.1 GI: 269849546 provided below, or fragments thereof.

| (SEQ ID NO: 102) | |
|---|---|
| 1 | MVRLPLQCVL WGCLLTAVHP EPPTACREKQ YLINSQCCSL CQPGQKLVSD CTEFTETECL |
| 61 | PCGESEFLDT WNRETHCHQH KYCDPNLGLR VQQKGTSETD TICTCEEGWH CTSEACESCV |
| 121 | LHRSCSPGFG VKQIATGVSD TICEPCPVGF FSNVSSAFEK CHPWTSCETK DLVVQQAGTN |
| 181 | KTDVVCGPQD RLRALVVIPI IFGILFAILL VLVFIKKVAK KPTNKAPHPK QEPQEINFPD |
| 241 | DLPGSNTAAP VQETLHGCQP VTQEDGKESR ISVQERQ |

In one embodiment, a CAR can have a co-stimulatory domain comprising the intracellular domain of CD40 or a fragment thereof. In another embodiment, a CAR can have a transmembrane domain of CD40 or a fragment thereof. It is understood that sequences of CD40 that are shorter or longer than a specific delineated domain can be included in a CAR, if desired. It is also understood that a “CD40 nucleic acid” refers to a polynucleotide encoding an CD40 polypeptide.

The extracellular domain of a CAR can be fused to a leader or a signal peptide that directs the nascent protein into the endoplasmic reticulum and subsequent translocation to the cell surface. It is understood that, once a polypeptide containing a signal peptide is expressed at the cell surface, the signal peptide has generally been proteolytically removed during processing of the polypeptide in the endoplasmic reticulum and translocation to the cell surface. Thus, a polypeptide such as a CAR is generally expressed at the cell surface as a mature protein lacking the signal peptide, whereas the precursor form of the polypeptide includes the signal peptide. A signal peptide or leader can be essential if a CAR is to be glycosylated and/or anchored in the cell membrane. The signal sequence or leader is a peptide sequence generally present at the N-terminus of newly synthesized proteins that directs their entry into the secretory pathway. The signal peptide is covalently joined to the N-terminus of the extracellular antigen-binding domain of a CAR as a fusion protein. In one embodiment, the signal peptide comprises a CD8 polypeptide comprising amino acids MALPVTALLLPLALLLHAARP (SEQ ID NO:116). It is understood that use of a CD8 signal peptide is exemplary. Any suitable signal peptide, as are well known in the art, can be applied to a CAR to provide cell surface expression in an immune cell (see Gierasch Biochem. 28:923-930 (1989); von Heijne, J. Mol. Biol. 184 (1):99-105 (1985)). Particularly useful signal peptides can be derived from cell surface proteins naturally expressed in the immune cell provided herein, including any of the signal peptides of the polypeptides disclosed herein. Thus, any suitable signal peptide can be utilized to direct a CAR to be expressed at the cell surface of an immune cell provided herein.

In certain non-limiting embodiments, an extracellular antigen-binding domain of a CAR can comprise a linker sequence or peptide linker connecting the heavy chain variable region and light chain variable region of the extracellular antigen-binding domain. In one non-limiting example, the linker comprises amino acids having the sequence set forth in GGGGSGGGGSGGGGS (SEQ ID NO:1).

In certain non-limiting embodiments, a CAR can also comprise a spacer region or sequence that links the domains of the CAR to each other. For example, a spacer can be included between a signal peptide and an antigen binding domain, between the antigen binding domain and the transmembrane domain, between the transmembrane domain and the intracellular domain, and/or between domains within the intracellular domain, for example, between a stimulatory domain and a co-stimulatory domain. The spacer region can be flexible enough to allow interactions of various domains with other polypeptides, for example, to allow the antigen binding domain to have flexibility in orientation in order to facilitate antigen recognition. The spacer region can be, for example, the hinge region from an IgG, the CH₂CH₃ (constant) region of an immunoglobulin, and/or portions of CD3 (cluster of differentiation 3) or some other sequence suitable as a spacer.

The transmembrane domain of a CAR generally comprises a hydrophobic alpha helix that spans at least a portion of the membrane. Different transmembrane domains result in different receptor stability. After antigen recognition, receptors cluster and a signal is transmitted to the cell. In an embodiment, the transmembrane domain of a CAR can be derived from another polypeptide that is naturally expressed in the immune cell. In one embodiment, a CAR can have a transmembrane domain derived from CD8, CD28, CD3ζ, CD4, 4-1BB, OX40, ICOS, CTLA-4, PD-1, LAG-3, 2B4, BTLA, or other polypeptides expressed in the immune cell. Optionally, the transmembrane domain can be derived from a polypeptide that is not naturally expressed in the immune cell, so long as the transmembrane domain can function in transducing signal from antigen bound to the CAR to the intracellular signaling and/or co-stimulatory domains. It is understood that the portion of the polypeptide that comprises a transmembrane domain of the polypeptide can include additional sequences from the polypeptide, for example, additional sequences adjacent on the N-terminal or C-terminal end of the transmembrane domain, or other regions of the polypeptide, as desired.

CD8. Cluster of differentiation 8 (CD8) is a transmembrane glycoprotein that serves as a co-receptor for the T cell receptor (TCR). CD8 binds to a major histocompatibility complex (MHC) molecule and is specific for the class I NMC protein. In one embodiment, a CAR can comprise a transmembrane domain derived from CD8. A CD8 polypeptide can have an amino acid sequence corresponding to the sequence having GenBank No. NP_001139345.1 (GI:225007536), as provided below, or fragments thereof. In one embodiment, a CAR can have an amino acid sequence comprising the transmembrane domain of CD8 corresponding to amino acids 183 to 203, or fragments thereof. In one embodiment, an exemplary CAR has a transmembrane domain derived from a CD8 polypeptide. In one non-limiting embodiment, a CAR can comprise a transmembrane domain derived from a CD8 polypeptide comprising amino acids 183 to 203. In addition, a CAR can comprise a hinge domain comprising amino acids 137-182 of the CD8 polypeptide provided below. In another embodiment, a CAR can comprise amino acids 137-203 of the CD8 polypeptide provided below. In yet another embodiment, a CAR can comprise amino acids 137 to 209 of the CD8 polypeptide provided below. See GenBank NP_001139345.1 for reference to domains within CD8, for example, signal peptide, amino acids 1 to 21; extracellular domain, amino acids 22 to 182; transmembrane domain amino acids, 183 to 203; intracellular domain, amino acids 204 to 235. It is understood that additional sequence of CD8 beyond the transmembrane domain of amino acids 183 to 203 can be included in a CAR, if desired. It is further understood that sequences of CD8 that are shorter or longer than a specific delineated domain can be included in a CAR, if desired. It also is understood that a “CD8 nucleic acid” refers to a polynucleotide encoding a CD8 polypeptide.

| (NP_001139345.1; SEQ ID NO: 103) | |
|---|---|
| 1 | MALPVTALLL PLALLLHAAR PSQFRVSPLD RTWNLGETVE LKCQVLLSNP TSGCSWLFQP |
| 61 | RGAAASPTFL LYLSQNKPKA AEGLDTQRFS GKRLGDTFVL TLSDFRRENE GYYFCSALSN |
| 121 | SIMYFSHFVP VFLPAKPTTT PAPRPPTPAP TIASQPLSLR PEACRPAAGG AVHTRGLDFA |
| 181 | CDIYIWAPLA GTCGVLLLSL VITLYCNHRN RRRVCKCPRP VVKSGDKPSL SARYV |

CD4. Cluster of differentiation 4 (CD4), also referred to as T-cell surface glycoprotein CD4, is a glycoprotein found on the surface of immune cells such as T helper cells, monocytes, macrophages, and dendritic cells. In one embodiment, a CAR can comprise a transmembrane domain derived from CD4. CD4 exists in various isoforms. It is understood that any isoform can be selected to achieve a desired function. Exemplary isoforms include isoform 1 (NP_000607.1, GI:10835167), isoform 2 (NP_001181943.1, GI:303522479), isoform 3 (NP_001181944.1, GI:303522485; or NP_001181945.1, GI:303522491; or NP_001181946.1, GI:303522569), and the like. One exemplary isoform sequence, isoform 1, is provided below. In one embodiment, a CAR can have an amino acid sequence comprising the transmembrane domain of CD4 corresponding to amino acids 397 to 418, or fragments thereof. See GenBank NP_000607.1 for reference to domains within CD4, for example, signal peptide, amino acids 1 to 25; extracellular domain, amino acids 26 to 396; transmembrane domain amino acids, 397 to 418; intracellular domain, amino acids 419 to 458. It is understood that additional sequence of CD4 beyond the transmembrane domain of amino acids 397 to 418 can be included in a CAR, if desired. It is further understood that sequences of CD4 that are shorter or longer than a specific delineated domain can be included in a CAR, if desired. It also is understood that a “CD4 nucleic acid” refers to a polynucleotide encoding a CD4 polypeptide.

| (NP_000607.1; SEQ ID NO: 104) | |
|---|---|
| 1 | MNRGVPFRHL LLVLQLALLP AATQGKKVVL GKKGDTVELT CTASQKKSIQ FHWKNSNQIK |
| 61 | ILGNQGSFLT KGPSKLNDRA DSRRSLWDQG NFPLIIKNLK IEDSDTYICE VEDQKEEVQL |
| 121 | LVFGLTANSD THLLQGQSLT LTLESPPGSS PSVQCRSPRG KNIQGGKTLS VSQLELQDSG |
| 181 | TWTCTVLQNQ KKVEFKIDIV VLAFQKASSI VYKKEGEQVE FSFPLAFTVE KLTGSGELWW |
| 241 | QAERASSSKS WITFDLKNKE VSVKRVTQDP KLQMGKKLPL HLTLPQALPQ YAGSGNLTLA |
| 301 | LEAKTGKLHQ EVNLVVMRAT QLQKNLTCEV WGPTSPKLML SLKLENKEAK VSKREKAVWV |
| 361 | LNPEAGMWQC LLSDSGQVLL ESNIKVLPTW STPVQPMALI VLGGVAGLLL FIGLGIFFCV |
| 421 | RCRHRRRQAE RMSQIKRLLS EKKTCQCPHR FQKTCSPI |

In addition to cytotoxic T cells, regulatory T-cells (Tregs) are found to be efficiently engineered with a predetermined antigen-specificity via transfection of viral vectors encoding specific CAR (CAR-Tregs). CAR-Tregs engineered in a non-MHC restricted manner have the advantage of widespread applications, especially in transplantation and autoimmunity (Zhang et al. (2018) Front. Immunol. 9:2359).

In addition to T cells, CAR can be engineered into other types of immune cells, such as NK cells, macrophages, or B cells. In some embodiments, the engineered cell is a NK cell. In some embodiments, the synthetic receptor is a CAR that retargets NK cells to tumor surface antigens (see e.g., Hu et al. Acta Pharmacol Sin 39, 167-176 (2018)). CAR-NK cells can use the first generation of CAR constructs that contain CD3ζ as an intracellular signaling domain or the second generation of CAR constructs that express a second signaling domain (e.g., CD28, 4-1BB) in conjunction with CD3ζ. In general, the second generation of CARs in NK cells is more active than first-generation CARs. In some embodiments, CAR constructs are based on the activating features of NK cells. For example, DNAX-activation protein 12 (DAP12) is known to activate signaling for NK cells.

DAP12. DAP12 is found in cells of the myeloid lineage, such as macrophages and granulocytes, where it associates, for instance, with the triggering receptor expressed on myeloid cell members (TREM) and MDL1 (myeloid DAP12-associating lectin 1/CLEC5A), both involved in inflammatory responses against pathogens like viruses and bacteria. In the lymphoid lineage, DAP12 is expressed in NK cells and associates with activating receptors such as the C-type lectin receptor NKG2C, the natural cytotoxicity receptor NKp44, and the short-tailed KIR3DS1 and KIR2DS1/2/5, respectively. In particular, NGK2C is the dominant activating NK cell receptor for controlling CMV infection in both humans and mice. It was found that a DAP12-containing CAR generated sufficient activating signals in NK cells upon cross-linking with its Ag. Topfer et al., J Immunol 194:3201-12 (2015). In one embodiment, a CAR can comprise a co-stimulatory domain derived from DAP12. A DAP12 polypeptide can have an amino acid sequence corresponding to the sequence having GenBank No. AAD09437.1 (GI: 2905996), provided below, or fragments thereof. In one embodiment, a CAR can have a signaling domain comprising the intracellular domain of DAP12, or a fragment thereof. In another embodiment, a CAR can have a transmembrane domain of DAP12, or a fragment thereof. It is understood that a “DAP12 nucleic acid” refers to a polynucleotide encoding a DAP12 polypeptide.

| (SEQ ID NO: 118) | |
|---|---|
| 1 | MGGLEPCSRL LLLPLLLAVS GLRPVQAQAQ SDCSCSTVSP GVLAGIVMGD LVLTVLIALA |
| 61 | VYFLGRLVPR GRGAAEAATR KQRITETESP YQELQGQRSD VYSDLNTQRP YYK |

As the receptor NKG2D is important for NK cell activity and the NKG2D ligands are commonly overexpressed on various tumors, a unique CAR construct that contains NKG2D as the ectodomain and links signaling proteins such as DAP10 and CD3ζ has been developed and can be co-expressed or conjugated with the fusion proteins disclosed herein in an engineered cell. These CAR-expressing NK cells have enhanced cytotoxicity against a wide spectrum of tumor subtypes, with the best responses observed in ALL, osteosarcoma, prostate carcinoma, and rhabdomyosarcoma (Chang et al., Cancer Res 2013; 73: 1777-86). A human NKG2D polypeptide can have an amino acid sequence corresponding to the sequence having GenBank No. CAA04925.1 (GI: 2980865), provided below, or fragments thereof. It is understood that a “NKG2D nucleic acid” refers to a polynucleotide encoding a NKG2D polypeptide.

| (SEQ ID NO: 119) | |
|---|---|
| 1 | MGWIRGRRSR HSWEMSEFHN YNLDLKKSDF STRWQKQRCP VVKSKCRENA SPFFFCCFIA |
| 61 | VAMGIRFIIM VAIWSAVFLN SLFNQEVQIP LTESYCGPCP KNWICYKNNC YQFFDESKNW |
| 121 | YESQASCMSQ NASLLKVYSK EDQDLLKLVK SYHWMGLVHI PTNGSWQWED GSILSPNLLT |
| 181 | IIEMQKGDCA LYASSFKGYI ENCSTPNTYI CMQRTV |

In some embodiments, CAR can be engineered into macrophages. In some embodiments, the engineered cell is a macrophage. For example, a family of Chimeric Antigen Receptors for Phagocytosis (CAR-Ps) that directed macrophages to engulf specific targets, including cancer cells has been generated (Morrissey et al., eLife 2018; 7:e36688). Specifically, the cytosolic domains from MEGF10 and FcRV can trigger engulfment independently of their native extracellular domain and CAR-Ps can drive specific engulfment of antigen-coated synthetic particles and whole human cancer cells. Addition of a tandem PI3K recruitment domain can further increase cancer cell engulfment (Morrissey et al., eLife 2018; 7:e36688).

MEGF10 Multiple epidermal growth factor-like domains protein 10 (MEGF10), also known as EMARDD or SR-F3, is a membrane receptor involved in phagocytosis by macrophages and astrocytes of apoptotic cells. The intracellular domain of Megf10 has cytosolic Immunore-ceptor Tyrosine-based Activation Motifs (ITAMs) that are phosphorylated by Src family kinases. In one embodiment, a CAR can comprise a transmembrane domain derived from MEGF10. In one embodiment, a CAR can comprise a co-stimulatory domain derived from MEGF10. A MEGF10 polypeptide can have an amino acid sequence corresponding to the sequence having GenBank No. AAH20198.1 (GI: 18044366), provided below, or fragments thereof. In one embodiment, a CAR can have a co-stimulatory domain comprising the intracellular domain of MEGF10, or a fragment thereof. In another embodiment, a CAR can have a transmembrane domain of MEGF10, or a fragment thereof. It is understood that a “MEGF 10 nucleic acid” refers to a polynucleotide encoding a MEGF10 polypeptide.

| (SEQ ID NO: 120) | |
|---|---|
| 1 | MVISLNSCLS FICLLLCHWI GTASPLNLED PNVCSHWESY SVTVQESYPH PFDQIYYTSC |
| 61 | TDILNWFKCT RHRVSYRTAY RHGEKTMYRR KSQCCPGFYE SGEMCVPHCA DKCVHGRCIA |
| 121 | PNTCQCEPGW GGTNCSSACD GDHWGPHCTS RCQCKNGALC NPITGACHCA AGFRGWRCED |
| 181 | RCEQGTYGND CHQRCQCQNG ATCDHVTGEC RCPPGYTGAF CEDLCPPGKH GPQCEQRCPC |
| 241 | QNGGVCHHVT GECSCPSGWM GTVCGQPCPE GRFGKNCSQE CQCHNGGTCD AATGQCHCSP |
| 301 | GYTGERCQDE CPVGTYGVLC AETCQCVNGG KCYHVSGACL CEAGFAGERC EARLCPEGLY |
| 361 | GIKCDKRCPC HLENTHSCHP MSGECACKPG WSGLYCNETC SPGFYGEACQ QICSCQNGAD |
| 421 | CDSVTGKCTC APGFKGIDCS TPCPLGTYGI NCSSRCGCKN DAVCSPVDGS CTCKAGWHGV |
| 481 | DCSIRCPSGT WGFGCNLTCQ CLNGGACNTL DGTCTCAPGW RGEKCELPCQ DGTYGLNCAE |
| 541 | RCDCSHADGC HPTTGHCRCL PGWSGLF |

FcRγ Activating types of IgG receptor FcγRs form multimeric complexes including the Fc receptor common 7 chain (FcRγ) that contains an intracellular tyrosine-based activating motif (ITAM), whose activation triggers oxidative bursts, cytokine release, phagocytosis, antibody-dependent cell-mediated cytotoxicity, and degranulation. In one embodiment, a CAR can comprise a transmembrane domain derived from FcRγ. In one embodiment, a CAR can comprise a co-stimulatory domain derived from FcRγ. An FcRγ polypeptide can have an amino acid sequence corresponding to the sequence having NCBI Reference Sequence: NP_004097.1 (GI: 4758344), provided below, or fragments thereof. In one embodiment, a CAR can have a co-stimulatory domain comprising the intracellular domain of FcRγ, or a fragment thereof. In another embodiment, a CAR can have a transmembrane domain of FcRγ, or a fragment thereof. It is understood that an “FcRγ nucleic acid” refers to a polynucleotide encoding an FcRγ polypeptide.

| (SEQ ID NO: 121) | |
|---|---|
| 1 | MIPAVVLLLL LLVEQAAALG EPQLCYILDA ILFLYGIVLT LLYCRLKIQV RKAAITSYEK |
| 61 | SDGVYTGLST RNQETYETLK HEKPPQ |

CARs provided herein can include a target-binding domain as disclosed above. In some embodiments, fusion proteins disclosed herein can be co-expressed with a CAR targeting a tumor antigen selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1 in a cell. In some embodiments, fusion proteins disclosed herein is conjugated to a CAR targeting a tumor antigen selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1. In some embodiments, fusion proteins provided herein can be co-expressed or conjugated with a CAR that targets CD19 (CAR19). In some embodiments, fusion proteins provided herein can be conjugated to a CAR. In some embodiments, fusion proteins provided herein can be conjugated to a CAR that binds CD19 (CAR19). In some embodiments, the amino acid sequence of CAR19 is SEQ ID NO:74, which can be encoded by, for example, the nucleotide sequence of SEQ ID NO:20. In some embodiments, the amino acid sequence of CAR19 is SEQ ID NO:75. In some embodiments, the amino acid sequence of CAR19 is SEQ ID NO:76. In some embodiments, synthetic receptors provided herein include a target-binding domain that binds BCMA. In some embodiments, the amino acid sequence of the CAR that binds BCMA is SEQ ID NO:77. In some embodiments, the amino acid sequence of the CAR that binds BCMA is SEQ ID NO:78. In some embodiments, the amino acid sequence of the CAR that binds BCMA is SEQ ID NO:79. In some embodiments, the amino acid sequence of the CAR that binds BCMA is SEQ ID NO:80. In some embodiments, the amino acid sequence of the CAR that binds BCMA is SEQ ID NO:136, which can be encoded by, for example, the nucleotide sequence of SEQ ID NO:137.

| CAR | Amino Acid Sequences |
|---|---|
| Anti-CD19 | MALPVTALLLPLALLLHAARPDIQMTQTTSSLSASLGDRVTISCRASQDISKYL |
| | NWYQQKPDGTVKLLIYHTSRLHSGVPSRFSGSGSGTDYSLTISNLEQEDIATYF |
| | CQQGNTLPYTFGGGTKLEITGGGGSGGGGSGGGGSEVKLQESGPGLVAPSQS |
| | LSVTCTVSGVSLPDYGVSWIRQPPRKGLEWLGVIWGSETTYYNSALKSRLTII |
| | KDNSKSQVFLKMNSLQTDDTAIYYCAKHYYYGGSYAMDYWGQGTSVTVSS |
| | TRTTTPAPRPPTPAPTIASQPLSLRPEACRPAAGGAVHTRGLDFACDIYIWAPL |
| | AGTCGVLLLSLVITLYCKRGRKKLLYIFKQPFMRPVQTTQEEDGCSCRFPEEE |
| | EGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLST |
| | ATKDTYDALHMQALPPR(SEQ ID NO: 74) |
| Anti-CD19 | MALPVTALLLPLALLLHAARPDIQMTQTTSSLSASLGDRVTISCRASQDISKYL |
| | NWYQQKPDGTVKLLIYHTSRLHSGVPSRFSGSGSGTDYSLTISNLEQEDIATYF |
| | CQQGNTLPYTFGGGTKLEITGGGGSGGGGSGGGGSEVKLQESGPGLVAPSQS |
| | LSVTCTVSGVSLPDYGVSWIRQPPRKGLEWLGVIWGSETTYYNSALKSRLTII |
| | KDNSKSQVFLKMNSLQTDDTAIYYCAKHYYYGGSYAMDYWGQGTSVTVSS |
| | TTTPAPRPPTPAPTIASQPLSLRPEACRPAAGGAVHTRGLDFACDIYIWAPLAG |
| | TCGVLLLSLVITLYCKRGRKKLLYIFKQPFMRPVQTTQEEDGCSCRFPEEEEG |
| | GCELRVKFSRSADAPAYKQGQNQLYNELNLGRREEYDVLDKRRGRDPEMGG |
| | KPRRKNPQEGLYNELQKDKMAEAYSEIGMKGERRRGKGHDGLYQGLSTATK |
| | DTYDALHMQALPPR (SEQ ID NO: 75) |
| Anti-CD19 | MALPVTALLLPLALLLHAARPDIQMTQTTSSLSASLGDRVTISCRASQDISKYL |
| | NWYQQKPDGTVKLLIYHTSRLHSGVPSRFSGSGSGTDYSLTISNLEQEDIATYF |
| | CQQGNTLPYTFGGGTKLEITGGGGSGGGGSGGGGSEVKLQESGPGLVAPSQS |
| | LSVTCTVSGVSLPDYGVSWIRQPPRKGLEWLGVIWGSETTYYNSALKSRLTII |
| | KDNSKSQVFLKMNSLQTDDTAIYYCAKHYYYGGSYAMDYWGQGTSVTVSS |
| | TTTPAPRPPTPAPTIASQPLSLRPEACRPAAGGAVHTRGLDFACDIYFWVLVV |
| | VGGVLACYSLLVTVAFIIFWVRSKRSRGGHSDYMNMTPRRPGPTRKHYQPYA |
| | PPRDFAAYRSVKFSRSADAPAYQQGQNQLYNELNLGRREEYDVLDKRRGRD |
| | PEMGGKPRRKNPQEGLYNELQKDKMAEAYSEIGMKGERRRGKGHDGLYQG |
| | LSTATKDTYDALHMQALPPR (SEQ ID NO: 76) |
| Anti-BCMA | MALPVTALLLPLALLLHAARPEVQLQASGGGLAQPGGSLRLSCAASGRTFST |
| | YFMAWFRQPPGKGLEYVGGIRWSDGVPHYADSVKGRFTISRDNAKNTVYLQ |
| | MNSLRAEDTAVYFCASRGIADGSDFGSYGQGTQVTVSSPAKPTTTPAPRPPTP |
| | APTIASQPLSLRPEACRPAAGGAVHTRGLDFACDIYIWAPLAGTCGVLLLSLVI |
| | TLYCKRGRKKLLYIFKQPFMRPVQTTQEEDGCCRFPEEEEGGCELRVKFSRS |
| | ADAPAYQQGQNQLYNELNLGRREEYDVLDKRRGRDPEMGGKPQRRKNPQE |
| | GLYNELQKDKMAEAYSEIGMKGERRRGKGHDGLYQGLSTATKDTYDALHM |
| | QALPPR (SEQ ID NO: 77) |
| Anti-BCMA | MALPVTALLLPLALLLHAARPQVKLEESGGGLVQAGRSLRLSCAASEHTFSSH |
| | VMGWFRQAPGKERESVAVIGWRDISTSYADSVKGRFTISRDNAKKTLYLQM |
| | NSLKPEDTAVYYCAARRIDAADFDSWGQGTQVTVSSGGGGSGGGGSGGGGS |
| | GGGGSGGGGSAVQLVESGGGLVQAGDSLRLTCTASGRAFSTYFMAWFRQAP |
| | GKEREFVAGIAWSGGSTAYADSVKGRFTISRDNAKNTVYLQMNSLKSEDTAV |
| | YYCASRGIEVEEFGAWGQGTQVTVSSTSTRTTTPAPRPPTPAPTIASQPLSLRP |
| | EACRPAAGGAVHTRGLDFACDIYIWAPLAGTCGVLLLSLVITLYCKRGRKKL |
| | LYIFKQPFMRPVQTTQEEDGCSCRFPEEEEGGCELRVKFSRSADAPAYQQGQN |
| | QLYNELNLGRREEYDVLDKRRGRDPEMGGKPRRKNPQEGLYNELQKDKMA |
| | EAYSEIGMKGERRRGKGHDGLYQGLSTATKDTYDALHMQALPPR |
| | (SEQ ID NO: 78) |
| Anti-BCMA | MALPVTALLLPLALLLHAARPQVKLEESGGGLVQAGRSLRLSCAASEHTFSSH |
| | VMGWFRQAPGKERESVAVIGWRDISTSYADSVKGRFTISRDNAKKTLYLQM |
| | NSLKPEDTAVYYCAARRIDAADFDSWGQGTQVTVSSGGGGSGGGGSAVQLV |
| | ESGGGLVQAGDSLRLTCTASGRAFSTYFMAWFRQAPGKEREFVAGIAWSGG |
| | STAYADSVKGRFTISRDNAKNTVYLQMNSLKSEDTAVYYCASRGIEVEEFGA |
| | WGQGTQVTVSSTSTRTTTPAPRPPTPAPTIASQPLSLRPEACRPAAGGAVHTR |
| | GLDFACDIYIWAPLAGTCGVLLLSLVITLYCKRGRKKLLYIFKQPFMRPVQTT |
| | QEEDGCSCRFPEEEEGGCELRVKFSRSADAPAYQQGQNQLYNELNLGRREEY |
| | DVLDKRRGRDPEMGGKPRRKNPQEGLYNELQKDKMAEAYSEIGMKGERRR |
| | GKGHDGLYQGLSTATKDTYDALHMQALPPR (SEQ ID NO: 79) |
| Anti-BCMA | MALPVTALLLPLALLLHAARPDIVLTQSPASLAVSLGERATINCRASESVSVIG |
| | AHLIHWYQQKPGQPPKLLIYLASNLETGVPARFSGSGSGTDFTLTISSLQAEDA |
| | AIYYCLQSRIFPRTFGQGTKLEIKGSTSGSGKPGSGEGSTKGQVQLVQSGSELK |
| | KPGASVKVSCKASGYTFTDYSINWVRQAPGQGLEWMGWINTETREPAYAYD |
| | FRGRFVFSLDTSVSTAYLQISSLKAEDTAVYYCARDYSYAMDYWGQGTLVT |
| | VSSAAATTTPAPRPPTPAPTIASQPLSLRPEACRPAAGGAVHTRGLDFACDIYI |
| | WAPLAGTCGVLLLSLVITLYCKRGRKKLLYIFKQPFMRPVQTTQEEDGCSCRF |
| | PEEEEGGCELRVKFSRSADAPAYQQGQNQLYNELNLGRREEYDVLDKRRGR |
| | DPEMGGKPRRKNPQEGLYNELQKDKMAEAYSEIGMKGERRRGKGHDGLYQ |
| | GLSTATKDTYDALHMQALPPR (SEQ ID NO: 80) |
| Anti-BCMA | MALPVTALLLPLALLLHAARPQVQLVESGGGLVQAGGSLRLSCAASGLTFSE |
| | YTMGWFRQPPGKEREFVAAISGGGIPYYRTTVKGRFSISRDNAQNTVALEMN |
| | SLKPEDTAVYYCAADARAVMTVTPNYWGQGTQVTVSSEPKTPKPQDTTTPA |
| | PRPPTPAPTIASQPLSLRPEACRPAAGGAVHTRGLDFACDIYIWAPLAGTCGVL |
| | LLSLVITLYCKRGRKKLLYIFKQPFMRPVQTTQEEDGCSCRFPEEEEGGCELRV |
| | KFSRSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTYDAL |
| | HMQALPPR (SEQ ID NO: 136) |

### 6.3.3 TCRs

Fusion proteins provided herein can be co-expressed with a synthetic receptor in a cell, or conjugated to a synthetic receptor. In some embodiments, the synthetic receptor comprises a TCR, and fusion proteins provided herein can be co-expressed with a TCR in a cell, or conjugated to a TCR. In some embodiments, the synthetic receptor can engage and activate endogenous TCR, and fusion proteins provided herein can be co-expressed or conjugated with a synthetic receptor that can engage and activate endogenous TCR.

T cell receptors (TCRs) are antigen-specific molecules that are responsible for recognizing antigenic peptides presented in the context of a product of the MHC on the surface of antigen presenting cells (APCs) or any nucleated cells. This system endows T cells, via their TCRs, with the potential ability to recognize the entire array of intracellular antigens expressed by a cell (including virus proteins) that are processed into short peptides, bound to an intracellular MHC molecule, and delivered to the surface as a peptide-MHC complex. This system allows foreign protein (e.g., mutated cancer antigen or virus protein) or aberrantly expressed protein to serve a target for T cells (e.g. Davis and Bjorkman (1988) Nature, 334, 395-402; Davis et al. (1998) Annu Rev Immunol, 16, 523-544).

The interaction of a TCR and a peptide-MHC complex can drive the T cell into various states of activation, depending on the affinity (or dissociation rate) of binding. The TCR recognition process allows a T cell to discriminate between a normal, healthy cell and, for example, one that has become transformed via a virus or malignancy, by providing a diverse repertoire of TCRs, wherein there is a high probability that one or more TCRs will be present with a binding affinity for the foreign peptide bound to an MHC molecule that is above the threshold for stimulating T cell activity (Manning and Kranz (1999) Immunology Today, 20, 417-422).

Wild type TCRs isolated from either human or mouse T cell clones that were identified by in vitro culturing have been shown to have relatively low binding affinities (K_{D}=1 -300 μM) (Davis et al. (1998) Annu Rev Immunol, 16, 523-544). This is partly because that T cells that develop in the thymus are negatively selected (tolerance induction) on self-peptide-MHC ligands, such that T cells with too high of an affinity are deleted (Starr et al. (2003) Annu Rev Immunol, 21, 139-76). To compensate for these relatively low affinities, T cells have evolved a co-receptor system in which the cell surface molecules CD4 and CD8 bind to the MHC molecules (class II and class I, respectively) and synergize with the TCR in mediating signaling activity. CD8 is particularly effective in this process, allowing TCRs with very low affinity (e.g., K_{D}=300 μM) to mediate potent antigen-specific activity.

Directed evolution can be used to generate TCRs with higher affinity for a specific peptide-MHC complex. Methods that can be used include yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), and T cell display (Chervin et al. (2008) J Immunol Methods, 339, 175-84). All three approaches involve engineering, or modifying, a TCR that exhibits the normal, low affinity of the wild-type TCR, to increase the affinity for the cognate peptide-MHC complex (the original antigen that the T cells were specific for).

As such, in some embodiments, the synthetic receptor comprises a TCR, and the fusion proteins provided herein can be co-expressed with a TCR in a cell. In some embodiments, a fusion protein provided herein can be conjugated to a TCR. In some embodiments, the TCR comprises an alpha (α) chain and a beta (β) chain (encoded by TRAC and TRBC, respectively). A human TRAC can have an amino acid sequence corresponding to UniProtKB/Swiss-Prot No.: P01848.2 (Accession: P01848.2 GI: 1431906459). A human TRBC can have an amino acid sequence corresponding to the GenBank sequence ALC78509.1 (Accession: ALC78509.1 GI: 924924895). In some embodiments, the TCR comprises a gamma chain (γ) and a delta (δ) chain (encoded by TRGC and TRDC, respectively). A human TRGC can have an amino acid sequence corresponding to UniProtKB/Swiss-Prot: POCF51.1 (Accession: POCF51.1 GI: 294863156), or an amino acid sequence corresponding to UniProtKB/Swiss-Prot: P03986.2 (Accession: P03986.2 GI: 1531253869). A human TRDC can have an amino acid sequence corresponding to the UniProtKB/Swiss-Prot: B7Z8K6.2 (Accession: B7Z8K6.2 GI: 294863191). The extracellular regions of the αβ chains (or the γδ chains) are responsible for antigen recognition and engagement. Antigen binding stimulates downstream signaling through the multimeric CD3 complex that associates with the intracellular domains of the αβ (or γδ) chains as three dimers (εγ, εδ, ζζ).

TCRs provided herein can be genetically engineered to bind specific antigens. In some embodiments, fusion protein disclosed herein can be co-expressed with a TCR targeting a tumor antigen in a cell. In some embodiments, fusion protein disclosed herein can be conjugated with a TCR targeting a tumor antigen. In some embodiments, the tumor antigen is selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1.

In some embodiments, fusion proteins provided herein can be conjugated to a TCR. In some embodiments, fusion proteins provided herein can be conjugated to a TCR that binds NY-ESO-1. In some embodiments, the amino acid sequence of the TCR that binds NY-ESO-1 is SEQ ID NO:132, which can be encoded by, for example, the nucleotide sequence of SEQ ID NO:133.

In some embodiments, the synthetic receptor does not comprise both the αβ (or γδ) chains of TCR, but can engage and activate endogenous TCR complex. For example, in some embodiments, the synthetic receptor can be a TCR receptor fusion construct (“TRuC”) (Baeuerle et al., Nature Communications 10:2087 (2019)), a T cell antigen coupler (“TAC”) (Helsen et al., Nature communications 9(1): 3049 (2018)), an antibody TCR receptor (“AbTCR”) (Xu et al., Cell Discovery (2018) 4:62), or a chimeric CD3 receptor (WO2016/054520).

In some embodiments, the synthetic receptor is a TRuC (Baeuerle et al., Nature Communications 10:2087 (2019)). In some embodiments, fusion protein disclosed herein can be co-expressed with a TRuC. In some embodiments, fusion protein disclosed herein can be conjugated with a TRuC. TRuCs comprise an antibody-based binding domain fused to T cell receptor (TCR) subunits and can effectively reprogram an intact TCR complex to recognize tumor surface antigens. Unlike CARs, TRuCs become a functional component of the TCR complex. TRuC-T cells have demonstrated potent anti-tumor activity in both liquid and solid tumor xenograft models. To generate a TRuC, the target-binding domain disclosed herein (e.g. a scFV or a single domain antibody) can be fused to the N-terminus of human TCRα, TCRβ, CD3γ, CD3δ, or CD3ε with a linker sequence, thereby providing the TCR and engineered cell with a new target specificity and the potential for HLA-A independent cell lysis. In some embodiments, the target-binding domain is fused to the N-terminus of full-length human TCRα (α-TRuC). In some embodiments, the target-binding domain is fused to the N-terminus of full-length human TCRβ (β-TRuC). In some embodiments, the target-binding domain is fused to the N-terminus of full-length human CD3γ (γ-TRuC). In some embodiments, the target-binding domain is fused to the N-terminus of full-length human CD3δ (δ-TRuC). In some embodiments, the target-binding domain is fused to the N-terminus of full-length human CD3ε (ε-TRuC).

In some embodiments, the synthetic receptor is a TAC (Helsen et al., Nature communications 9(1): 3049 (2018)). In some embodiments, fusion protein disclosed herein can be co-expressed with a TAC. In some embodiments, fusion protein disclosed herein can be conjugated with a TAC. TAC is a chimeric receptor that triggers aggregation and activation of the endogenous TCR following binding to tumor antigens by co-opting the native TCR via the CD3 binding domain, which induces efficient antitumor responses with reduced toxicity when compared with past-generation CARs. TAC design mimics the TCR-CD3:co-receptor complex, and has three components: (1) an antigen-binding domain, (2) a TCR recruitment domain, and (3) a co-receptor domain (hinge, transmembrane, and cytosolic regions). The TAC receptor re-directs the TCR-CD3 complex towards an antigen of choice using an interchangeable antigen binding moiety, uses an scFv to recruit the TCR-CD3 complex, and incorporates co-receptor properties by including the CD4 hinge, TM region, and cytosolic tail. The antigen binding domain can be any target-bind domain disclosed herein. In some embodiments, the antigen binding domain is an anti-HER2 DARPin. In some embodiments, the antigen binding domain is an anti-CD19 scFV. In some embodiments, a TCR recruitment domain is a CD3ε targeting domain. In some embodiments, the CD3ε targeting domain is selected from the group consisting of huUCHT1, UCHT1, F6K, L2K, and OKT3. In some embodiments, the co-receptor domain is a CD4 co-receptor domain. In some embodiments, the co-receptor domain is a CD8α co-receptor domain.

In some embodiments, the synthetic receptor is an AbTCR, which couples the antigen-binding utility of an antibody to the endogenous TCR activation pathways (Xu et al. Cell Discovery (2018) 4:62). In some embodiments, fusion protein disclosed herein can be co-expressed with an AbTCR. In some embodiments, fusion protein disclosed herein can be conjugated with an AbTCR. To generate an AbTCR, an Fab domain of an antibody is combined with the γ and δ chains of the TCR as the effector domain. Specifically, in the AbTCR receptor, the heavy chain domain of the Fab fragment is fused to a portion of the δ chain of a γδ TCR. Whereas, the light chain domain of the Fab fragment is fused to a portion of the γ chain of a γδ TCR. Similar to the exogenously-expressed TCR in TCR-T cells, the AbTCR engages the endogenous CD3 complex to initiate T-cell activation. However, since the γδTCR chains do not bind with the TCRs in αβ T-cells, AbTCR avoids the mispairing challenge of traditional αβTCR-based synthetic receptors. In addition, by employing a Fab as the antigen-binding domain, the AbTCR can be used to target either peptide-MHC complexes or cell surface antigens by using TCR-mimic or conventional antibodies respectively. AbTCR-T cells can trigger antigen-specific cytokine production, degranulation, and killing of cancer cells.

In some embodiments, the synthetic receptor is a chimeric CD3 receptor (WO2016/054520). In some embodiments, fusion protein disclosed herein can be co-expressed with a chimeric CD3 receptor. In some embodiments, fusion protein disclosed herein can be conjugated with a chimeric CD3 receptor. The chimeric CD3 receptors comprise an extracellular domain comprising a CD3 epitope, a transmembrane domain and an intracellular signaling domain. Thus, a cell that expresses the chimeric CD3 receptor can be brought into proximity with a cancer cell expressing a cancer antigen by a bispecific antibody that binds both CD3 and the cancer antigen. In some embodiments, the CD3 epitope is from a CD3R extracellular domain. The intracellular signaling protein is a costimulatory domain, which can comprise a 4-1in signaling domain, a CD28 signaling domain, an ICOS signaling domain, an OX40 signaling domain, a CD27 signaling domain, a CD30 signaling domain, a CD150 signaling domain, a DAP- 10 signaling domain, an NKG2D signaling domain, or a portion or combination thereof. The intracellular domain can further comprise a T cell receptor ζ chain. The chimeric CD3 receptor can further include a transmembrane domain. The transmembrane domain can be a CD8 transmembrane domain, a CD28 transmembrane domain, an FcR transmembrane domain, or a CD3ε transmembrane domain. (WO2016/054520.)

### 6.4 Nucleic Acids

Provided herein are also nucleic acids that encode the fusion proteins disclosed herein. In some embodiments, provided herein are nucleic acids that encode a fusion protein comprising a presenting peptide covalently linked to a β2M peptide via a linker, wherein the fusion protein binds a MHC heavy chain to form an MHC complex that binds an inhibitory receptor of an immune cell to inhibit the immune cell.

In some embodiments, provided herein are nucleic acids that encode a fusion protein comprising an HLA-E-restricted presenting peptide covalently linked to a β2M peptide via a linker. In some embodiments, provided herein are nucleic acids that encode a fusion protein comprising a presenting peptide covalently linked to a β2M peptide via a linker, wherein the presenting peptide is a signal peptide of a Class I MHC molecule, or a fragment thereof. In some embodiments, the fusion protein does not have an MHC heavy chain. In some embodiments, the fusion protein does not have an HLA-E heavy chain. In some embodiments, the fusion protein does not have an HLA-A heavy chain, an HLA-B heavy chain, an HLA-C heavy chain, an HLA-E heavy chain, an HLA-F heavy chain, or an HLA-G heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. Various embodiments of the fusion proteins encoded by the nucleic acids provided herein are provided in Section 6.2 above.

In some embodiments, provided herein are nucleic acids comprising (i) a first fragment encoding a fusion protein comprising a presenting peptide and a β2M peptide covalently linked via a linker, and (ii) a second fragment encoding a synthetic receptor. The synthetic receptor can be any synthetic receptor disclosed herein. In some embodiments, the synthetic receptor can be selected from the group consisting of a chimeric antigen receptor (CAR), a T cell receptor (TCR), a TCR receptor fusion construct (TRuC), a T cell antigen coupler (TAC), an antibody TCR receptor (AbTCR), and a chimeric CD3 receptor.

In some embodiments, the synthetic receptor is a CAR, and nucleic acids provided herein comprise a first fragment encoding a fusion protein as disclosed herein that comprises a presenting peptide and a β2M peptide covalently linked via a linker, and a second fragment encoding a CAR, wherein the second fragment is operatively linked to the first fragment. The CAR can be any CAR disclosed herein or otherwise known in the art. In some embodiments, the CAR comprises an antigen-binding domain that specifically binds a tumor antigen.

In some embodiments, the synthetic receptor is a TCR, and nucleic acids provided herein comprise a first fragment encoding a fusion protein as disclosed herein that comprises a presenting peptide and a β2M peptide covalently linked via a linker, and a second fragment encoding a TCR, wherein the second fragment is operatively linked to the first fragment. The TCR can be any TCR disclosed herein or otherwise known in the art. In some embodiments, the TCR comprises an antigen-binding domain that specifically binds a tumor antigen.

In some embodiments, the synthetic receptor is a TRuc, and nucleic acids provided herein comprise a first fragment encoding a fusion protein as disclosed herein that comprises a presenting peptide and a β2M peptide covalently linked via a linker, and a second fragment encoding a TRuc, wherein the second fragment is operatively linked to the first fragment. In some embodiments, the TRuc comprises an antigen-binding domain that specifically binds a tumor antigen.

In some embodiments, the synthetic receptor is a TAC, and nucleic acids provided herein comprise a first fragment encoding a fusion protein as disclosed herein that comprises a presenting peptide and a β2M peptide covalently linked via a linker, and a second fragment encoding a TAC, wherein the second fragment is operatively linked to the first fragment. In some embodiments, the TAC comprises an antigen-binding domain that specifically binds a tumor antigen.

In some embodiments, the synthetic receptor is an AbTCR, and nucleic acids provided herein comprise a first fragment encoding a fusion protein as disclosed herein that comprises a presenting peptide and a β2M peptide covalently linked via a linker, and a second fragment encoding an AbTCR, wherein the second fragment is operatively linked to the first fragment. In some embodiments, the AbTCR comprises an antigen-binding domain that specifically binds a tumor antigen.

In some embodiments, the synthetic receptor is a chimeric CD3 receptor, and nucleic acids provided herein comprise a first fragment encoding a fusion protein as disclosed herein that comprises a presenting peptide and a β2M peptide covalently linked via a linker, and a second fragment encoding a chimeric CD3 receptor, wherein the second fragment is operatively linked to the first fragment. In some embodiments, the chimeric CD3 receptor comprises an antigen-binding domain that specifically binds a tumor antigen.

In some embodiment, nucleic acids provided herein comprise a first fragment encoding a fusion protein comprising a presenting peptide and a β2M peptide, and a second fragment encoding a synthetic receptor, wherein the first and second fragments are connected via a polynucleotide encoding a self-cleaving peptide. In some embodiments, the first and second fragments are connected via a polynucleotide encoding a 2A peptide. 2A peptides, also known as 2A self-cleaving peptides, include a class of 18-22 amino acids-long peptides and can be used for bicistronic or multicistronic expression of protein sequences (see Szymczak et al., Expert Opin. Biol. Therapy 5(5):627-638 (2005); Liu et al. (2017). Scientific Reports 7 (1): 2193). 2A peptides include, for example, P2A peptide, T2A peptide, F2A peptide, or E2A peptide. An exemplary P2A sequence is (GSG) ATNFSLLKQAGDVEENPGP (SEQ ID NO: 105). An exemplary T2A sequence is (GSG) EGRGSLLTCGDVEENPGP (SEQ ID NO: 106). An exemplary E2A sequence is (GSG) QCTNYALLKLAGDVESNPGP (SEQ ID NO: 107). An exemplary F2A sequence is (GSG) VKQTLNFDLLKLAGDVESNPGP (SEQ ID NO: 108). The sequence “GSG” (Gly-Ser-Gly) on the N-terminal of a 2A peptide is optional.

In some embodiments, nucleic acids provided herein comprise a first fragment encoding a fusion protein comprising a presenting peptide and a β2M peptide, and a second fragment encoding a synthetic receptor, wherein the first and second fragments are connected via a polynucleotide encoding a 2A peptide, and wherein the nucleic acids encode the synthetic receptor, the 2A linker, the fusion protein, from N-terminus to C-terminus. In some embodiments, the nucleic acids provided herein encode the fusion protein, the 2A linker, the synthetic receptor, from N-terminus to C-terminus.

In some embodiments, nucleic acids provided herein comprise a first fragment encoding a fusion protein comprising a presenting peptide and a β2M peptide, and a second fragment encoding a CAR, wherein the first and second fragments are connected via a polynucleotide encoding a 2A peptide, and wherein the nucleic acids encode the CAR, the 2A linker, the fusion protein, from N-terminus to C-terminus. In some embodiments, the nucleic acids provided herein encode the fusion protein, the 2A linker, the CAR, from N-terminus to C-terminus.

In some embodiments, nucleic acids provided herein comprise a first fragment encoding a fusion protein comprising a presenting peptide and a β2M peptide, and a second fragment encoding a synthetic receptor, wherein the first and second fragments are connected via an IRES sequence. An “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990). In some embodiments, nucleic acids provided herein have the first fragment, the IRES sequence, and the second fragment, from 5′ end to 3′ end. In some embodiments, nucleic acids provided herein have the second fragment, the IRES fragment, the first fragment, from 5′ end to 3′ end.

In some embodiments, nucleic acids provided herein encode a fusion protein having an amino acid sequence that is at least 85%, at least 90%, at least 95%, at least 98%, or at least 99% identical to an amino acid sequence selected from the group consisting of SEQ ID NOs: 5 and 13-18. In some embodiments, the amino acid sequence of the fusion protein provided herein comprises an amino acid sequence selected from the group consisting of SEQ ID NOs: 5 and 13-18. Also provided herein are also nucleic acids that hybridize to a nucleic acid encoding an amino acid sequence selected from SEQ ID NOs: 5 and 13-18. In some embodiments, the hybridization is under conditions of high stringency as is known to those skilled in the art.

In some embodiments, the nucleotide sequence of nucleic acids provided herein is at least 85%, at least 90%, at least 95%, at least 98%, or at least 99% identical to SEQ ID NO:6 or 12. In some embodiments, the nucleotide sequence of nucleic acids provided herein is SEQ ID NO:6, or 12.

In some embodiments, the nucleotide sequence of nucleic acid provided herein is at least 85% identical to SEQ ID NO:6. In some embodiments, the nucleotide sequence of nucleic acid provided herein is at least 90% identical to SEQ ID NO:6. In some embodiments, the nucleotide sequence of nucleic acid provided herein is at least 95% identical to SEQ ID NO:6. In some embodiments, the nucleotide sequence of nucleic acid provided herein is at least 98% identical to SEQ ID NO:6. In some embodiments, the nucleotide sequence of nucleic acid provided herein is at least 99% identical to SEQ ID NO:6. In some embodiments, the nucleotide sequence of nucleic acid provided herein is SEQ ID NO:6.

In some embodiments, the nucleotide sequence of nucleic acid provided herein is at least 85% identical to SEQ ID NO:12. In some embodiments, the nucleotide sequence of nucleic acid provided herein is at least 90% identical to SEQ ID NO:12. In some embodiments, the nucleotide sequence of nucleic acid provided herein is at least 95% identical to SEQ ID NO:12. In some embodiments, the nucleotide sequence of nucleic acid provided herein is at least 98% identical to SEQ ID NO: 12. In some embodiments, the nucleotide sequence of nucleic acid provided herein is at least 99% identical to SEQ ID NO: 12. In some embodiments, the nucleotide sequence of nucleic acid provided herein is SEQ ID NO: 12.

Variants of the specific nucleic acids provided herein are also contemplated. The variants can contain alterations in the coding regions, non-coding regions, or both. In some embodiments, a nucleic acid variant contains alterations which produce silent substitutions, additions, or deletions, but does not alter the properties or activities of the encoded polypeptide. In some embodiments, a nucleic acid variant comprises silent substitutions that results in no change to the amino acid sequence of the polypeptide (due to the degeneracy of the genetic code). Nucleic acid variants can be produced for a variety of reasons, for example, to optimize codon expression for a particular host (e.g., change codons in the human mRNA to those preferred by a bacterial host such as E. coli). In some embodiments, a nucleic acid variant comprises at least one silent mutation in a non-coding or a coding region of the sequence.

In some embodiments, a nucleic acid variant is produced to modulate or alter expression (or expression levels) of the encoded nucleic acid. In some embodiments, a nucleic acid variant is produced to increase expression of the encoded polypeptide. In some embodiments, a nucleic acid variant is produced to decrease expression of the encoded polypeptide. In some embodiments, a nucleic acid variant has increased expression of the encoded polypeptide as compared to a parental nucleic acid. In some embodiments, a nucleic acid variant has decreased expression of the encoded polypeptide as compared to a parental nucleic acid.

If desired, nucleic acids provided herein can be codon optimized to increase efficiency of expression of the fusion protein in a given cell. Codon optimization can be used to achieve higher levels of expression in a given cell. Factors that are involved in different stages of protein expression include codon adaptability, mRNA structure, and various cis-elements in transcription and translation. Any suitable codon optimization methods or technologies that are known to one skilled in the art can be used to modify the nucleic acids provided herein. Such codon optimization methods are well known, including commercially available codon optimization services, for example, OptimumGene™ (GenScript; Piscataway, NJ), Encor optimization (EnCor Biotechnology; Gainseville FL), Blue Heron (Blue Heron Biotech; Bothell, WA), and the like. Optionally, multiple codon optimizations can be performed based on different algorithms, and the optimization results blended to generate a codon optimized nucleic acid encoding a polypeptide.

In some embodiments, a nucleic acid is isolated. In some embodiments, a nucleic acid is substantially pure.

Provided herein are also vectors that comprise the nucleic acids provided herein. The vector can be an expression vector. The vector can be a viral vector. In one embodiment, the vector is a retroviral vector, for example, a gamma retroviral vector, which is employed for the introduction of the nucleic acids described herein into a target cell. The vector can be a lentiviral vector. The vector can be an adenoviral vector. The vector can be an adeno-associated viral vector. In some embodiments, the vectors and constructs can optionally be designed to include a reporter.

Nucleic acids provided herein can be prepared, manipulated and/or expressed using any of a variety of well-established techniques known and available in the art. Examples of vectors are plasmid, autonomously replicating sequences, and transposable elements. Exemplary transposon systems such as Sleeping Beauty and PiggyBac can be used, which can be stably integrated into the genome (e.g., Ivics et al., Cell, 91 (4): 501-510 (1997); Cadinanos et al., (2007) Nucleic Acids Research. 35 (12): e87). Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of fusion proteins disclosed herein can be ligated into such expression vectors for their expression in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements can vary in their strength and specificity. Depending on the vector system and host utilized, any number of suitable transcription and translation elements, including ubiquitous promoters and inducible promoters can be used.

Illustrative ubiquitous expression control sequences that can be used in present disclosure include, but are not limited to, a cytomegalovirus (CMV) immediate early promoter, a viral simian virus 40 (SV40) promoter (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken (3-actin (CAG) promoter, and a β-actin promoter.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

### 6.5 Genetically Engineered Cells

Provided herein are genetically engineered cells expressing the fusion proteins disclosed herein. Provided herein are also genetically engineered cells comprising the nucleic acids disclosed herein. In some embodiments, provided herein are also genetically engineered cells comprising the vectors disclosed herein.

In some embodiments, provided herein are genetically engineered cells expressing a fusion protein comprising a presenting peptide covalently linked to a β2M peptide via a linker, wherein the fusion protein binds an MHC heavy chain to form an MHC complex that binds an inhibitory receptor of an immune cell to inhibit the immune cell. In some embodiments, provided herein are genetically engineered cells expressing a fusion protein comprising an HLA-E-restricted presenting peptide covalently linked to a β2M peptide via a linker. In some embodiments, provided herein are genetically engineered cells expressing a fusion protein comprising a presenting peptide covalently linked to a β2M peptide via a linker, wherein the presenting peptide is a signal peptide of a Class I MHC molecule, or a fragment thereof.

In some embodiments, provided herein are genetically engineered cells comprising a nucleic acid encoding a fusion protein comprising a presenting peptide covalently linked to a β2M peptide via a linker, wherein the fusion protein binds an MHC heavy chain to form an MHC complex that binds an inhibitory receptor of an immune cell to inhibit the immune cell. In some embodiments, provided herein are genetically engineered cells comprising a nucleic acid encoding a fusion protein comprising an HLA-E-restricted presenting peptide covalently linked to a β2M peptide via a linker. In some embodiments, provided herein are genetically engineered cells comprising a nucleic acid encoding a fusion protein comprising a presenting peptide covalently linked to a β2M peptide via a linker, wherein the presenting peptide is a signal peptide of a Class I MHC molecule, or a fragment thereof.

In some embodiments, the fusion protein has less than 500 amino acids. In some embodiments, the fusion protein has less than 400, less than 300 amino acids, or less than 200 amino acids. In some embodiments, the fusion protein has about 100 to about 200, about 120 to about 180, about 120 to about 160 amino acids, or about 140 to about 160 amino acids. In some embodiments, the fusion protein lacks an MHC heavy chain. In some embodiments, the fusion protein lacks an HLA-A heavy chain, an HLA-B heavy chain, an HLA-C heavy chain, an HLA-E heavy chain, an HLA-F heavy chain, or an HLA-G heavy chain. In some embodiments, the fusion protein lacks an HLA-E heavy chain. Various embodiments of the fusion proteins are provided in the section above.

The cells disclosed herein are genetically engineered to express a fusion protein that comprises a presenting peptide covalently linked to a β2M peptide via a linker, wherein the fusion protein binds an MHC heavy chain to form a stable MHC complex on the cell surface. In some embodiments, the fusion protein binds an endogenously expressed MHC heavy chain to form a stable MHC complex on the cell surface. In some embodiments, the MHC complex can bind an inhibitory receptor of an immune cell to inhibit the immune cell. As such, provided herein are also stable MHC complexes that comprise the fusion protein disclosed herein and a MHC heavy chain. The MHC heavy chain can be an endogenously expressed.

In some embodiments, provided herein are stable HLA-A complexes that comprise the fusion protein disclosed herein and an HLA-A heavy chain, wherein the fusion protein comprises an HLA-A restricted presenting peptide covalently linked to a β2M peptide via a linker. In some embodiments, provided herein are stable HLA-B complexes that comprise the fusion protein disclosed herein and an HLA-B heavy chain, wherein the fusion protein comprises an HLA-B restricted presenting peptide covalently linked to a β2M peptide via a linker. In some embodiments, provided herein are stable HLA-C complexes that comprise the fusion protein disclosed herein and an HLA-C heavy chain, wherein the fusion protein comprises an HLA-C restricted presenting peptide covalently linked to a β2M peptide via a linker.

In some embodiments, provided herein are stable HLA-E complexes that comprise the fusion protein disclosed herein and an HLA-E heavy chain, wherein the fusion protein comprises an HLA-E restricted presenting peptide covalently linked to a β2M peptide via a linker. In some embodiments, provided herein are stable HLA-F complexes that comprise the fusion protein disclosed herein and an HLA-F heavy chain, wherein the fusion protein comprises an HLA-F restricted presenting peptide covalently linked to a β2M peptide via a linker. In some embodiments, provided herein are stable HLA-G complexes that comprise the fusion protein disclosed herein and an HLA-G heavy chain, wherein the fusion protein comprises an HLA-G restricted presenting peptide covalently linked to a β2M peptide via a linker.

In some embodiments, provided herein are stable CD1 complexes that comprise the fusion protein disclosed herein and a CD1 heavy chain, wherein the fusion protein comprises a CD1 restricted presenting molecule covalently linked to a β2M peptide via a linker. In some embodiments, provided herein are stable MR1 complexes that comprise the fusion protein disclosed herein and an MR1 heavy chain, wherein the fusion protein comprises an MR1 restricted presenting molecule covalently linked to a β2M peptide via a linker. In some embodiments, provided herein are stable FcRn complexes that comprise the fusion protein disclosed herein and an FcRn heavy chain, wherein the fusion protein comprises an FcRn restricted presenting molecule covalently linked to a β2M peptide via a linker. In some embodiments, provided herein are stable UL18 complexes that comprise the fusion protein disclosed herein and a UL18 protein, wherein the fusion protein comprises a UL18 restricted presenting peptide covalently linked to a β2M peptide via a linker.

The formation of an MHC complex can be detected by any methods disclosed herein or otherwise known in the art. For example, the co-localization of the fusion protein disclosed herein and the MHC heavy chain in the MHC complex can be detected using β2M co-immunoprecipitation (Lee et al., J Immunol 1998; 160: 4951-60). For another example, the interaction of MHC heavy chain and the fusion protein disclosed herein in the MHC complex can be detected by surface plasmon resonance technology (SPR) (Goodridge et al., J. Immunol. 2010; 184(11): 6199-6208). For yet another example, the fusion protein and the MHC heavy chain can each be labeled with different fluorescent labels, and the co-localization of MHC heavy chain and the fusion protein disclosed herein in the MHC complex can be detected by microscopy or flow cytometry. A person of ordinary skill in the art would understand that a variety of assays that are used to detect protein-protein interactions can be used for detecting the MHC complex, including, for example, pull-down assays, crosslinking, label transfer, far-western blot analysis, protein affinity purification, two hybrid assays (yeast, mammalian), Forster Resonance Energy Transfer (FRET), gel-shift assays, etc. (See e.g. Berggård et al. Proteomics 2007, 7, 2833-2842; Phizicky et al. (1995) Microbiol Rev. 59:94-123; Golemis E (2002) Protein-protein interactions: A molecular cloning manual. Cold Spring Harbor (NY): Cold Spring Harbor Laboratory Press. p ix, 682.)

In some embodiments, the genetically engineered cells disclosed herein further express a synthetic receptor that comprises (1) a target-binding domain that can specifically bind a target molecule, and (2) a functional domain that can activate a signaling pathway in the engineered cell. The synthetic receptor can be a CAR, a TCR, a TRuC, a TAC, an AbTCR, or a chimeric CD3 receptor. In some embodiments, the genetically engineered cells disclosed herein further express a CAR. In some embodiments, the genetically engineered cells disclosed herein further express a TCR. In some embodiments, the genetically engineered cells disclosed herein further express a TRuC. In some embodiments, the genetically engineered cells disclosed herein further express a TAC. In some embodiments, the genetically engineered cells disclosed herein further express an AbTCR. In some embodiments, the genetically engineered cells disclosed herein further express a chimeric CD3 receptor.

In some embodiments, the synthetic receptor is a CAR. The CAR can be any CAR disclosed herein or otherwise known in the art. In some embodiments, the CAR comprises an antigen-binding domain that specifically binds a tumor antigen. As such, in some embodiments, provided herein are also genetically engineered cells expressing a fusion protein disclosed herein and a CAR. In some embodiments, genetically engineered cells provided herein comprise a nucleic acid that comprises a first fragment encoding a fusion protein, and a second fragment encoding a CAR. The first and second fragments of the nucleic acid can be connected via a polynucleotide encoding a 2A peptide. The first and second fragments of the nucleic acid can also be connected with an IRES sequence. In some embodiments, genetically engineered cells provided herein comprise a nucleic acid encoding a fusion protein comprising a presenting peptide and a β2M peptide covalently linked via a linker, and a second nucleic acid encoding a CAR.

In some embodiments, the synthetic receptor is a TCR. The TCR can be any TCR disclosed herein or otherwise known in the art. In some embodiments, the TCR comprises an antigen-binding domain that specifically binds a tumor antigen. As such, in some embodiments, provided herein are also genetically engineered cells expressing a fusion protein disclosed herein and a TCR. In some embodiments, genetically engineered cells provided herein comprise a nucleic acid that comprises a first fragment encoding a fusion protein, and a second fragment encoding a TCR. The first and second fragments of the nucleic acid can be connected via a polynucleotide encoding a 2A peptide. The first and second fragments of the nucleic acid can also be connected with an IRES sequence. In some embodiments, genetically engineered cells provided herein comprise a nucleic acid encoding a fusion protein comprising a presenting peptide and a β2M peptide covalently linked via a linker, and a second nucleic acid encoding a TCR.

In some embodiments, the synthetic receptor is a TRuC. The TRuC can be any TRuC disclosed herein or otherwise known in the art. In some embodiments, the TRuC comprises an antigen-binding domain that specifically binds a tumor antigen. As such, in some embodiments, provided herein are also genetically engineered cells expressing a fusion protein disclosed herein and a TRuC. In some embodiments, genetically engineered cells provided herein comprise a nucleic acid that comprises a first fragment encoding a fusion protein, and a second fragment encoding a TRuC. The first and second fragments of the nucleic acid can be connected via a polynucleotide encoding a 2A peptide. The first and second fragments of the nucleic acid can also be connected with an IRES sequence. In some embodiments, genetically engineered cells provided herein comprise a nucleic acid encoding a fusion protein comprising a presenting peptide and a β2M peptide covalently linked via a linker, and a second nucleic acid encoding a TRuC.

In some embodiments, the synthetic receptor is a TAC. The TAC can be any TAC disclosed herein or otherwise known in the art. In some embodiments, the TAC comprises an antigen-binding domain that specifically binds a tumor antigen. As such, in some embodiments, provided herein are also genetically engineered cells expressing a fusion protein disclosed herein and a TAC. In some embodiments, genetically engineered cells provided herein comprise a nucleic acid that comprises a first fragment encoding a fusion protein, and a second fragment encoding a TAC. The first and second fragments of the nucleic acid can be connected via a polynucleotide encoding a 2A peptide. The first and second fragments of the nucleic acid can also be connected with an IRES sequence. In some embodiments, genetically engineered cells provided herein comprise a nucleic acid encoding a fusion protein comprising a presenting peptide and a β2M peptide covalently linked via a linker, and a second nucleic acid encoding a TAC.

In some embodiments, the synthetic receptor is an AbTCR. The AbTCR can be any AbTCR disclosed herein or otherwise known in the art. In some embodiments, the AbTCR comprises an antigen-binding domain that specifically binds a tumor antigen. As such, in some embodiments, provided herein are also genetically engineered cells expressing a fusion protein disclosed herein and an AbTCR. In some embodiments, genetically engineered cells provided herein comprise a nucleic acid that comprises a first fragment encoding a fusion protein, and a second fragment encoding an AbTCR. The first and second fragments of the nucleic acid can be connected via a polynucleotide encoding a 2A peptide. The first and second fragments of the nucleic acid can also be connected with an IRES sequence. In some embodiments, genetically engineered cells provided herein comprise a nucleic acid encoding a fusion protein comprising a presenting peptide and a β2M peptide covalently linked via a linker, and a second nucleic acid encoding an AbTCR.

In some embodiments, the synthetic receptor is a chimeric CD3 receptor. The chimeric CD3 receptor can be any chimeric CD3 receptor disclosed herein or otherwise known in the art. In some embodiments, the chimeric CD3 receptor comprises an antigen-binding domain that specifically binds a tumor antigen. As such, in some embodiments, provided herein are also genetically engineered cells expressing a fusion protein disclosed herein and a chimeric CD3 receptor. In some embodiments, genetically engineered cells provided herein comprise a nucleic acid that comprises a first fragment encoding a fusion protein, and a second fragment encoding a chimeric CD3 receptor. The first and second fragments of the nucleic acid can be connected via a polynucleotide encoding a 2A peptide. The first and second fragments of the nucleic acid can also be connected with an IRES sequence. In some embodiments, genetically engineered cells provided herein comprise a nucleic acid encoding a fusion protein comprising a presenting peptide and a β2M peptide covalently linked via a linker, and a second nucleic acid encoding a chimeric CD3 receptor.

In some embodiments, the cells provided herein are also engineered to reduce the endogenous expression of a Class I MHC molecule or an MHC-like molecule on cell surface. The inhibition of expression of Class I MHC molecules and/or MHC-like molecules on the surface of donor cells can reduce or eliminate allogeneic antigen presentation on the donor cells and help escape the recognition and clearance of the donor cell by host T cells, and is therefore advantageous for improving the compatibility of allografts. For example, in the context of CAR-T therapy, it has been suggested that removal of Class I MHC molecules from the donor T cell can eliminate the “non-self” antigen presentation of the allogeneic donor cell and therefore protect it from the host immune system, especially CD8+ T cells (WO2015136001).

In some embodiments, the cells provided herein are engineered to reduce the endogenous expression of a Class I MHC molecule or an MHC-like molecule. In some embodiments, the cells disclosed herein lack endogenous expression of a Class I MHC molecule or an MHC-like molecule on the cell surface. In some embodiments, the cells disclosed herein lack endogenous expression of a Class I MHC molecule on the cell surface. In some embodiments, the cells disclosed herein lack endogenous expression of a classical Class I MHC molecule on the cell surface. In some embodiments, the cells disclosed herein lack endogenous expression of a nonclassical Class I MHC molecule on the cell surface. In some embodiments, the cells disclosed herein lack endogenous expression of an MHC-like molecule on the cell surface. In some embodiments, the cells disclosed herein lack endogenous expression of a heavy chain of HLA-A, HLA-B, HLA-C, HLA-E, HLA-F, HLA-G, or any combination thereof on the cell surface. In some embodiments, the cells disclosed herein lack endogenous expression of HLA-A heavy chain on the cell surface. In some embodiments, the cells disclosed herein lack endogenous expression of HLA-B heavy chain on the cell surface. In some embodiments, the cells disclosed herein lack endogenous expression of HLA-C heavy chain on the cell surface. In some embodiments, the cells disclosed herein lack endogenous expression of HLA-E heavy chain on the cell surface. In some embodiments, the cells disclosed herein lack endogenous expression of HLA-F heavy chain on the cell surface. In some embodiments, the cells disclosed herein lack endogenous expression of HLA-G heavy chain on the cell surface. In some embodiments, the cells disclosed herein lack endogenous expression of HLA-A heavy chain, HLA-B heavy chain, and HLA-C heavy chain on the cell surface. In some embodiments, the cells disclosed herein lack endogenous expression of HLA-E heavy chain, HLA-F heavy chain, and HLA-G heavy chain on the cell surface. In some embodiments, the cells disclosed herein lack endogenous expression of heavy chains of HLA-A, HLA-B, HLA-C, HLA-E, HLA-F, and HLA-G on the cell surface. In some embodiments, the cells disclosed herein lack endogenous expression of at least an MHC-like molecule on the cell surface. In some embodiments, the cells disclosed herein lack endogenous expression of CD1 heavy chain, MR1 heavy chain, FcRN heavy chain, UL18, or any combination thereof on the cell surface. In some embodiments, the cells disclosed herein lack endogenous expression of CD1 heavy chain on the cell surface. In some embodiments, the cells disclosed herein lack endogenous expression of MR1 heavy chain on the cell surface. In some embodiments, the cells disclosed herein lack endogenous expression of FcRn heavy chain on the cell surface. In some embodiments, the cells disclosed herein lack endogenous expression of UL18 on the cell surface. In some embodiments, the cells disclosed herein lack endogenous expression of CD1 heavy chain, MR1 heavy chain, FcRn heavy chain and UL18 on the cell surface.

In some embodiments, the cells disclosed herein lack endogenous expression of β2M on the cell surface. Because β2M is an indispensable structural component of a variety of MHC complexes having different heavy chains, inhibition of β2M expression can lead to a reduction or elimination of Class I MHC molecules and some MHC-like molecules on the surface of the engineered cell. In some embodiments, the cells disclosed herein are genetically engineered to (1) express a fusion protein disclosed herein that comprises a presenting peptide covalently linked to a β2M peptide via a linker, and (2) lack endogenous expression of β2M. The fusion protein can be any fusion protein disclosed herein. In some embodiments, the fusion protein comprises an HLA-E-restricted presenting peptide, and forms a complex with an endogenous HLA-E heavy chain that can be recognized by an inhibitory receptor of an immune cell, such as the NKG2A receptor on NK cells.

When the endogenous expression of β2M is eliminated, the formation of the MHC complex can be measured by co-localization of the fusion protein and the heavy chain on the cell surface, and the MHC complex comprises the fusion protein disclosed herein, which can be detected by using an anti-β2M antibody, and the endogenous MHC heavy chain, which can be detected by an antibody that recognizes the heavy chain. For example, the antibody clone W6/32 can be used to detect HLA-ABC; the antibody clone 3D12 can be used to detect HLA-E; and the antibody clone TU99 can be used to detect β2M.

Cells disclosed herein that lack endogenous expression of a Class I MHC molecule or an MHC-like molecule on the cell surface means that the level of endogenous expression of the molecule at the cell surface is reduced significantly compared to a non-engineered cell. In some embodiments, the endogenous expression of the molecule at the cell surface is reduced by at least 50% compared to a non-engineered cell. In some embodiments, the endogenous expression of the molecule at the cell surface is reduced by at least 60% compared to a non-engineered cell. In some embodiments, the endogenous expression of the molecule at the cell surface is reduced by at least 70% compared to a non-engineered cell. In some embodiments, the endogenous expression of the molecule at the cell surface is reduced by at least 80% compared to a non-engineered cell. In some embodiments, the endogenous expression of the molecule at the cell surface is reduced by at least 90% compared to a non-engineered cell. In some embodiments, the endogenous expression of the molecule at the cell surface is reduced by at least 95% compared to a non-engineered cell. In some embodiments, the endogenous expression of the molecule at the cell surface is not detectable. For example, in some embodiments, the Class I MHC molecule or MHC-like molecule cannot be detected by Western Blot in the cell membrane fraction of the cells disclosed herein, or cannot be detected by immunofluorescence on the cell membrane of cells. In some embodiments, the cells provided herein are engineered to delete (or knock out) the gene encoding the Class I MHC molecule or MHC-like molecule.

In some embodiments, provided herein are a population of genetically engineered cells described herein, wherein at least 50% cells lack endogenous expression of a Class I MHC molecule or an MHC-like molecule on the cell surface. In some embodiments, provided herein are a population of genetically engineered cells described herein, wherein at least 60% cells lack endogenous expression of a Class I MHC molecule or an MHC-like molecule on the cell surface. In some embodiments, provided herein are a population of genetically engineered cells described herein, wherein at least 75% cells lack endogenous expression of a Class I MHC molecule or an MHC-like molecule on the cell surface. In some embodiments, provided herein are a population of genetically engineered cells described herein, wherein at least 80% cells lack endogenous expression of a Class I MHC molecule or an MHC-like molecule on the cell surface. In some embodiments, provided herein are a population of genetically engineered cells described herein, wherein at least 85% cells lack endogenous expression of a Class I MHC molecule or an MHC-like molecule on the cell surface. In some embodiments, provided herein are a population of genetically engineered cells described herein, wherein at least 90% cells lack endogenous expression of a Class I MHC molecule or an MHC-like molecule on the cell surface. In some embodiments, provided herein are a population of genetically engineered cells described herein, wherein at least 95% cells lack endogenous expression of a Class I MHC molecule or an MHC-like molecule on the cell surface.

The genetically engineered cell provided herein can be of any type. In some embodiments, the cell is suitable for transplantation. In some embodiments, the cell is suitable for allogeneic transplantation. In some embodiments, the cell is selected from the group consisting of a stem cell, a pluripotent cell, a progenitor cells, a hematopoietic stem and/or progenitor cell, a CD34+ mobilized peripheral blood cell, a CD34+ cord blood cell, a CD34+ bone marrow cell, a hepatocyte, a somatic cell, an immune cell, and a non-transformed cell. In some embodiments, the cell is a stem cell. In some embodiments, the cell is a pluripotent cell. In some embodiments, the cell is a progenitor cell. In some embodiments, the cell is a hematopoietic stem and/or progenitor cell. In some embodiments, the cell is a CD34+ mobilized peripheral blood cell. In some embodiments, the cell is a CD34+ cord blood cell. In some embodiments, the cell is a CD34+ bone marrow cell. In some embodiments, the cell is a hepatocyte. In some embodiments, the cell is a somatic cell. In some embodiments, the cell is a non-transformed cell.

In some embodiments, the cell provided herein is a stem cell. The cell can be a somatic cell. The cell can be a non-pluripotent cell. The cell can be an incompletely or partially pluripotent stem cell. The cell can be a multipotent cell. The cell can be an oligopotent cell, The cell can be a unipotent cell. The cell can be a terminally differentiated cell. Pluripotent cells suitable for use in particular embodiments include, but are not limited to, naturally-occurring stem cells, embryonic stem cells, or induced pluripotent cells (iPSCs). Provided herein are also a mixed population of cells combining any of the cells mentioned above. For example, a population of cells provided herein can comprise cells undergoing reprogramming, which comprise pluripotent cells, partially pluripotent cells, and non-pluripotent cells, such as fully differentiated cells.

In some embodiments, the cell provided herein is an adult stem/progenitor cell. In some embodiments, the cell provided herein is a neonatal stem/progenitor cell. In some embodiments, the cell is selected from the group consisting of: a mesodermal stem/progenitor cell, an endodermal stem/progenitor cell, and an ectodermal stem/progenitor cell. In some embodiments, the cell is a mesodermal stem/progenitor cell. In some embodiments, the cell is an endodermal stem/progenitor cell. In some embodiments, the cell is an ectodermal stem/progenitor cell.

Illustrative examples of mesodermal stem/progenitor cells include, but are not limited to: mesodermal stem/progenitor cells, endothelial stem/progenitor cells, bone marrow stem/progenitor cells, umbilical cord stem/progenitor cells, adipose tissue derived stem/progenitor cells, hematopoietic stem/progenitor cells (HSCs), mesenchymal stem/progenitor cells, muscle stem/progenitor cells, kidney stem/progenitor cells, osteoblast stem/progenitor cells, chondrocyte stem/progenitor cells, and the like. In some embodiments, the cell is a mesodermal stem/progenitor cell. In some embodiments, the cell is an endothelial stem/progenitor cell. In some embodiments, the cell is a bone marrow stem/progenitor cell. In some embodiments, the cell is an umbilical cord stem/progenitor cell. In some embodiments, the cell is an adipose tissue derived stem/progenitor cell. In some embodiments, the cell is a hematopoietic stem/progenitor cell (HSC). In some embodiments, the cell is a mesenchymal stem/progenitor cell. In some embodiments, the cell is a muscle stem/progenitor cell. In some embodiments, the cell is a kidney stem/progenitor cell. In some embodiments, the cell is an osteoblast stem/progenitor cell. In some embodiments, the cell is a chondrocyte stem/progenitor cell.

Illustrative examples of ectodermal stem/progenitor cells include, but are not limited to, neural stem/progenitor cells, retinal stem/progenitor cells, skin stem/progenitor cells, and the like. In some embodiments, the cell is a neural stem/progenitor cell. In some embodiments, the cell is a retinal stem/progenitor cell. In some embodiments, the cell is a skin stem/progenitor cell.

Illustrative examples of endodermal stem/progenitor cells include, but are not limited to, liver stem/progenitor cells, pancreatic stem/progenitor cells, epithelial stem/progenitor cells, and the like. In some embodiments, the cell is an endodermal stem/progenitor cell. In some embodiments, the cell is a liver stem/progenitor cell. In some embodiments, the cell is a pancreatic stem/progenitor cell. In some embodiments, the cell is an epithelial stem/progenitor cell.

In certain embodiments, the cell provided herein is selected from the group consisting of: pancreatic islet cells, CNS cells, PNS cells, cardiac muscle cells, skeletal muscle cells, smooth muscle cells, hematopoietic cells, bone cells, liver cells, an adipose cells, renal cells, lung cells, chondrocyte, skin cells, follicular cells, vascular cells, epithelial cells, immune cells, endothelial cells, and the like. In some embodiments, the cell is a pancreatic islet cell. In some embodiments, the cell is a CNS cell. In some embodiments, the cell is a PNS cell. In some embodiments, the cell is a cardiac muscle cell. In some embodiments, the cell is a skeletal muscle cell. In some embodiments, the cell is a smooth muscle cell. In some embodiments, the cell is a hematopoietic cell. In some embodiments, the cell is a bone cell. In some embodiments, the cell is a liver cell. In some embodiments, the cell is an adipose cell. In some embodiments, the cell is a renal cell. In some embodiments, the cell is a lung cell. In some embodiments, the cell is a chondrocyte. In some embodiments, the cell is a skin cell. In some embodiments, the cell is a follicular cell. In some embodiments, the cell is a vascular cell. In some embodiments, the cell is an epithelial cell. In some embodiments, the cell is am endothelial cell.

In some embodiments, the cell provided herein is an immune cell. The cell can be a leukocyte. A leukocyte is a karyocyte developed from a hematopoietic stem cell and plays important roles in the hematopoietic system and the lymphatic system. Leukocytes includes myeloid cells, lymphoid cells, granulocytes (such as neutrophils, eosinophils, basophils), lymphocytes (such as T cells, B cells, NK cells, NKT cells), plasma cells, dendritic cells, monocytes and cells differentiated therefrom such as a macrophage. In some embodiments, the cell provided herein is a leukocyte. In some embodiments, the cell provided herein is a myeloid cell. In some embodiments, the cell provided herein is a lymphoid cell. In some embodiments, the cell provided herein is a granulocyte. In some embodiments, the cell provided herein is a neutrophil. In some embodiments, the cell provided herein is an eosinophil. In some embodiments, the cell provided herein is a basophil. In some embodiments, the cell provided herein is a T cell. In some embodiments, the cell provided herein is a B cell. In some embodiments, the cell provided herein is a plasma cell. In some embodiments, the cell provided herein is an NK cell. In some embodiments, the cell provided herein is an NKT cell. In some embodiments, the cell provided herein is a dendritic cell. In some embodiments, the cell provided herein is a monocyte. In some embodiments, the cell provided herein is a macrophage.

In some embodiments, the cell provided herein is a tumor-infiltrating lymphocyte (TIL).

In some embodiments, provided herein are a population of the cells disclosed herein. The population of cells can be a homogenous population of cells. The population of cells can be a heterogeneous population of cells. In some embodiments, the population of cells can be a heterogeneous population of cells comprising any combination of the cells disclosed herein.

T cells provided herein can be used in allogeneic transplantation. For example, some CAR-T cells disclosed herein can be universal CAR-Ts that can be used in allogeneic transplantation for cancer treatment. In some embodiments, provided herein is a genetically engineered T cell that expresses the fusion protein disclosed herein. In some embodiments, provided herein is a genetically engineered T cell that comprises the nucleic acid disclosed herein. In some embodiments, provided herein is a CAR-T cell.

In some embodiments, the genetically engineered cell provided herein is a universal lymphocyte, namely, a lymphocyte that can be applied to allotransplantation, which either does not produce or produces a controlled GvHD reaction. A GvHD (graft-versus-host disease) reaction refers to the medical complication following transplantation of tissues having immunologically active cells, wherein the immunologically active cells in the graft tissue recognize the recipient's healthy tissue as foreign and initiate an immune response against the recipient's healthy tissues. GvHD can be clinically controlled by drug intervention and does not threaten life; but severe GvHD can be life-threatening. GvHD is a particular concern for allogeneic CAR-T therapies, as the allogeneic T cells might recognize not only the cancer but also the noncancerous tissues in the recipient as non-self and attack such noncancerous tissues.

A T-Cell Receptor (TCR) is a transmembrane octamer on T cells composed of a TCR dimer and various subunits of CD3. TCR is responsible for antigen recognition by T cells, which is required for T cell activation. Components of the TCR receptor include α and β subunits of TCR, and 6, F, 7, C or f subunits of CD3. Removal of a functional TCR component from the donor T cell can prevent the donor T cell from attacking host cells. Accordingly, in some embodiments, T cells provided herein also lack the expression of a component of the TCR complex. In some embodiments, at least one gene encoding a component of the TCR is inactivated in the T cells provided herein. In some embodiments, the gene encoding α subunit of TCR (e.g. TRAC) is inactivated in the T cells provided herein. A human TRAC can have an amino acid sequence corresponding to UniProtKB/Swiss-Prot No.: P01848.2 (Accession: P01848.2 GI: 1431906459). In some embodiments, the gene encoding β subunit of TCR (e.g. TRBC) is inactivated in the T cells provided herein. A human TRBC can have an amino acid sequence corresponding to the GenBank sequence ALC78509.1 (Accession: ALC78509.1 GI: 924924895).

In some embodiments, the gene encoding the 6 subunit of CD3 is inactivated in the T cells provided herein. A human 6 subunit of CD3 can have an amino acid sequence corresponding to the sequence having NCBI Reference Sequence: NP_000723.1 (Accession: NP_000723.1 GI: 4502669; Isoform A) or NCBI Reference Sequence: NP_001035741.1 (Accession: NP_001035741.1 GI: 98985801; Isoform B). In some embodiments, the gene encoding the F subunit of CD3 is inactivated in the T cells provided herein. A human F subunit of CD3 can have an amino acid sequence corresponding to the sequence having UniProtKB/Swiss-Prot: P07766.2 (Accession: P07766.2 GI: 1345708). In some embodiments, the gene encoding the γ subunit of CD3 is inactivated in the T cells provided herein. A human γ subunit of CD3 can have an amino acid sequence corresponding to the sequence having NCBI Reference Sequence: NP_000064.1 (Accession: NP_000064.1 GI: 4557429). In some embodiments, the gene encoding the ζ subunit of CD3 is inactivated in the T cells provided herein. A human the ζ subunit of CD3 can have an amino acid sequence corresponding to the sequence having UniProtKB/Swiss-Prot: P20963.1 (Accession: P20963.1 GI: 23830999). In some embodiments, the gene encoding the η subunit of CD3 is inactivated in the T cells provided herein. A human η subunit of CD3 can have an amino acid sequence corresponding to the sequence having UniProt Identifier P20963-2.

For illustrative purpose, in some embodiments, provided herein are genetically engineered T cells expressing the fusion protein comprising a presenting peptide covalently linked to a β2M peptide via a linker, wherein the T cells further express a CAR, and wherein the endogenous genes encoding β2M and α subunit of TCR are inactivated in the T cells. The fusion protein can be any fusion protein disclosed herein. The CAR can be any CAR disclosed herein or otherwise known in the art. In some embodiments, the fusion protein comprises human β2M covalently linked to a presenting peptide via a (G4S)3 linker, wherein the presenting peptide is HLA-E restricted and is an 8-10 amino acid fragment of a signal peptide of a Class I MHC molecule. In some embodiments, the CAR comprises an antigen-binding domain that specifically binds a tumor antigen (e.g. CD19).

In some embodiments, the cells provided herein are genetically engineered T cells expressing a fusion protein having an amino acid sequence selected from the group consisting of SEQ ID NOs: 5 and 13-18. In some embodiments, the T cells further express a CAR having an antigen-binding domain that specifically binds CD19 or BCMA. In some embodiments, the amino acid sequence of the CAR targeting CD19 is selected from the group consisting of SEQ ID NOs:74-76. In some embodiments, the amino acid sequence of the CAR targeting BCMA is selected from the group consisting of SEQ ID NOs:77-80 and 136. In some embodiments, the T cells further express a TCR having an antigen-binding domain that specifically binds NY-ESO-1. In some embodiments, the TCR targeting NY-ESO-1 has the amino acid sequence of SEQ ID NO:132. In some embodiments, the T cells comprises a nucleic acid that comprises a first fragment encoding a fusion protein, and a second fragment encoding a CAR or TCR. In some embodiments, the T cells comprises a nucleic acid that encodes a fusion protein having an amino acid sequence selected from the group consisting of SEQ ID NOs:5 and 13-18, and a CAR having an amino acid sequence selected from the group consisting of SEQ ID NOs:74-80 and 136. In some embodiments, the T cells comprises a nucleic acid that encodes a fusion protein having an amino acid sequence selected from the group consisting of SEQ ID NOs:5 and 13-18, and a TCR having the amino acid sequence of SEQ ID NO:132. In some embodiments, the T cells comprises a nucleic acid that encodes an amino acid sequence that is at least 85%, at least 90%, at least 95%, at least 98%, or at least 99% identical to an amino acid sequence selected from the group consisting of SEQ ID NOs:11, 124, 126, 128, 130, 134, and 138. In some embodiments, the T cells comprises a nucleic acid that encodes an amino acid sequence selected from the group consisting of SEQ ID NOs:11, 124, 126, 128, 130, 134, and 138. In some embodiments, the endogenous genes encoding β2M and α subunit of TCR are inactivated in the T cells. In some embodiments, the endogenous genes encoding β2M and α subunit of TCR are knocked out in the T cells.

In some embodiments, the T cells comprises a nucleic acid that encodes an amino acid sequence that is at least 85% identical to SEQ ID NO:11. In some embodiments, the T cells comprises a nucleic acid that encodes an amino acid sequence that is at least 90% identical to SEQ ID NO:11. In some embodiments, the T cells comprises a nucleic acid that encodes an amino acid sequence that is at least 95% identical to SEQ ID NO:11. In some embodiments, the T cells comprises a nucleic acid that encodes an amino acid sequence that is at least 98% identical to SEQ ID NO:11. In some embodiments, the T cells comprises a nucleic acid that encodes an amino acid sequence that is at least 99% identical to SEQ ID NO:11. In some embodiments, the T cells comprises a nucleic acid that encodes the amino acid sequence of SEQ ID NO:11. In some embodiments, the T cells comprises a nucleic acid that is at least 85%, at least 90%, at least 95%, at least 98%, or at least 99% identical to SEQ ID NO:12. In some embodiments, the T cells comprises a nucleic acid having the nucleotide sequence of SEQ ID NO:12. In some embodiments, the endogenous genes encoding β2M and α subunit of TCR are inactivated in the T cells. In some embodiments, the endogenous genes encoding β2M and α subunit of TCR are knocked out in the T cells.

In some embodiments, the T cells comprises a nucleic acid that encodes an amino acid sequence that is at least 85% identical to SEQ ID NO:124. In some embodiments, the T cells comprises a nucleic acid that encodes an amino acid sequence that is at least 90% identical to SEQ ID NO:124. In some embodiments, the T cells comprises a nucleic acid that encodes an amino acid sequence that is at least 95% identical to SEQ ID NO:124. In some embodiments, the T cells comprises a nucleic acid that encodes an amino acid sequence that is at least 98% identical to SEQ ID NO:124. In some embodiments, the T cells comprises a nucleic acid that encodes an amino acid sequence that is at least 99% identical to SEQ ID NO:124. In some embodiments, the T cells comprises a nucleic acid that encodes the amino acid sequence of SEQ ID NO:124. In some embodiments, the T cells comprises a nucleic acid that is at least 85%, at least 90%, at least 95%, at least 98%, or at least 99% identical to SEQ ID NO:125. In some embodiments, the T cells comprises a nucleic acid having the nucleotide sequence of SEQ ID NO:125. In some embodiments, the endogenous genes encoding β2M and α subunit of TCR are inactivated in the T cells. In some embodiments, the endogenous genes encoding β2M and α subunit of TCR are knocked out in the T cells.

In some embodiments, the T cells comprises a nucleic acid that encodes an amino acid sequence that is at least 85% identical to SEQ ID NO:126. In some embodiments, the T cells comprises a nucleic acid that encodes an amino acid sequence that is at least 90% identical to SEQ ID NO:126. In some embodiments, the T cells comprises a nucleic acid that encodes an amino acid sequence that is at least 95% identical to SEQ ID NO:126. In some embodiments, the T cells comprises a nucleic acid that encodes an amino acid sequence that is at least 98% identical to SEQ ID NO:126. In some embodiments, the T cells comprises a nucleic acid that encodes an amino acid sequence that is at least 99% identical to SEQ ID NO:126. In some embodiments, the T cells comprises a nucleic acid that encodes the amino acid sequence of SEQ ID NO:126. In some embodiments, the T cells comprises a nucleic acid that is at least 85%, at least 90%, at least 95%, at least 98%, or at least 99% identical to SEQ ID NO:127. In some embodiments, the T cells comprises a nucleic acid having the nucleotide sequence of SEQ ID NO:127. In some embodiments, the endogenous genes encoding β2M and α subunit of TCR are inactivated in the T cells. In some embodiments, the endogenous genes encoding β2M and α subunit of TCR are knocked out in the T cells.

In some embodiments, the T cells comprises a nucleic acid that encodes an amino acid sequence that is at least 85% identical to SEQ ID NO:128. In some embodiments, the T cells comprises a nucleic acid that encodes an amino acid sequence that is at least 90% identical to SEQ ID NO:128. In some embodiments, the T cells comprises a nucleic acid that encodes an amino acid sequence that is at least 95% identical to SEQ ID NO:128. In some embodiments, the T cells comprises a nucleic acid that encodes an amino acid sequence that is at least 98% identical to SEQ ID NO:128. In some embodiments, the T cells comprises a nucleic acid that encodes an amino acid sequence that is at least 99% identical to SEQ ID NO:128. In some embodiments, the T cells comprises a nucleic acid that encodes the amino acid sequence of SEQ ID NO:128. In some embodiments, the T cells comprises a nucleic acid that is at least 85%, at least 90%, at least 95%, at least 98%, or at least 99% identical to SEQ ID NO:129. In some embodiments, the T cells comprises a nucleic acid having the nucleotide sequence of SEQ ID NO:129. In some embodiments, the endogenous genes encoding β2M and α subunit of TCR are inactivated in the T cells. In some embodiments, the endogenous genes encoding β2M and α subunit of TCR are knocked out in the T cells.

In some embodiments, the T cells comprises a nucleic acid that encodes an amino acid sequence that is at least 85% identical to SEQ ID NO:130. In some embodiments, the T cells comprises a nucleic acid that encodes an amino acid sequence that is at least 90% identical to SEQ ID NO:130. In some embodiments, the T cells comprises a nucleic acid that encodes an amino acid sequence that is at least 95% identical to SEQ ID NO:130. In some embodiments, the T cells comprises a nucleic acid that encodes an amino acid sequence that is at least 98% identical to SEQ ID NO:130. In some embodiments, the T cells comprises a nucleic acid that encodes an amino acid sequence that is at least 99% identical to SEQ ID NO:130. In some embodiments, the T cells comprises a nucleic acid that encodes the amino acid sequence of SEQ ID NO:130. In some embodiments, the T cells comprises a nucleic acid that is at least 85%, at least 90%, at least 95%, at least 98%, or at least 99% identical to SEQ ID NO:131. In some embodiments, the T cells comprises a nucleic acid having the nucleotide sequence of SEQ ID NO:131. In some embodiments, the endogenous genes encoding β2M and α subunit of TCR are inactivated in the T cells. In some embodiments, the endogenous genes encoding β2M and α subunit of TCR are knocked out in the T cells.

In some embodiments, the T cells comprises a nucleic acid that encodes an amino acid sequence that is at least 85% identical to SEQ ID NO:134. In some embodiments, the T cells comprises a nucleic acid that encodes an amino acid sequence that is at least 90% identical to SEQ ID NO:134. In some embodiments, the T cells comprises a nucleic acid that encodes an amino acid sequence that is at least 95% identical to SEQ ID NO:134. In some embodiments, the T cells comprises a nucleic acid that encodes an amino acid sequence that is at least 98% identical to SEQ ID NO:134. In some embodiments, the T cells comprises a nucleic acid that encodes an amino acid sequence that is at least 99% identical to SEQ ID NO:134. In some embodiments, the T cells comprises a nucleic acid that encodes the amino acid sequence of SEQ ID NO:134. In some embodiments, the T cells comprises a nucleic acid that is at least 85%, at least 90%, at least 95%, at least 98%, or at least 99% identical to SEQ ID NO:135. In some embodiments, the T cells comprises a nucleic acid having the nucleotide sequence of SEQ ID NO:135. In some embodiments, the endogenous genes encoding β2M and α subunit of TCR are inactivated in the T cells. In some embodiments, the endogenous genes encoding β2M and α subunit of TCR are knocked out in the T cells.

In some embodiments, the T cells comprises a nucleic acid that encodes an amino acid sequence that is at least 85% identical to SEQ ID NO:138. In some embodiments, the T cells comprises a nucleic acid that encodes an amino acid sequence that is at least 90% identical to SEQ ID NO:138. In some embodiments, the T cells comprises a nucleic acid that encodes an amino acid sequence that is at least 95% identical to SEQ ID NO:138. In some embodiments, the T cells comprises a nucleic acid that encodes an amino acid sequence that is at least 98% identical to SEQ ID NO:138. In some embodiments, the T cells comprises a nucleic acid that encodes an amino acid sequence that is at least 99% identical to SEQ ID NO:138. In some embodiments, the T cells comprises a nucleic acid that encodes the amino acid sequence of SEQ ID NO:138. In some embodiments, the T cells comprises a nucleic acid that is at least 85%, at least 90%, at least 95%, at least 98%, or at least 99% identical to SEQ ID NO:139. In some embodiments, the T cells comprises a nucleic acid having the nucleotide sequence of SEQ ID NO:139. In some embodiments, the endogenous genes encoding β2M and α subunit of TCR are inactivated in the T cells. In some embodiments, the endogenous genes encoding β2M and α subunit of TCR are knocked out in the T cells.

### 6.6 Pharmaceutical Compositions

Provided herein are also pharmaceutical compositions comprising the cells disclosed herein. The pharmaceutical composition comprises an effective amount of a cell disclosed herein and a pharmaceutically acceptable carrier. The cells disclosed herein and compositions comprising the cells can be conveniently provided in sterile liquid preparations, for example, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells disclosed herein in a suitable amount of the appropriate solvent with various amounts of the other ingredients, as desired. Such compositions can include a pharmaceutically acceptable carrier, diluent, or excipient such as sterile water, physiological saline, glucose, dextrose, or the like, that are suitable for use with a cell composition and for administration to a subject such as a human. Suitable buffers for providing a cell composition are well known in the art. Any vehicle, diluent, or additive used is compatible with preserving the integrity and viability of the cells disclosed herein.

The compositions will generally be isotonic, that is, they have the same osmotic pressure as blood and lacrimal fluid. The desired isotonicity of the cell compositions provided herein can be accomplished using sodium chloride, or other pharmaceutically acceptable agents such as dextrose, boric acid, sodium tartrate, or other inorganic or organic solutes. Sodium chloride is preferred particularly for buffers containing sodium ions. One particularly useful buffer is saline, for example, normal saline. Those skilled in the art will recognize that the components of the compositions should be selected to be chemically inert and will not affect the viability or efficacy of the cells disclosed herein and will be compatible for administration to a subject, such as a human. The skilled artisan can readily determine the amount of cells and optional additives, vehicles, and/or carrier in compositions to be administered in methods of the invention.

The cells disclosed herein can be administered in any physiologically acceptable vehicle. Suitable doses for administration are described herein. A cell population comprising cells disclosed herein can comprise a purified population of cells. Those skilled in the art can readily determine the percentage of cells in a cell population using various well-known methods, as described herein. The ranges of purity in cell populations comprising genetically modified cells provided herein can be from about 20% to about 25%, from about 25% to about 30%, from about 30% to about 35%, from about 35% to about 40%, from about 40% to about 45%, from about 45% to about 50%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. In some embodiments, the ranges of purity in cell populations comprising genetically modified cells provided herein can be from about 20% to about 30%, from about 20% to about 50%, from about 20% to about 80%, from about 20% to about 100%, from about 50% to about 80%, or from about 50% to about 100%. Dosages can be readily adjusted by those skilled in the art; for example, a decrease in purity may require an increase in dosage.

Provided herein are also kits for preparation of cells disclosed herein. In one embodiment, the kit comprises one or more vectors for generating a genetically engineered cell, such as a T cell, that expresses a fusion protein disclosed herein for allogeneic transplant. The kits can be used to generate genetically engineered cells from non-autologous cells to be administered to a compatible subject. In another embodiment, the kits can comprise cells disclosed herein, for example, non-autologous cells, for administration to a subject. In specific embodiments, the kits comprise the cells disclosed herein in one or more containers.

### 6.7 Methods of Production

Provided herein are methods of genetically engineering a cell for an allogeneic transplant, comprising transducing the cell with a nucleic acid disclosed herein. Provided herein are also methods of genetically engineering a cell for an allogeneic transplant, comprising transducing the cell with a vector comprising a nucleic acid disclosed herein. In some embodiments, the nucleic acid encodes a fusion protein disclosed herein. In some embodiments, methods provided herein comprise transducing a cell with a nucleic acid encoding a fusion protein, wherein the fusion protein comprises a presenting peptide and a β2M peptide covalently linked via a linker.

In some embodiments, provided herein are methods of genetically engineering a cell for an allogeneic transplant, comprising transducing the cell with a nucleic acid that encodes a fusion protein comprising a presenting peptide covalently linked to a β2M peptide via a linker, wherein the fusion protein binds a MHC heavy chain to form an MHC complex that binds an inhibitory receptor of an immune cell to inhibit the immune cell. In some embodiments, provided herein are methods of genetically engineering a cell for an allogeneic transplant, comprising transducing the cell with a nucleic acid that encodes a fusion protein comprising an HLA-E-restricted presenting peptide covalently linked to a β2M peptide via a linker. In some embodiments, provided herein are methods of genetically engineering a cell for an allogeneic transplant, comprising transducing the cell with a nucleic acid that encodes a fusion protein comprising a presenting peptide covalently linked to a β2M peptide via a linker, wherein the presenting peptide is a signal peptide of a Class I MHC molecule, or a fragment thereof. In some embodiments, the fusion protein does not have an MHC heavy chain. In some embodiments, the fusion protein does not have an HLA-E heavy chain. In some embodiments, the fusion protein does not have an HLA-A heavy chain, an HLA-B heavy chain, an HLA-C heavy chain, an HLA-E heavy chain, an HLA-F heavy chain, or an HLA-G heavy chain. In some embodiments, the fusion protein has less than 500 amino acids, less than 400 amino acids, less than 300 amino acids, or less than 200 amino acids. In some embodiments, the fusion protein has about 100 to about 300 amino acids, about 100 to about 200 amino acids, about 120 to about 180 amino acids, about 120 to about 160 amino acids, or about 140 to about 160 amino acids. Various embodiments of the fusion proteins encoded by the nucleic acids provided herein are provided in Section 6.2 above.

The cells disclosed herein (e.g. stem cells or immune cells) can be subjected to conditions that favor maintenance or expansion of cells as well known in the art. (De Libero, T Cell Protocols, Vol. 514 of Methods in Molecular Biology, Humana Press, Totowa NJ (2009); Parente-Pereira et al., J. Biol. Methods 1(2) e7 (doi 10.14440/jbm.2014.30) (2014); Movassagh et al., Hum. Gene Ther. 11:1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1999); Quinn et al., Hum. Gene Ther. 9:1457-1467 (1998); see also commercially available methods such as Dynabeads™ human T cell activator products, Thermo Fisher Scientific, Waltham, MA)). The cells disclosed herein (e.g. stem cells or immune cells) can optionally be expanded prior to or after ex vivo genetic engineering. Expansion of the cells is particularly useful to increase the number of cells for administration to a subject. Such methods for expansion of cells are well known in the art (see e.g. Kaiser et al., Cancer Gene Therapy 22:72-78 (2015); Wolfi et al., Nat. Protocols 9:950-966 (2014)). Furthermore, the cells can optionally be cryopreserved after isolation and/or genetic engineering, and/or expansion of genetically engineered cells (see Kaiser et al., supra, 2015)). Methods for cyropreserving cells are well known in the art (see, for example, Freshney, Culture of Animal Cells: A Manual of Basic Techniques, 4th ed., Wiley-Liss, New York (2000); Harrison and Rae, General Techniques of Cell Culture, Cambridge University Press (1997)).

With respect to generating cells recombinantly expressing a fusion protein disclosed herein, one or more nucleic acids encoding the fusion protein is introduced into the target cell using a suitable expression vector. The target cells (e.g. stem cells or immune cells) are transduced with one or more nucleic acids encoding a fusion protein, or a synthetic receptor and a fusion protein. In the case of expressing both a synthetic receptor and fusion protein, the synthetic receptor and fusion protein encoding nucleic acids can be on separate vectors or on the same vector, as desired. For example, a nucleic acid encoding a synthetic receptor or a fusion protein disclosed herein can be cloned into a suitable vector, such as a retroviral vector, and introduced into the target cell using well known molecular biology techniques (see Ausubel et al., Current Protocols in Molecular Biology, John Wiley and Sons, Baltimore, MD (1999)). Any vector suitable for expression in a cell of the invention, particularly a human immune cell or a stem cell, can be used. The vectors contain suitable expression elements such as promoters that provide for expression of the encoded nucleic acids in the target cell. In the case of a retroviral vector, cells can optionally be activated to increase transduction efficiency (see Parente-Pereira et al., J. Biol. Methods 1(2) e7 (doi 10.14440/jbm.2014.30) (2014); Movassagh et al., Hum. Gene Ther. 11:1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1998); Quinn et al., Hum. Gene Ther. 9:1457-1467 (1998); see also commercially available methods such as Dynabeads™ human T cell activator products, Thermo Fisher Scientific, Waltham, MA).

In one embodiment, the vector is a retroviral vector, for example, a gamma retroviral or lentiviral vector, which is employed for the introduction of a fusion protein and/or synthetic receptor into the target cell. For genetic modification of the cells to express a fusion protein and/or synthetic receptor, a retroviral vector is generally employed for transduction. However, it is understood that any suitable viral vector or non-viral delivery system can be used. Combinations of a retroviral vector and an appropriate packaging line are also suitable, where the capsid proteins will be functional for infecting human cells. Various amphotropic virus-producing cell lines are known, including, but not limited to, PA12 (Miller et al., Mol. Cell. Biol. 5:431-437 (1985)); PA317 (Miller et al., Mol. Cell. Biol. 6:2895-2902(1986)); and CRIP (Danos et al., Proc. Natl. Acad. Sci. USA 85:6460-6464 (1988)). Non-amphotropic particles are suitable too, for example, particles pseudotyped with VSVG, RD 114 or GALV envelope and any other known in the art (Relander et al., Mol. Therap. 11:452-459 (2005)). Possible methods of transduction also include direct co-culture of the cells with producer cells (for example, Bregni et al., Blood 80:1418-1422 (1992)), or culturing with viral supernatant alone or concentrated vector stocks with or without appropriate growth factors and polycations (see, for example, Xu et al., Exp. Hemat. 22:223-230 (1994); Hughes, et al. J. Clin. Invest. 89:1817-1824 (1992)).

Generally, the chosen vector exhibits high efficiency of infection and stable integration and expression (see, for example, Cayouette et al., Human Gene Therapy 8:423-430 (1997); Kido et al., Current Eye Research 15:833-844 (1996); Bloomer et al., J. Virol. 71:6641-6649 (1997); Naldini et al., Science 272:263 267 (1996); and Miyoshi et al., Proc. Natl. Acad. Sci. U.S.A. 94:10319-10323 (1997)). Other viral vectors that can be used include, for example, adenoviral, lentiviral, and adeno-associated viral vectors, vaccinia virus, a bovine papilloma virus derived vector, or a herpes virus, such as Epstein-Barr Virus (see, for example, Miller, Hum. Gene Ther. 1(1):5-14 (1990); Friedman, Science 244:1275-1281 (1989); Eglitis et al., BioTechniques 6:608-614 (1988); Tolstoshev et al., Current Opin. Biotechnol. 1:55-61 (1990); Sharp, Lancet 337:1277-1278 (1991); Cornetta et al., Prog. Nucleic Acid Res. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J Med. 323:370 (1990); Anderson et al., U.S. Pat. No. 5,399,346).

Particularly useful vectors for expressing a fusion protein disclosed herein and/or synthetic receptor include vectors that have been used in human gene therapy. In one non-limiting embodiment, a vector is a retroviral vector. The use of retroviral vectors for expression in T cells or other immune cells, including engineered T cells, has been described (see Scholler et al., Sci. Transl. Med. 4:132-153 (2012; Parente-Pereira et al., J. Biol. Methods 1(2):e7 (1-9)(2014); Lamers et al., Blood 117(1):72-82 (2011); Reviere et al., Proc. Natl. Acad. Sci. USA 92:6733-6737 (1995)). In one embodiment, the vector is an SGF retroviral vector such as an SGF γ-retroviral vector, which is Moloney murine leukemia-based retroviral vector. SGF vectors have been described previously (see, for example, Wang et al., Gene Therapy 15:1454-1459 (2008)).

The vectors used herein employ suitable promoters for expression in a particular host cell. The promoter can be an inducible promoter or a constitutive promoter. In a particular embodiment, the promoter of an expression vector provides expression in a stem cell, such as a hematopoietic stem cell. In a particular embodiment, the promoter of an expression vector provides expression in an immune cell, such as a T cell. Non-viral vectors can be used as well, so long as the vector contains suitable expression elements for expression in the target cell. Some vectors, such as retroviral vectors, can integrate into the host genome. If desired, targeted integration can be implemented using technologies such as a nuclease, transcription activator-like effector nucleases (TALENs), Zinc-finger nucleases (ZFNs), and/or clustered regularly interspaced short palindromic repeats (CRISPRs), homologous recombination, non-homologous end joining, microhomology-mediated end joining, homology-mediated end joining and the like (Gersbach et al., Nucl. Acids Res. 39:7868-7878 (2011); Vasileva, et al. Cell Death Dis. 6:e1831. (Jul. 23 2015); Sontheimer, Hum. Gene Ther. 26(7):413-424 (2015); Yao et al. Cell Research volume 27, pages 801-814(2017)).

The vectors and constructs can optionally be designed to include a reporter. For example, the vector can be designed to express a reporter protein, which can be useful to identify cells comprising the vector or nucleic acids provided on the vector, such as nucleic acids that have integrated into the host chromosome. In one embodiment, the reporter can be expressed as a bicistronic or multicistronic expression construct with the fusion protein or synthetic receptor. Exemplary reporter proteins include, but are not limited to, fluorescent proteins, such as mCherry, green fluorescent protein (GFP), blue fluorescent protein, for example, EBFP, EBFP2, Azurite, and mKalama1, cyan fluorescent protein, for example, ECFP, Cerulean, and CyPet, and yellow fluorescent protein, for example, YFP, Citrine, Venus, and YPet.

Assays can be used to determine the transduction efficiency of a fusion protein disclosed herein or a synthetic receptor using routine molecular biology techniques. If a marker has been included in the construct, such as a fluorescent protein, gene transfer efficiency can be monitored by FACS analysis to quantify the fraction of transduced (for example, GFP⁺) immune cells, such as T cells, and/or by quantitative PCR. Using a well-established cocultivation system (Gade et al., Cancer Res. 65:9080-9088 (2005); Gong et al., Neoplasia 1:123-127 (1999); Latouche et al., Nat. Biotechnol. 18:405-409 (2000)) it can be determined whether fibroblast AAPCs expressing cancer antigen (vs. controls) direct cytokine release from transduced immune cells, such as T cells, expressing a synthetic receptor (e.g. CAR) (cell supernatant LUMINEX (Austin TX) assay for IL-2, IL-4, IL-10, IFN-γ, TNF-α, and GM-CSF), T cell proliferation (by carboxyfluorescein succinimidyl ester (CFSE) labeling), and T cell survival (by Annexin V staining). The influence of CD80 and/or 4-1BBL on T cell survival, proliferation, and efficacy can be evaluated. T cells can be exposed to repeated stimulation by cancer antigen positive target cells, and it can be determined whether T cell proliferation and cytokine response remain similar or diminished with repeated stimulation. The cancer antigen CAR constructs can be compared side by side under equivalent assay conditions. Cytotoxicity assays with multiple E:T ratios can be conducted using chromium-release assays.

In addition to providing a nucleic acid encoding a fusion protein disclosed herein and/or a synthetic receptor in a vector for expression in an immune cell, a nucleic acid encoding the fusion protein and/or synthetic receptor can also be provided in other types of vectors more suitable for genetic manipulation, such as for expression of various constructs in a bacterial cell such as E. coli. Such vectors can be any of the well-known expression vectors, including commercially available expression vectors (see in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

Provided herein are also methods of genetically engineering a cell for an allogeneic transplant that include inactivating a gene. In some embodiments, methods provided herein include inactivating at least one gene encoding a Class I MHC molecule or an MHC-like molecule in the immune cell. In some embodiments, the Class I MHC molecule is selected from the group consisting of heavy chains of HLA-A, HLA-B, HLA-C, HLA-E, HLA-F, and HLA-G. In some embodiments, methods provided herein include inactivating the gene encoding β2M. In some embodiments, methods provided herein include inactivating a gene encoding an MHC-like molecule selected from the group consisting of CD1 heavy chain, MR1 heavy chain, FcRN heavy chain and UL18.

Gene inactivation can be done using any methods disclosed herein or otherwise known in the art. In some embodiments, the gene is inactivated DNA cleavage and repair, base editing, RNA interference, or RNA editing. In some embodiments, the gene is inactivated by DNA cleavage using a rare endonuclease selected from the group consisting of a RNA-directed endonuclease, a TAL nuclease, a homing endonuclease, a zinc-finger nuclease, and a Mega-TAL nuclease. In some embodiments, the gene is inactivated by DNA cleavage and repair. In some embodiments, the DNA cleavage and repair is performed by using a CRISPR-Cas system. The CRISPR-Cas system is a CRISPR-Cas9 system.

### Base Editing

In some embodiments, methods provided herein comprise inactivating a gene by base editing. Accurate site-directed change and transformation among adenine (A), guanine (G), cytosine (C), thymine (T), or uracil (U) at a certain specific site in a nucleic acid (DNA or RNA) result in the base at the specific site changing from one base to another base.

### Prime Editing

In some embodiments, methods provided herein comprise inactivating a gene by prime editing. It's a “search-and-replace” genome editing technique that mediates targeted insertions, deletions, and all possible base-to-base conversions. And, it can combine different types of edits with one another. For example, a primer editing process can include the following steps: first, an engineered prime editing guide RNA (pegRNA) that both specifies the target site and contains the desired edit(s) engages the prime editor protein. This primer editor protein consists of a Cas9 nickase fused to a reverse transcriptase. The Cas9 nickase part of the protein is guided to the DNA target site by the pegRNA. After nicking by Cas9, the reverse transcriptase domain uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Lastly, the editor guides resolution of the heteroduplex to favor copying the edit onto the unedited strand, completing the process (Anzalone et al. Nature (2019) doi:10.1038/s41586-019-1711-4).

### RNA Interference

In some embodiments, methods provided herein comprise inactivating a gene by RNA interference. RNA interference (RNAi) can be adopted for reducing or silencing the expression of a target gene. In an embodiment, for example, the degradation of a target gene mRNA (or its stability is interfered) is specifically induced or the translation efficiency of the target gene mRNA is inhibited by expressing a double-stranded RNA or an antisense RNA homologous to the target gene mRNA.

### DNA Cleavage and Repair

In some embodiments, methods provided herein comprise inactivating a gene by DNA cleavage and repair. DNA cleavage refers to a method that one or two DNA strands at a double-stranded DNA target sequence site break. DNA cleavage can be done, for example, by using a ribonuclease, an FokI endonuclease, a Cas protein and the like. After the DNA is cleaved, the cell repairs the cleavage site, for example, by a non-homologous end joining (NHEJ) or homologous recombination (HR). The sequence near the cleavage site can be changed, and errors such as insertion and deletion mutations may occur. If the cleavage site is in an exon region or a regulatory region of a gene, the expression of the gene can be reduced and eliminated.

### Zinc-Finger Nuclease (ZFN)

In some embodiments, methods provided herein comprise inactivating a gene by using Zinc-finger nuclease (ZFN). A zinc-finger nuclease consists of a DNA recognition domain and a non-specific endonuclease. The DNA recognition domain consists of a series of Cys2-His2 zinc-finger proteins linked in series, and each zinc-finger unit includes about 30 amino acids for specifically binding to DNA. The non-specific endonuclease is a FokI endonuclease which forms a dimer to cleave the DNA.

### TAL Nuclease (TALEN)

In some embodiments, methods provided herein comprise inactivating a gene by using TALEN. TALEN is a transcription activator-like effector nuclease. The TALE protein is a core component of a DNA binding domain, and generally consists of a plurality of basic repeat units linked in series. The designed and combined series of units can specifically recognize a DNA sequence, and cleave a specific DNA sequence by coupling the FokI endonuclease.

### CRISPR/Cas System

In some embodiments, methods provided herein comprise inactivating a gene by using the CRISPR/Cas system. A CRISPR/Cas system is a nuclease system consisting of clustered regularly interspaced short palindromic repeats (CRISPR) and CRISPR binding proteins (i.e., Cas proteins), which can cleave nearly all genomic sequences adjacent to protospacer-adjacent motifs (PAM) in eukaryocytes (Cong et al. Science 2013. 339: 819-823). The “CRISPR/Cas system” is used to refer collectively to transcripts involving CRISPR-related (“Cas”) genes, as well as other elements involving the expression thereof or directing the activity thereof, including sequences encoding a Cas gene, tracr (trans-activated CRISPR) sequences (for example, tracrRNA or active partial tracrRNA), tracr pairing sequences (in the background of an endogenous CRISPR system, cover “direct repeats” and processed partial direct repeats), guide sequences, or other sequences from the CRISPR locus and transcripts. In general, the CRISPR system is characterized as an element that facilitates the formation of a CRISPR complex at a site of a target sequence (also called a protospacer in the endogenous CRISPR system).

The formation of the CRISPR complex (comprising a guide sequence that is hybridized to the target sequence and complexes with one or more Cas proteins) results in the cleavage of one chain or two chains in or near the target sequence (for example, within 1, 2, 3, 4, 5, 6, 7, 8, 9, 10, 20, 50, or more base pairs). The tracr sequence (which may comprise or consist of all or part of a wild-type tracr sequence (for example, about or more than about 20, 26, 32, 45, 48, 54, 63, 67, 85 or more nucleotides of the wild-type tracr sequence)) may also form part of the CRISPR complex, for example, by hybridizing along at least a part of the tracr sequence to all or part of the tracr pairing sequence operably linked to the guide sequence. In some embodiments, the tracr sequence is sufficiently complementary to a tracr pairing sequence to hybridize and participate in the formation of a CRISPR complex. In some embodiments, the tracr sequence has at least 50%, 60%, 70%, 80%, 90%, 95%, or 99% sequence complementarity along the length of the tracr pairing sequence when performing an optimal comparison. In some embodiments, one or more vectors that drive expression of one or more elements of the CRISPR system are introduced into a host cell such that the expression of the elements of the CRISPR system directs the formation of the CRISPR complex at one or more target sites.

In general, the tracr pairing sequence has sufficient complementarity with the tracr sequence to facilitate formation of the CRISPR complex at the target sequence, wherein the CRISPR complex comprises a tracr pairing sequence that is hybridized to the tracr sequence. Generally, the degree of complementarity is in terms of an optimal alignment of the tracr pairing sequence and the tracr sequence along the length of the shorter of the two sequences. The optimal alignment can be determined by any suitable alignment algorithm, and the effects of a secondary structure can be further taken into account, for example, the self-complementarity within the tracr sequence or tracr pairing sequence. When performing the optimal alignment, the degree of complementarity between the tracr sequence and the tracr pairing sequence along the length of the shorter of the two is about or more than about 25%, 30%, 40%, 50%, 60%, 70%, 80%, 90%, 95%, 97.5%, 99%, or higher. The tracr sequence has about or more than about 5, 6, 7, 8, 9, 10, 11, 12, 13, 14, 15, 16, 17, 18, 19, 20, 25, 30, 40, 50 or more nucleotides in sequence.

Unrestricted examples of the Cas protein include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 homologues, or modified forms thereof. In some embodiments, the Cas protein is a Cas9 protein.

Cas9, also known as Csn1, is a giant protein involved in both crRNA biosynthesis and the destruction of invading DNA. Cas9 has been described in different bacterial species such as S. thermophiles, Listeria innocua (Gasiunas, Barrangou et al. 2012; Jinek, Chylinski et al. 2012) and S. pyogenes (Deltcheva, Chylinski et al. 2011). The giant Cas9 protein (more than 1200 amino acids) contains two predicted nuclease domains, namely, an HNH (McrA-like) nuclease domain located in the middle of the protein and a split RuvC-like nuclease domain (RNAase H-fold) (Makarova, Grishin et al. (2006)). A Cas9 variant may be a Cas9 endonuclease that is not naturally found in nature and is obtained by protein engineering or by random mutagenesis. For example, the Cas9 variant according to the present invention can be obtained by mutations such as deletion, insertion or substitution of at least one residue in the amino acid sequence of the S. pyogenes Cas9 endonuclease (COG3513). In some embodiments, the Cas9 protein is Streptococcus pneumoniae, S. pyogenes, or S. thermophiles Cas9, and may include mutated Cas9 derived from these organisms, or variants linking other amino acid sequences to Cas9, for example, the variant linking the FokI enzyme to Cas9. These Cas9 are known; for example, the amino acid sequence of the Streptococcus pyogenes Cas9 protein can be found in the SwissProt database under the accession number Q99ZW2, the amino acid sequence of the Neisseria meningitides Cas9 protein can be found in the UniProt database under the number A1IQ68, the amino acid sequence of the S. thermophiles Cas9 protein can be found in the UniProt database under the number Q03LF7, and the amino acid sequence of the Staphylococcus aureus Cas9 protein can be found in the UniProt database under the number J7RUA5.

Combinations and permutations of various methods described herein or otherwise known in the art are expressly contemplated to prepare the genetically engineered cells disclosed herein.

### 6.8 Methods of Treatment

The genetically engineered cells provided herein can be used in allogeneic transplant, and are less likely to be rejected by the recipient's immune system, at least because the fusion proteins expressed on the cells can form MHC complexes that engage the inhibitory receptors on the recipient's immune cells to prevent their activation against the allogeneic transplant.

In some embodiments, provided herein are uses of a genetically engineered cell in an allogeneic transplant, wherein the cell expresses a fusion protein comprising a presenting peptide and a β2M peptide covalently linked via a linker. Provided herein are also methods of providing an allogeneic cell transplant to a subject in need thereof, comprising administering a therapeutically effective amount of a genetically engineered cell to the subject, wherein the cell expresses a fusion protein comprising a presenting peptide and a β2M peptide covalently linked via a linker. The genetically engineered cell can be any genetically engineered cell disclosed herein in section 6.5. The fusion protein can be any fusion protein disclosed herein in section 6.2.

In some embodiments, provided herein are uses of a genetically engineered cell in an allogeneic transplant, wherein the cell comprises a nucleic acid disclosed herein. Provided herein are also methods of providing an allogeneic cell transplant in a subject in need thereof, comprising administering a therapeutically effective amount of a genetically engineered cell to the subject, wherein the cell comprises a nucleic acid disclosed herein.

In some embodiments, the subject receiving the treatment is a human. In some embodiments, the subject is a human in need of an allogeneic transplant.

The fusion proteins, nucleic acids, cells, and the methods of their production are disclosed in detail in sections above. For illustrative purposes, provided herein are uses of a genetically engineered cell in allogeneic transplant, wherein the cell comprises a nucleic acid encoding a fusion protein comprising an HLA-E-restricted presenting peptide covalently linked to human β2M via a (G4S)3 linker. The fusion protein can bind an endogenously expressed HLA-E heavy chain to form an HLA-E complex on the surface of the genetically engineered cell, which can engage an inhibitory receptor (e.g. NKG2A) on an immune cell (e.g. NK cell) to inhibit its potentially cytotoxic activity against the genetically engineered cells.

As such, provided herein are methods of administering a therapeutically effective amount of the genetically engineered cells disclosed herein to a subject in need of such cells as an allogeneic transplant. Provided herein are also uses of the genetically engineered cells disclosed herein for preparing an allogeneic transplant. An allogeneic transplant comprising the cells provided herein is less likely to be rejected by the recipient, at least because the fusion protein expressed in the cells provides a mechanism for inhibiting a potential allogeneic immune response. In some embodiments, provided herein are methods of providing an allogeneic transplant to a subject in need thereof by administering a therapeutically effective amount of cells provided herein to the subject. In some embodiments, provided herein are methods of reducing the likelihood that cells administered to a subject trigger an allogeneic immune response in the subject. In some embodiments, provided herein are methods of reducing an allogeneic response in a subject. In some embodiments, methods provided herein reduce the occurrence of allograft rejection.

The methods of administering cells disclosed herein can be adapted for any purpose in which administering such cells is desirable. In some embodiments, the subject in need of administration of cells is suffering from a disorder. For example, the subject can be suffering from a disorder in which the particular cells are decreased in function or number, and it is therefore desirable to administer functional cells obtained from a healthy or normal individual in which the particular cells are functioning properly and to administer an adequate number of those healthy cells to the individual to restore the function provided by those cells (e.g., hormone producing cells which have decreased in cell number or function, immune cells winch have decreased in cell number or function, etc.). In such instances, the healthy cells can be engineered as disclosed herein to decrease the likelihood of host rejection of the healthy cells. In some embodiments, the disorder is a genetic disorder. In some embodiments, the disorder is an infection. In some embodiments, the disorder is HIV infection or AIDS. In some embodiments, the disorder is cancer.

Methods disclosed herein can be particularly helpful in regenerative medicine. For example, patients in need of a stem cell transplant can benefit from the genetically engineered cells disclosed herein. In some embodiments, provided herein are methods of providing an allogeneic stem cell to a subject in need thereof by administering a therapeutically effective amount of the stem cells provided herein to the subject. Provided herein are also uses of the genetically engineered stem cells disclosed herein for preparing a stem cell transplant. In some embodiments, the genetically engineered stem cells are differentiated into the desired cell type before being administered to a subject in need thereof. For illustrative purposes, people who can benefit from these cell therapies include those with spinal cord injuries, type 1 diabetes, Parkinson's disease, amyotrophic lateral sclerosis, Alzheimer's disease, heart disease, stroke, burns, cancer and osteoarthritis. In some embodiments, provided herein are methods of treating a subject that can be benefit from an allogenic cell therapy by administering a therapeutically effective amount of the cells provided herein to the subject. In some embodiments, the subject can have spinal cord injuries, type 1 diabetes, Parkinson's disease, amyotrophic lateral sclerosis, Alzheimer's disease, heart disease, stroke, burns, cancer or osteoarthritis.

Provided herein are methods of using the cells, for example, T cells, expressing the fusion protein described herein in allogeneic transplant. In some embodiments, the genetically engineered cells can be expanded before being administered to a subject in need thereof. The cells are administered as a population of cells expressing the fusion protein described herein. Optionally, the cells to be administered can be purified or enriched. In some embodiments, the subject is in need of an organ transplantation, and the allogeneic organ transplant can be developed using a genetically engineered stem cell disclosed herein. As such, provided herein are also methods of providing an allogenic organ transplant by culturing the genetically engineered stem cells disclosed herein under appropriate conditions that allow the development of the organ.

In some embodiments, provided herein are methods of providing hematopoietic stem and/or progenitor cells to a subject in need thereof by administering a therapeutically effective amount of the genetically engineered cells provided herein to the subject in need thereof, wherein the cells are hematopoietic stem and/or progenitor cells. The subject might be in need of a bone marrow transplant, and provided herein are also uses of the genetically engineered cells disclosed herein for preparing a bone marrow transplant. The genetically engineered hematopoietic stem and/or progenitor cells can be CD34+ mobilized peripheral blood cells, CD34+ cord blood cells, or CD34+ bone marrow cells.

In some embodiments, provided herein are methods of treating cancer by administering a therapeutically effective amount of the cells provided herein to the subject in need thereof. Provided herein are also uses of the genetically engineered cells disclosed herein in cancer treatment. Provided herein are also uses of the genetically engineered cells disclosed herein for the preparation of a medicament for the treatment of cancer. The cell can be an immune cell. The cell can be a leukocyte, including, for example, myeloid cells, lymphoid cells, granulocytes (such as neutrophils, eosinophils, basophils), lymphocytes (such as T cells, B cells, NK cells, NKT cells), plasma cells, dendritic cells, monocytes, or cells differentiated therefrom such as a macrophage. In some embodiments, provided herein are methods of treating cancer by administering a therapeutically effective amount of the genetically engineered immune cells provided herein to the subject in need thereof. In some embodiments, provided herein are methods of treating cancer by administering a therapeutically effective amount of the genetically engineered T cells provided herein to the subject in need thereof. In some embodiments, provided herein are methods of treating cancer by administering a therapeutically effective amount of the genetically engineered NK cells provided herein to the subject in need thereof. In some embodiments, provided herein are methods of treating cancer by administering a therapeutically effective amount of the genetically engineered macrophages provided herein to the subject in need thereof.

In some embodiments, provided herein are also uses of the genetically engineered immune cells disclosed herein in preparation of a medicament for cancer treatment. In another embodiment, the methods can include administering a cancer-antigen specific immune cell to a subject in need thereof, wherein the cell recombinantly expresses a fusion protein disclosed herein and a synthetic receptor comprising an antigen binding domain that specifically binds the cancer antigen. The cancer antigen can be any cancer antigen disclosed herein or otherwise known in the art. In some embodiments, the cancer antigen is selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1. In some embodiments, the synthetic receptor is a CAR, a TCR, a TRuC, a TAC, an AbTCR, or a chimeric CD3 receptor.

In some embodiments, provided herein are also uses of the genetically engineered T cells disclosed herein in preparation of a medicament for cancer treatment, wherein the genetically engineered T cells express a synthetic receptor. In some embodiments, the genetically engineered T cells express a CAR. For example, provided herein are universal CAR-Ts that can be used in cancer treatment. In some embodiments, the genetically engineered T cells express a TCR. In some embodiments, the genetically engineered T cells express a TRuC. In some embodiments, the genetically engineered T cells express a TAC. In some embodiments, the genetically engineered T cells express an AbTCR. In some embodiments, the genetically engineered T cells express a chimeric CD3 receptor.

In some embodiments, immune cells provided herein can be used in allogeneic transplantation. The immune cell can be a leukocyte, including, for example, myeloid cells, lymphoid cells, granulocytes (such as neutrophils, eosinophils, basophils), lymphocytes (such as T cells, B cells, NK cells, NKT cells), plasma cells, dendritic cells, monocytes, or cells differentiated therefrom such as a macrophage. In some embodiments, provided herein are methods of treating cancer in a subject by administering a therapeutically effective amount of the T cells provided herein to the subject in need thereof. In some embodiments, provided herein are methods of treating cancer in a subject by administering a therapeutically effective amount of the CAR-T cells provided herein to the subject in need thereof. Provided herein are also uses of the genetically engineered T cells disclosed herein in cancer treatment. Provided herein are also uses of the genetically engineered T cells disclosed herein in preparation of a medicament for cancer treatment.

For example, the methods can be used to treat cancer or reduce tumor burden in a subject. In one embodiment, the methods provided herein are used to treat cancer. It is understood that a method of treating cancer can include any effect that ameliorates a sign or symptom associated with cancer. Such signs or symptoms include, but are not limited to, reducing tumor burden, including inhibiting growth of a tumor, slowing the growth rate of a tumor, reducing the size of a tumor, reducing the number of tumors, eliminating a tumor, all of which can be measured using routine tumor imaging techniques well known in the art. Other signs or symptoms associated with cancer include, but are not limited to, fatigue, pain, weight loss, and other signs or symptoms associated with various cancers. In one non-limiting example, the methods of the invention can reduce tumor burden. Thus, administration of the cells of the invention can reduce the number of tumor cells, reduce tumor size, and/or eradicate the tumor in the subject.

In the methods provided herein, the immune cells are administered to a subject in need of cancer treatment. The subject can be a mammal, in particular a human. Preferably, the subject is a human. Suitable human subjects for therapy include those with “advanced disease” or “high tumor burden” who bear a clinically measurable tumor. A clinically measurable tumor is one that can be detected on the basis of tumor mass, for example, by palpation, CAT scan, sonogram, mammogram, X-ray, and the like. Positive biochemical or histopathologic markers can also be used to identify this population. A pharmaceutical composition comprising a cell provided herein is administered to a subject to elicit an anti-cancer response, with the objective of palliating the subject's condition. Reduction in tumor mass of a subject having a tumor can occur, but any clinical improvement constitutes a benefit. Clinical improvement comprises decreased risk or rate of progression or reduction in pathological consequences of the tumor.

Another group of suitable subjects can be a subject who has a history of cancer, but has been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for different types of cancers. Features typical of high-risk subgroups are those in which the tumor has invaded neighboring tissues, or who show involvement of lymph nodes. Optionally, a cell provided herein can be administered for treatment prophylactically to prevent the occurrence of cancer in a subject suspected of having a predisposition to a cancer, for example, based on family history and/or genetic testing.

The subject can have an advanced form of disease, in which case the treatment objective can include mitigation or reversal of disease progression, and/or amelioration of side effects. The subjects can have a history of the condition, for which they have already been treated, in which case the therapeutic objective can be to decrease or delay the risk of recurrence. Additionally, refractory or recurrent malignancies can be treated using the cells provided herein.

The cells provided herein are administered to a subject, such as a human subject, in need of cancer treatment. The cancer can involve a solid tumor or a blood cancer not involving a solid tumor. Cancers to be treated using the cells of the invention comprise cancers typically responsive to immunotherapy. Exemplary types of cancers include, but are not limited to, carcinomas, sarcoma, leukemia, lymphoma, multiple myeloma, melanoma, brain and spinal cord tumors, germ cell tumors, neuroendocrine tumors, carcinoid tumors, and the like. The cancer can be a solid tumor or a blood cancer that does not form a solid tumor. In the case of a solid tumor, the tumor can be a primary tumor or a metastatic tumor.

Examples of other neoplasias or cancers that can be treated using the methods provided herein include bone cancer, intestinal cancer, liver cancer, skin cancer, cancer of the head or neck, melanoma (cutaneous or intraocular malignant melanoma), renal cancer (for example, clear cell carcinoma), throat cancer, prostate cancer (for example, hormone refractory prostate adenocarcinoma), blood cancers (for example, leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (for example, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease, Waldenstrom's macroglobulinemia), cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, heavy chain disease, and solid tumors such as sarcomas and carcinomas, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilm's tumor, cervical cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma.

In one embodiment, the methods provided herein are used to treat a cancer selected from malignant pleural disease, mesothelioma, lung cancer (for example, non-small cell lung cancer), pancreatic cancer, ovarian cancer, breast cancer (for example, metastatic breast cancer, metastatic triple-negative breast cancer), colon cancer, pleural tumor, glioblastoma, esophageal cancer, gastric cancer, and synovial sarcoma. Provided herein are therapies that are particularly useful for treating solid tumors, for example, malignant pleural disease, mesothelioma, lung cancer, pancreatic cancer, ovarian cancer, breast cancer, colon cancer, pleural tumor, glioblastoma, esophageal cancer, gastric cancer, and synovial sarcoma. Solid tumors can be primary tumors or tumors in a metastatic state.

For treatment, the amount administered is an amount effective for producing the desired effect. An effective amount or therapeutically effective amount is an amount sufficient to provide a beneficial or desired clinical result upon treatment. An effective amount can be provided in a single administration or a series of administrations (one or more doses). An effective amount can be provided in a bolus or by continuous perfusion. In terms of treatment, an effective amount is an amount that is sufficient to palliate, ameliorate, stabilize, reverse or slow the progression of the disease, or otherwise reduce the pathological consequences of the disease. The effective amount can be determined by the physician for a particular subject. Several factors are typically taken into account when determining an appropriate dosage to achieve an effective amount. These factors include age, sex and weight of the subject, the condition being treated, the severity of the condition and the form and effective concentration of the cells of the invention being administered.

The cells provided herein are generally administered as a dose based on cells per kilogram (cells/kg) of body weight of the subject to which the cells are administered. Generally the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to 10⁶, depending on the mode and location of administration. In general, in the case of systemic administration, a higher dose is used than in regional administration, where the immune cells of the invention are administered in the region of a tumor. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 1×10⁸, 6×10⁴, to 1×10⁸, 7×10⁴ to 1×10⁸, 8×10⁴ to 1×10⁸, 9×10⁴ to 1×10⁸, 1×10⁵ to 1×10⁸, for example, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 3×10⁵ to 3×10⁶ cells/kg, and the like. Such dose ranges can be particularly useful for regional administration. In a particular embodiment, cells are provided in a dose of 1×10⁵ to 1×10⁸, for example 1×10⁵ to 1×10⁷, 1×10⁵ to 1×10⁶, 1×10⁶ to 1×10⁸, 1×10⁶ to 1×10⁷, 1×10⁷ to 1×10⁸, 1×10⁵ to 5×10⁶, in particular 1×10⁵ to 3×10⁶ or 3×10⁵ to 3×10⁶ cells/kg for regional administration, for example, intrapleural administration. Exemplary dose ranges also can include, but are not limited to, 5×10⁵ to 1×10⁸, for example, 6×10⁵ to 1×10⁸, 7×10⁵ to 1×10⁸, 8×10⁵ to 1×10⁸, 9×10⁵ to 1×10⁸, 1×10⁶ to 1×10⁸, 1×10⁶ to 9×10⁷, 1×10⁶ to 8×10⁷, 1×10⁶ to 7×10⁷, 1×10⁶ to 6×10⁷, 1×10⁶ to 5×10⁷, 1×10⁶ to 4×10⁷, 1×10⁶ to 3×10⁷ cells/kg, and the like. Such does can be particularly useful for systemic administration. In a particular embodiment, cells are provided in a dose of 1×10⁶ to 3×10⁷ cells/kg for systemic administration. Exemplary cell doses include, but are not limited to, a dose of 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹ and so forth in the range of about 10⁴ to about 10¹⁰ cells/kg. In addition, the dose can also be adjusted to account for whether a single dose is being administered or whether multiple doses are being administered. The precise determination of what would be considered an effective dose can be based on factors individual to each subject, including their size, age, sex, weight, and condition of the particular subject, as described above. Dosages can be readily determined by those skilled in the art based on the disclosure herein and knowledge in the art.

The cells provided herein can be administered by any methods known in the art, including, but not limited to, pleural administration, intravenous administration, subcutaneous administration, intranodal administration, intratumoral administration, intrathecal administration, intrapleural administration, intraperitoneal administration, intracranial administration, and direct administration to the thymus. In one embodiment, the cells provided herein can be delivered regionally to a tumor using well known methods, including but not limited to, hepatic or aortic pump; limb, lung or liver perfusion; in the portal vein; through a venous shunt; in a cavity or in a vein that is nearby a tumor, and the like. In another embodiment, the cells provided herein can be administered systemically. In a preferred embodiment, the cells are administered regionally at the site of a tumor. The cells can also be administered intratumorally, for example, by direct injection of the cells at the site of a tumor and/or into the tumor vasculature. For example, in the case of malignant pleural disease, mesothelioma or lung cancer, administration is preferably by intrapleural administration (see Adusumilli et al., Science Translational Medicine 6(261):261ra151 (2014)). One skilled in the art can select a suitable mode of administration based on the type of cancer and/or location of a tumor to be treated. The cells can be introduced by injection or catheter. In one embodiment, the cells are plurally administered to the subject in need, for example, using an intrapleural catheter. Optionally, expansion and/or differentiation agents can be administered to the subject prior to, during or after administration of cells to increase production of the cells provided herein in vivo.

Proliferation of the cells provided herein is generally done ex vivo, prior to administration to a subject, and can be desirable in vivo after administration to a subject (see Kaiser et al., Cancer Gene Therapy 22:72-78 (2015)). Cell proliferation should be accompanied by cell survival to permit cell expansion and persistence, such as with T cells.

The methods provided herein can further comprise adjuvant therapy in combination with, either prior to, during, or after treatment with the cells provided herein. Thus, the cell therapy methods provided herein can be used with other standard cancer care and/or therapies that are compatible with administration of the cells provided herein.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein, including the best mode known to the inventors for carrying out the invention. Upon reading the foregoing description, variations of the disclosed embodiments shall become apparent to individuals working in the art, and it is expected that those skilled artisans can employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### 7. EXAMPLES

### 7.1 Overview of Experiments

Allograft rejection is a main cause of the failure in allogeneic organ transplantation or allogeneic cell transplant. NK cell-mediated allogeneic responses, including cytolysis of transplanted cells, are important factors of this rejection (Li, Transplantation. 2010; 90(10): 1043-1047). Universal CAR-T therapies can potentially overcome drawbacks of autologous transplantation, but also face the challenge of allogeneic rejection. Studies described below illustrate the production of the genetically engineered cells disclosed herein that express fusion proteins comprising a presenting peptide covalently linked to human β2M. Some cells illustrated herein are T cells co-expressing a CAR that specifically recognize a tumor antigen. As provided in detail below, cells provided herein were shown to have better survival and enhanced proliferation in allogeneic environment. In particular, cells provided herein were shown to be resistant to NK cell-mediated cell lysis. Additionally, the CAR-T cells expressing the fusion proteins provided herein were also demonstrated to have greatly enhanced activities in inhibiting growth of tumor cells and promoting the death of tumor cells in an allogeneic environment.

The meanings of the abbreviations are as follows: “h” refers to hour, “min” refers to minute, “s” refers to second, “ms” refers to millisecond, “d” refers to day, “μL” refers to microliter, “mL” refers to milliliter, “L” refers to liter, “bp” refers to base pair, “mM” refers to millimole per liter, and “μM” refers to micromole per liter.

### 7.2 Materials and Methods

Generally, method for preparing an engineered cell comprises the steps: (i) expressing a fusion protein disclosed herein at the cell surface; the fusion protein comprising: (a) a presenting peptide; (b) a β2M peptide; and (c) a linker sequence covalently linking the aforementioned (a) and (b). The method can optionally further comprise: (ii) reducing the expression of an endogenous Class I MHC molecule at the cell surface. In some embodiments, the gene encoding the Class I MHC molecule is mutated or knocked out. The order of steps (i) and (ii) above can be interchanged.

Lentivirus preparation: a three-plasmid system can be used, including: a lentivirus target expression plasmid (Addgene ID: #12252) wherein the GFP expression cassette is replaced with various expression cassettes to express CAR and fusion proteins), and packaging helper plasmids psPAX2 (Addgene ID: #12260) and pMD2.G (Addgene ID: #12259). Virus packaging can be performed in HEK293T cells (Shanghai Institute of Cell Research, Chinese Academy of Sciences). The preparation process can be done as follows: the HEK293T cells in cryopreserved working cells are thawed, cultured in a DMEM medium (+10% FBS+1% P/S) (Cellgro 10-013-CMR) in a 10 cm culture dish, and the medium is changed 2 days of culturing. Once confluence is reached, the cells can be passaged (e.g. replate cells of 1 full plate to 5 plates), and the cells of the 4^{th} passage can be transfected with plasmids. Transfection can be done using PEI as a transfection reagent (e.g. the mass ratio of PEI to plasmid is 2:1). A mixture of plasmids and PEI is added to an Opti-MEM medium (Gibco, cat #31985-070), and then the mixed solution is added to the HEK293T cells of the 4^{th} passage. 6 hours after transfection, the medium is changed with a fresh medium containing 2% FBS, and then the cells are continuously cultured for 72 hours, when the supernatant of HEK293T cells is collected. The collected virus supernatant is concentrated by ultra-centrifugation (e.g. at a temperature of 2 to 8° C. at 82200 g for 2 hours), and the concentrated virus is sterilized by filtering through a 0.22 m filter membrane and resuspended for use.

Vector construction: a target gene (e.g. encoding a CAR alone or a CAR with a fusion protein disclosed herein) is cloned into a lentivirus vector. The lentivirus vector comprises a long terminal repeat 5′ LTR and truncated 3′ LTR, RRE, a rev response element (cPPT), a central termination sequence (CTS) and a WHV post-transcriptional regulatory element (WPRE). The fusion protein can be constitutively expressed by an EF-1a (elongation factor-1a) promoter and cloned to the lentivirus vector by BmHI and SalI digestion.

Primary T cell activation: Primary T cells can be derived from peripheral blood (PBMC) of healthy human volunteers. The medium used can be a complete medium, ImmunoCult™-XF T Cell Expansion Medium (Stem Cell Technology, cat #10981)+300 IU/ml IL2 (Cayan, cat #HEILP-0201c). T cells can be activated using Dynabeads (Thermo, cat #11141D), and the ratio of Dynabeads to cells is 3:1. 24 hours after being activated, the T cells are visibly agglomerated and enlarged.

Lentivirus transduction of T cells: after the primary T cells are activated, from 24 hours to 72 hours, the cells are agglomerated and enlarged, and are in a mitotic phase for lentivirus transduction. The ratio of virus transducing units to number of cells can be 10:1, i.e., MOI=10. The protein expression detection can be performed 48 hours after transduction.

Gene editing: 4 days after the T cells are activated, or 2 days after the T cells are transduced with the virus, the cells are collected and washed 3 times with an electroporation buffer (Thermo, Resuspension Buffer T in the kit Cat #MPK1096), Opti-MEM (Gibco, cat #31985-070), or the like. The cells are resuspended in the electroporation buffer, and the cell density is adjusted to 1×10⁸/ml. The desired sgRNAs (300 to 400 ng in total) and 1 μg of Cas9 protein are uniformly mixed in vitro at the same time, and incubated for 10 min at room temperature, and then the mixture is added to the resuspended cells. The total volume of the cells for electroporation can be 10 μL, and a Neon electroporation system (Thermo, Cat #MPK1096) can be used for electroporation. The pulse voltage, pulse duration, and number of pulses of the electric shock can be as follows: 1200 to 1600 v, 10 ms, and 3 times. An sgRNA candidate sequence can be designed according to the predictive analysis of the related website; the Cas9 protein can be derived from an Alt-R s.p. Cas9 Nuclease 3NLS protein (IDT, Cat #1074181) of IDT DNA Technology Company.

UCAR-T cell expressing the fusion protein: T cell activation, virus transduction, and gene editing methods are described as above. An exemplary process is as follows: on day 0, the primary PBMC activation is performed; on day 2, the virus transduction (for expressing the fusion protein) is performed, on day 4, the gene editing (TRAC and β2M are knocked out) is performed, and on day 6 to 7, the expression of each protein is detected by flow cytometry.

Materials: Exemplary antibodies and cellular dyes that can be used: anti-CD3-APC (BD, 555335), anti-CD3-FITC (BD, 555916), anti- β2M-PE (BD, 551337), anti- β2M-FITC (BD, 551338), anti-HLA-ABC-APC (R&D, FAB7098A), anti-HLA-E-PE (Biolegend, 342604), anti-HLA-E-APC (Biolegend, 342606), anti-Mouse FMC63 (Shanghai Xingwan, R19PB-100), and Dye eFluor™ 670 (eBioscience, 65-0840-90), anti HLA-ABC PE-Cy7 (Invitrogen, 25-9983-42), anti αβ-TCR PE (Biolegend, 306708).

### 7.3 Production of Universal CAR-T Cells Expressing a Fusion Protein (“UCAR-T-CM”)

### Construction of CAR19-P2A-CM, CAR19-P2A-CM-c, and CAR19 Lentivirus Target Expression Plasmids:

A lentivirus target expression plasmid with CAR19 (i.e. a CAR comprising an antigen binding domain that specifically binds tumor antigen CD19) alone or CAR 19 with a fusion protein (or a “chimeric molecule,” a “CM”) was constructed. The fusion protein (CM) used here has the following components from N terminus to C terminus: a 20-a.a. signal peptide (SEQ ID NO:3), a presenting peptide, a linker sequence GGGGSGGGGSGGGGS (SEQ ID NO:1), a β2M mature form peptide (SEQ ID NO:81). The CAR19 and CM were linked by a sequence encoding a P2A peptide (ATNFSLLKQAGDVEENPGP; SEQ ID NO:105), which is a self-cleaving polypeptide commonly used to simultaneously express two or more independent polypeptides from the same vector. After being co-expressed from the same plasmid, self-cleavage of the P2A peptide allowed the two polypeptides linked thereto to be separated and function independently. For CAR19-P2A-CM, the fusion protein (CM) as seen in SEQ ID NO:5 comprised an HLA-E restricted presenting peptide (VMAPRTVLL; SEQ ID NO:38). The nucleic acid sequence encoding CAR19-P2A-CM is SEQ ID NO:12. CAR19-P2A-CM-c was otherwise the same as CAR19-P2A-CM, except that the fusion protein (CM-c) comprised an HLA-C restricted presenting peptide IIDKSGSTV (SEQ ID NO:117). The nucleic acid sequence encoding CAR19-P2A-CM-c is SEQ ID NO:19.

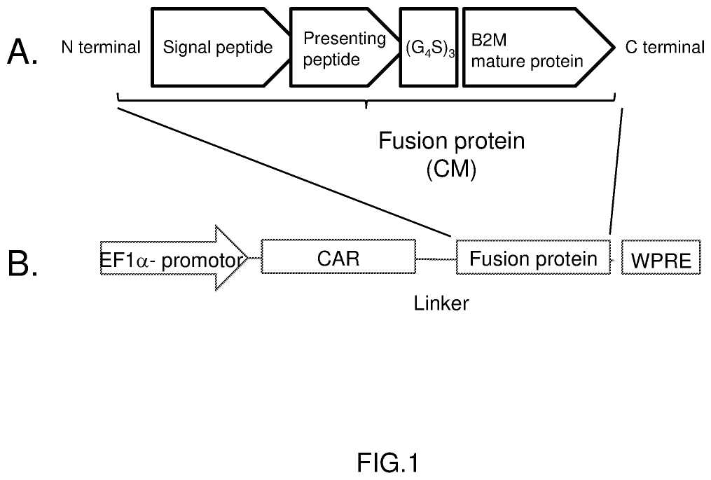

A virus expression plasmid (Addgene ID: #12252) was digested with BamHI/SalI ahd served as a backbone. A CAR19 sequence (SEQ ID NO:20), a CAR19-P2A-CM sequence (SEQ ID NO:12), or a CAR19-P2A-CM-c sequence (SEQ ID NO:19) was cloned into the backbone, forming the CAR19-P2A-CM, CAR19-P2A-CM-c, and CAR19 expression plasmids, respectively. As shown in FIG. 1, the plasmid comprising the nucleotide sequence encoding CAR19-P2A-CM (SEQ ID NO:12) was operably inserted into the digested virus expression backbone.

Lentivirus preparation: A three-plasmid system was used, which consisted of: a lentivirus target expression plasmid constructed by the methods described above, and packaging helper plasmids psPAX2 (Addgene ID: #12260) and pMD2.G (Addgene ID: #12259). Virus packaging was performed in HEK293T cells (Shanghai Institute of Cell Research, Chinese Academy of Sciences). The preparation process was as follows: the HEK293T cells in cryopreserved working cells were thawed, cultured in a DMEM medium (+10% FBS+1% P/S) (Cellgro 10-013-CMR) in a 10 cm culture dish, and the medium was changed after 2 days of culturing. The cells were passaged when confluence was reached (1 full plate of cells were passaged to 5 new plates), and the cells of the 4^{th} passage could be transfected with plasmids. Polyethylenimine (PEI) was used as the transfection reagent with the mass ratio of PEI to plasmid being 2:1. A mixture of the three plasmids and PEI was then added to an Opti-MEM medium (Gibco, cat #31985-070), and the mixed solution was added to the HEK293T cells of the 4^{th} passage. 6 hours after transfecting, the medium was changed with a fresh medium containing 2% FBS, and then the cells were continuously cultured for 72 hours before supernatant of the cell cultures was collected. The virus in the collected supernatant was concentrated by ultra-centrifugation (at 4 to 8° C. at 82200 g for 2 hours), and the concentrated virus was sterilized by filtering through a 0.22 m filter membrane and resuspended for use.

Primary T cell activation: Primary T cells were derived from peripheral blood (PBMC) of healthy human volunteers. A complete medium was used, namely, ImmunoCult™-XF T Cell Expansion Medium (Stem Cell Technology, cat #10981)+300 IU/ml IL2 (Cayan, cat #HEILP-0201c). T cells were activated using Dynabeads (Thermo, cat #11141D), and the ratio of Dynabeads to cells was 3:1. 24 hours after being activated, the T cells were visibly agglomerated and enlarged.

Lentivirus gene transduction: 3×10⁵ primary T cells that had been activated for 48 hours, or Jurkat cells (Shanghai Institute of Cell Research, Chinese Academy of Sciences) without activation, were seeded in a 24-well culture plate, the prepared lentivirus was added in an amount 10 times the number of cells, i.e., MOI=10 (“multiplicity of infection,” the ratio of virus transducing units to cell number), and the medium was supplemented to bring the volume to 500 μL (the medium was identical to the medium used for the activation of the aforementioned primary T cells). After 24 h, the medium was supplemented to bring the volume to 1 mL to facilitate cell growth.

Candidate sgRNA: sgRNAs editing B2M gene and sgRNA editing TRAC gene were designed and screened. sgRNAs (SEQ ID NO:8; SEQ ID NO:9) were used to edit B2M gene and an sgRNA (SEQ ID NO:10) was used to edit TRAC gene.

Cas9 protein: Alt-R s.p. Cas9 Nuclease 3NLS protein from the IDT DNA technology company was used.

Electroporation: 48 h after the primary T cells or Jurkat cells were transduced with the lentivirus, the cells were collected, washed 3 times with an electroporation buffer (Thermo, Resuspension Buffer T in the kit Cat #MPK1096) or Opti-MEM (Gibco, cat #31985-070), and resuspended in the electroporation buffer. The cell density was adjusted to 1×10⁸/ml. The β2M sgRNAs and TRAC sgRNA (150 ng respectively) and 1 μg of Cas9 protein were uniformly mixed in vitro, incubated for 10 min at room temperature, and then the mixture was added to the resuspended cells for electroporation. The total volume for electroporation was 10 μL, and a Neon electroporation system was used. The pulse voltage, pulse duration, and number of pulses of electric shock were as follows: 1200 V, and 10 ms, 3 times.

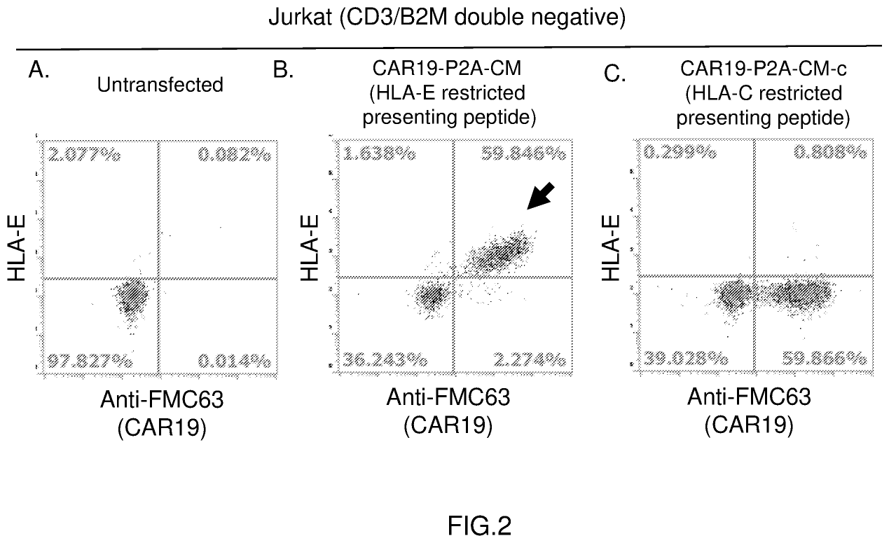

Measurement of virus transduction efficiency and gene editing efficiency: 3 days after the electroporation was completed, the efficiency of genetic engineering was measured. A small amount of cells were taken out, and washed once with 1 ml of PBS (Gibco, cat #C10010500BT). The cells were resuspended in 100 μL of PBS, and 3 μL of a primary antibody of an anti-FMC63 antibody (detecting CAR19) was added, and the mixture was incubated for 30 min at 4° C. Then 1 ml PBS was added to the mixture, which was uniformly mixed, and centrifuged at 350 g for 3 min. The cells were collected and the supernatant was discarded. Sequentially, 0.5 μL secondary antibody (the primary antibody and secondary antibody were from an anti-Mouse FMC63 kit, Shanghai Xingwan, R19PB-100) and 3 μL anti-HLA-E-APC antibody were added to the cells, which were uniformly mixed, incubated at 4° C. for 30 min, added with 1 ml of PBS to be uniformly mixed, and centrifuged at 350 g for 3 min. The cells were collected and the supernatant was discarded, 200 μL PBS was added to resuspend the cells, and the cells were analyzed by a flow cytometer. As shown in FIG. 2, all Jurkat T cells had TRAC and β2M knocked out using CRISPR/Cas9 gene editing as described above. When these cells were transduced with the CAR19-P2A-CM virus (FIG. 2, panel B), the fusion protein having the HLA-E specific presenting polypeptide (CM) was efficiently expressed in cells expressing CAR on the surface (cells in the upper-right quadrant), since the fusion protein and CAR were operatively linked together. The CAR-positive cells in Panel B were also stained positive for HLA-E heavy chain, showing that the CM was bound to the HLA-E heavy chain to form a complex on the cell membrane, since CM lacks the HLA-E heavy chain. Meanwhile, in cells that were transduced with the CAR19-P2A-CM-c virus and had TRAC and β2M knocked out, only the CAR expression, but not the HLA-E heavy chain expression was detected on cell surface, indicating that the fusion protein expressing the HLA-C restricted presenting polypeptide (CM-c) failed to form a complex with endogenous HLA-E heavy chain (FIG. 2, panel C).

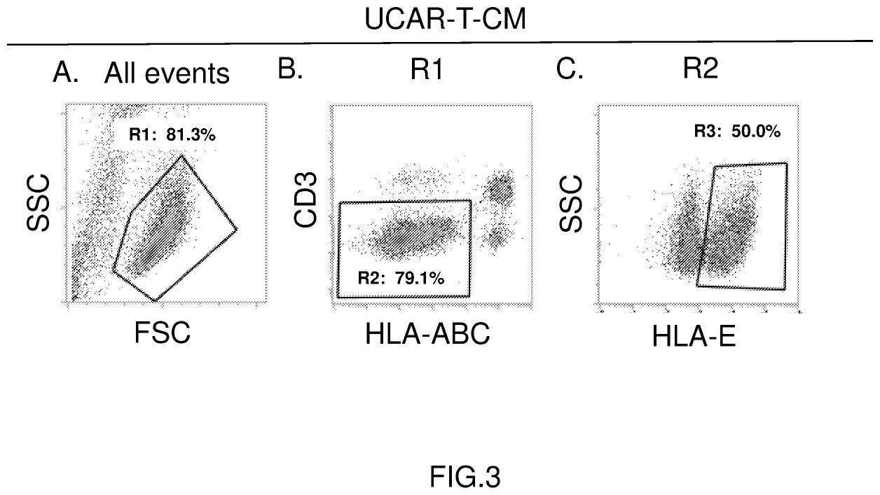

For measuring gene editing efficiency in primary T cells, a small amount of cells was taken out, washed once with 1 ml PBS, and resuspended in 100 μL PBS. 3 μL of an anti-HLA-E-PE antibody, 3 μL of an anti-HLA-ABC antibody, and 3 L of an anti-CD3-FITC antibody were added. As shown in FIG. 3, panel B, after lentiviral transduction and gene editing as described above, about 80% of primary T cells transduced with the CAR19-P2A-CM virus and having the double knockout of TRAC and β2M (i.e. UCAR-T-CM cells) were double negative for CD3 and HLA-ABC. These double-negative cells also had high expression of the fusion protein containing the HLA-E restricted presenting peptide (CM), which was demonstrated by the detection of CM-bound HLA-E at the cell membrane (FIG. 3, panel C). The 50% HLA-E positive cells detected by the anti-HLA-E antibody in CD3 and HLA-ABC double-negative cells indicated the expression of the fusion protein, since these cells had endogenous β2M knocked out, HLA-E heavy chain must bind to recombinant CM to be presented at the cell surface and be detected by an anti-HLA-E antibody. Thus, these results showed that the gene editing of β2M efficiently removed the presentation of the endogenous HLA-ABC complexes on the cell membrane, the gene editing of TRAC efficiently removed the presentation of TCR-CD3 complex on the cell membrane, the fusion protein was efficiently expressed and formed stable complex with HLA-E heavy chain on the cell surface.

### 7.4 UCAR-T Cells Expressing Fusion Proteins (CM) were Resistant Against Allogeneic NK Cells

Measurement of NK92 cell (a human NK cell line)-mediated cytolysis by flow cytometry. ImmunoCult™ (STEMCELL) was used as the culture medium as described above. NK92 cells (Shanghai Institute of Cell Research, Chinese Academy of Sciences) were used as effector cells. Three (3) types of target cells were used, all generated from primary T cells derived from peripheral blood of healthy human volunteers as described above, including: 1. CAR-T cells, which were the primary T cells transduced with the CAR19 virus; 2. UCAR-T cells, which were the primary T cells transduced with the CAR19 virus and having the TRAC and B2M genes knocked out; and 3. UCAR-T-CM cells, which were the primary T cells transduced with the CAR19-P2A-CM virus and having the TRAC and B2M genes knocked out. The final concentration of all target cells was 5×10⁴/mL.

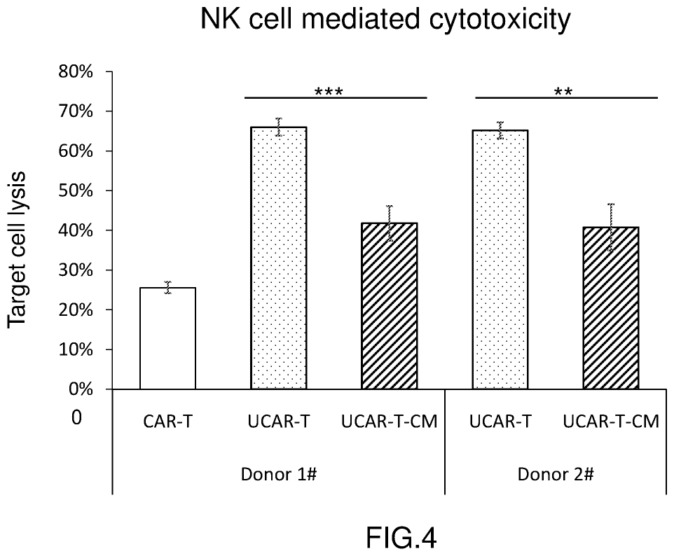

In each group, the NK92 cells as effector cells and the target cells were co-cultured at a ratio of 1:1 (cell number ratio). Each group of the target cells were also cultured alone as control. Cells were seeded in 96-well culture plates, supplemented with 100 μL of culture medium, and co-cultured in a 37° C. incubator for 24 hours. Prior to co-culture, the NK92 cells were stained with Dye eFluor™ 670 to be distinguished from the target cells. 48 h after co-culture, the changes in target cell numbers in each of the groups of CAR-T cells, UCAR-T cells, or UCAR-T-CM cells were measured by detecting CAR-positive cells by flow cytometry. The anti-Mouse FMC63 kit was used for staining CAR-positive cells as described above. The killing rate of the target cells by the NK92 cells was calculated as: (number of CAR-positive cells in co-culture group)/(number of CAR-positive cells in target cell group only). As shown in FIG. 4, UCAR-T-CM cells from both volunteers (donor 1 # and donor 2 #) showed significant resistance to NK cell-mediated cytolysis (**p<0.05, *** p<0.01).

### 7.5 UCAR-T Cells Expressing Fusion Proteins (CM) Rapidly Expanded in the Presence of Allogeneic PBMC

### Co-Culture of UCAR-T Cells and Allogeneic PBMC Cells:

This study further simulated the in vivo environment. Because human bodies have a complex system comprising many different types of immune cells in addition to NK cells, the primary allogeneic PBMCs were used to closely simulate the environment of an allogeneic immune system in human body. In order to measure the ability of the genetically engineered cells to survive and grow in the allogeneic PBMC environment, this study co-cultured three types of cells together, the UCAR-T cells or UCAR-T-CM cells as effector cells, the Raji tumor cells (Shanghai Institute of Cell Research, Chinese Academy of Sciences) as the target cells, and the allogeneic PBMCs from peripheral blood of healthy human volunteers as a mimic of an allogeneic immune environment. The primary PBMCs and T cells used for preparing UCAR-T or UCAR-T-CM were obtained from different donors to elicit allogeneic responses. Raji tumor cells were CD19 positive, so T cells containing CAR19 were activated by the CD19 antigen on Raji cells and started to proliferate.

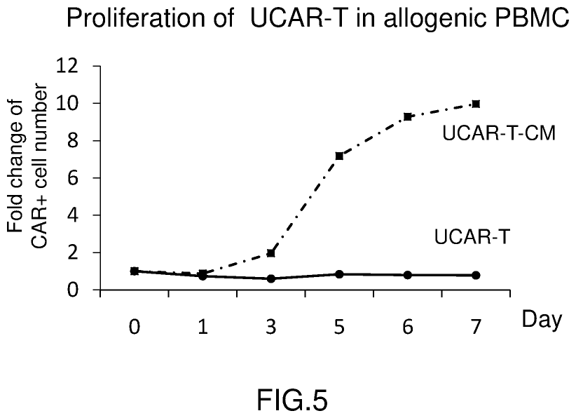

The cells were co-cultured according to the following ratio: CAR-positive cells in UCAR-T cells (or UCAR-T-CM cells): Raji tumor cells: allogeneic PBMCs is 1:5:20 (cell number ratio). 5×10⁴/ml CAR-positive cells in the UCAR-T cells (or UCAR-T-CM cells) were seeded in a 24-well culture plate, and appropriate amounts of the Raji tumor cells and allogeneic PBMCs were calculated according to the above ratio (i.e. 2.5×10⁵/ml Raji tumor cells and 1×10⁶/ml allogeneic PBMCs) and seeded. The cells were cultured at 37° C., and the numbers of CAR-positive cells in different groups were recorded at different time points. The number of CAR-positive cells at different time points was divided by the number of starting CAR-positive cells on day 0 to obtain the cell expansion fold changes (FIG. 5). The same staining method of CAR-positive cells (using anti-Mouse FMC63 kit) was used.

Allogeneic PBMCs and Raji cells were stained with Dye eFluor™ 670 prior to co-culture to be distinguished from the UCAR-T cells or UCAR-T-CM cells. To stain the cells, the cell density was adjusted to 1×10⁷/ml, Dye eFluor™ e670 was added to a final concentration of M, and the mixture was incubate at room temperature for 5 min in the dark, and wash with the medium three times before using.

FIG. 5 shows that in the presence of the allogeneic PBMC cells, UCAR-T cells were not able to expand well with the stimulation of CD19-positive Raji cells, whereas UCAR-T-CM cells were able to expand rapidly. These results indicated that allogeneic immune cells in PBMC inhibited the growth of UCAR-T cells, but not the UCAR-T-CM cells, upon stimulation by cancer cells, again demonstrating that CAR-T cells genetically engineered to express the fusion protein (CM) were able to overcome inhibition and/or rejection by allogeneic immune cells, and to survive and grow in an allogeneic environment.

### 7.6 UCAR-T Cells Expressing Fusion Proteins (CM) Efficiently Caused Death of Tumor Cells in the Presence of Allogeneic PBMCs

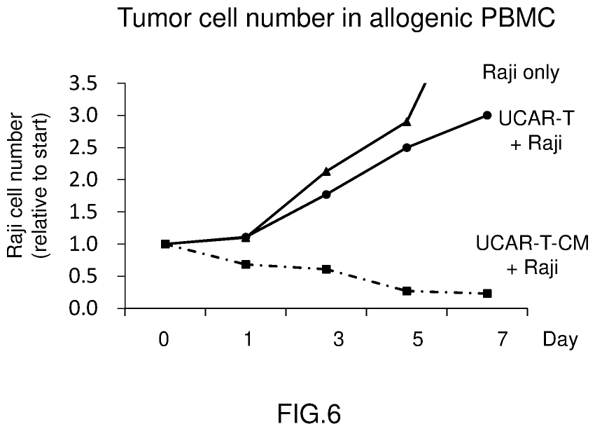

The experimental procedure was the same as described above in section 7.5, but different cells were measured. Instead of the numbers of CAR-positive cells, the numbers of Raji cells in different groups were measured at different time points in this experiment. The Raji cells were stained with the dye e670 prior to co-culture, which could be distinguished from UCAR-T or UCAR-T-CM via APC fluorescence channels in flow cytometry. The Raji cells and the PBMC cells could be clearly distinguished in flow cytometry analysis, because that the Raji cells are tumor cells and have significantly different morphology and size from PBMC cells, and also because that the continuous expansion of tumor cells during co-culture led to the continuous decrease of fluorescence of the dye e670. Fold changes of Raji cells at different time points were calculated by dividing the numbers of Raji cells at different time points by the numbers of starting Raji cells on day 0. As shown in FIG. 6, the number of tumor cells in a Raji-alone culture group was increased with time, and the UCAR-T cells had weak inhibition on tumor cells in the presence of allogeneic PBMCs—the tumor cells still increased. By contrast, growth of tumor cells co-cultured with UCAR-T-CM cells was significantly inhibited and these tumor cells were gradually cleared. The results corroborated with the results described above in section 7.5, and demonstrated that the UCAR-T-CM cells that were able to expand rapidly when stimulated by tumor cells were also active in killing tumor cells.

### 7.7 Detection of a Complex Formed by a Fusion Protein (CM) and an HLA-E Heavy Chain on Cell Membrane of P2M-Null Cells

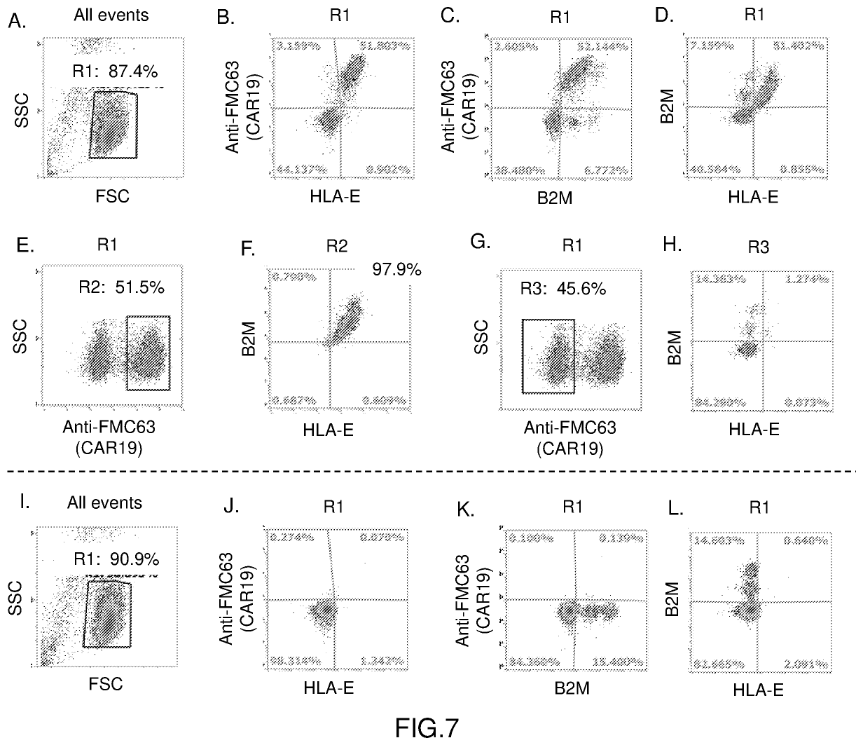

The experimental procedure as described above was followed, and Jurkat T cells were used in this study. Formation of a complex having a fusion protein (CM) and an endogenous HLA-E heavy chain molecule on the cell membrane was detected by flow cytometry (FIG. 7).

The Jurkat cells in each group were stained with the anti-Mouse FMC63 kit for detecting CAR, and anti-β2M-FITC and anti-HLA-E-APC, for detecting β2M and HLA-E, respectively. A small amount of cells were taken, washed once with 1 ml of PBS (Gibco, cat #C10010500BT), and resuspended in 100 μL of PBS. 3 μL of a primary anti-FMC63 antibody (for detecting CAR19) was added to the cell mixture, which was incubated for 30 min 4° C. Then 1 ml of PBS was added and the cell mixture was uniformly mixed and centrifuged at 350 g for 3 min. The cells were collected and the supernatant was discarded. Sequentially, 0.5 μL of a secondary antibody (the primary antibody and secondary antibody were from the anti-Mouse FMC63 kit, a product of Shanghai Xingwan, R19PB-100), 3 μL of an anti-β2M-FITC antibody, and 3 μL of an anti-HLA-E-APC antibody, were added, and the mixture was uniformly mixed, incubated at 4° C. for 30 min. 1 ml of PBS was added and uniformly mixed, and the cell mixture was centrifuged at 350 g for 3 min. The cells were collected and the supernatant was discarded. 200 μL of PBS was added to resuspend the cells, which were loaded in the flow cytometer for detection.

The flow cytometry data of FIGS. 7A-H were from Jurkat cells that were transduced with the CAR19-P2A-CM virus and had endogenous TRAC gene and B2M gene knocked out. The flow cytometry data of FIGS. 7I-L were from the control group, in which the Jurkat cells had endogenous TRAC gene and B2M gene knocked out but were not transduced with the CAR19-P2A-CM virus.

FIG. 7, panel B shows the only the CAR-expressing cells were HLA-E positive. Since the nucleic acid encoding CAR and the nucleic acid encoding the fusion protein (CM) were cloned to be operably linked on the same viral vector (via a nucleotide sequence encoding a P2A peptide), the cell expressing CAR also expressed CM. Therefore, only the CM-expressing cells were HLA-E positive. FIG. 7, panel C shows the co-expression of the CAR and the fusion protein (CM), since CM comprised β2M, CAR and β2M double positive cells were observed. FIG. 7, panel D shows the co-localization of β2M and HLA-E. Since most cells lacked endogenous expression of β2M, anti-β2M antibody actually detected CM which contained recombinant β2M. The fact that HLA-E could only be detected on the cell surface of CM-expressing cells demonstrated that recombinantly expressed CM and the endogenous HLA-E heavy chain formed a complex on cell surface.

FIG. 7, panel F shows that the CAR-positive cell population R2 almost exclusively included the β2M and HLA-E double positive cells, while FIG. 7, panel H shows that β2M and HLA-E double positive cells were not detected in CAR-negative cells. These results further demonstrated that β2M signal was generated from the CM molecule co-expressed with the CAR, and that the CM (containing recombinant β2M and an HLA-E restricted peptide) formed a complex with HLA-E heavy chain in the β2M and HLA-E double positive cells. β2M expression is required for membrane presentation of HLA-E heavy chain. When CM was not expressed (in the CAR-negative cells), HLA-E could not be detected on the cell surface of β2M-knockout Jurkat cells. In those β2M-knockout Jurkat cells, when CM was expressed (in the CAR-positing cells), CM and the HLA-E heavy chain formed a complex that restored the presentation of the HLA-E heavy chain on the cell membrane, so that the HLA-E heavy chain molecule could be detected at the cell surface.

FIG. 7, panels I-L shows that in the cells of the control group, no CAR or HLA-E was detected at the cell surface. The small amount of β2M-positive cells represented the cells in which β2M was not completely knocked out by gene editing.

### 7.8 Expression of Different Presenting Peptides were Associated with Different Expression of HLA-E Molecule on Cell Membrane

Presenting peptides affect the binding between fusion protein (CM) and HLA-E heavy chain, which in turn affects the expression of the HLA-E complex on cell membrane. 18 different presenting peptides were (P1-P18; SEQ ID NOs: 21-38) selected and cloned into lentivirus expression vector as GFP-P2A-CMx (x for different peptide segments number P1-P18). The fusion protein CMx used here has the following components from N terminus to C terminus: a 20-a.a. signal peptide (SEQ ID NO:3), a presenting peptide x selected from P1-P18 (SEQ ID NOs: 21-38), a linker sequence GGGGSGGGGSGGGGS (SEQ ID NO:1), and a β2M mature form peptide (SEQ ID NO:81). The methods for construction of lentivirus target expression plasmids were the same as described above, using BamHI/SalI to clone GFP-P2A-CMx into the lentivirus vector. The viruses were transduced into Jurkat cells that had β2M knocked out. Methods of lentivirus preparation and virus preparation were the same as described above.

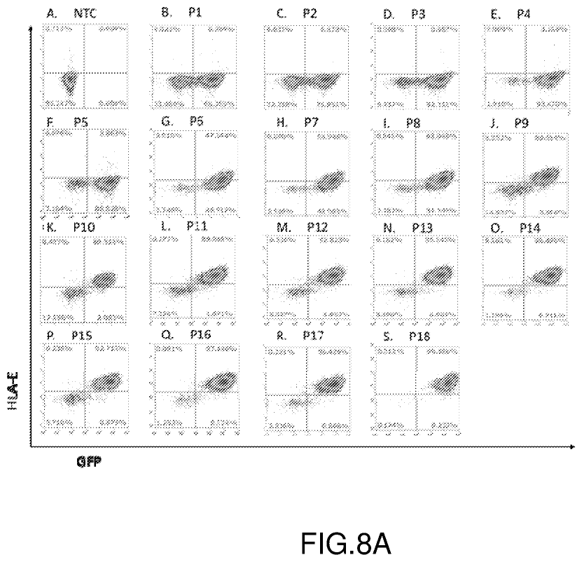

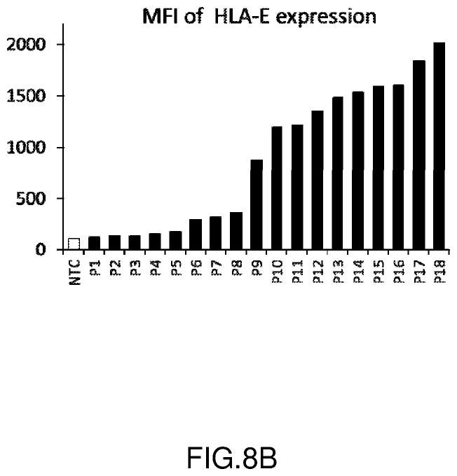

Expression of HLA-E complex on cell membrane was detected with anti-HLA-E-APC antibody (Biolegend, 342606) and by measuring GFP, which indicated the lentiviral transduction efficiency. As shown in FIG. 8A (quantified in FIG. 8B), expression of different presenting peptides were associated with different levels of expression of the HLA-E complex on cell membrane (NTC, Negative Control; untransfected group).

### 7.9 Various Fusion Proteins Expressed Efficiently in UCAR-T Cells, and UCAR-T Cell Expansion in the Presence of Allogeneic PBMCs Increased with the Expression of Fusion Proteins

Construction of CJP-P2A-CM, CJP-P2A-CM12, CJP-P2A-CM13, CJP-P2A-CM14, and CJP Lentivirus Target Expression Plasmids, and Preparation of UCAR-T Cells with or without Fusion Protein

The procedures of constructing lentivirus target expression plasmids, preparing UCAR-T cells expressing different fusion proteins, and measuring cell surface expression using flow cytometry were the same as described in sections 7.2 and 7.3 above. CJP is an anti-CD19 CAR (SEQ ID NO:75). The fusion proteins were the same as those described in section 7.8 above. Amino acid sequences of these constructs are summarized below. Nucleotide sequences can be found in the Sequence Listing.

| | Fusion Protein | Presenting Peptide |
|---|---|---|
| CJP-P2A-CM | CM | B7 |
| (SEQ ID NO: 124) | (SEQ ID NO: 5) | (SEQ ID NO: 38) |
| CJP-P2A-CM12 | CM12 | G |
| (SEQ ID NO: 126) | (SEQ ID NO: 15) | (SEQ ID NO: 32) |
| CJP-P2A-CM13 | CM13 | A2 |
| (SEQ ID NO: 128) | (SEQ ID NO: 14) | (SEQ ID NO: 33) |
| CJP-P2A-CM14 | CM14 | Cw3 |
| (SEQ ID NO: 130) | (SEQ ID NO: 13) | (SEQ ID NO: 34) |

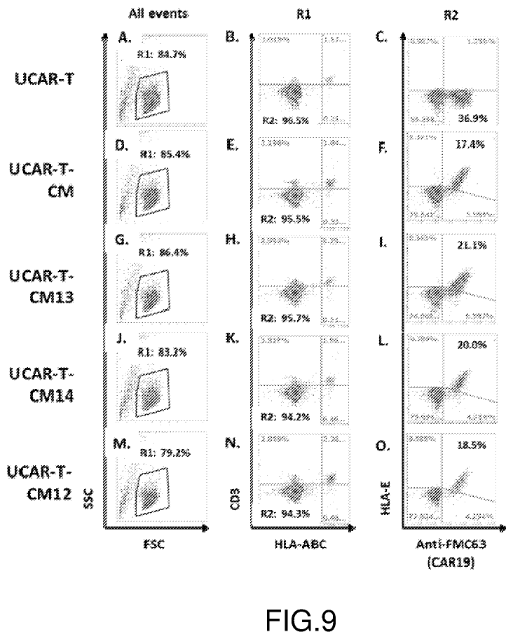

Highly efficient knock-out of TRAC and B2M genes were indicated by flow cytometry analysis of CD3 and HLA-ABC expression on the cell surface, respectively (FIG. 9, panels B, E, H, K, N). All groups had >85% of CD3 and HLA-ABC double-negative cells. HLA-E and CAR were co-detected on the cell surface of UCAR-T-CM, UCAR-T-CM12, UCAR-T-CM13, and UCAR-T-CM14, each containing a different presenting peptide as shown in the table above (FIG. 9, panels C, F, I, L, O, analysis done on gated CD3 and HLA-ABC double-negative cells). The results indicated that compared to the control UCAR-T group without any fusion protein, all of these fusion proteins having different HLA-E restricted peptides were able to restore the surface expression of HLA-E complex, but not HLA-ABC complex in TRAC and β2M double-knock-out T cells.

Expansion of UCAR-T Cells with Different Fusion Protein (CM, CM12, CM13, CM14) in the Presence of Allogeneic PBMCs

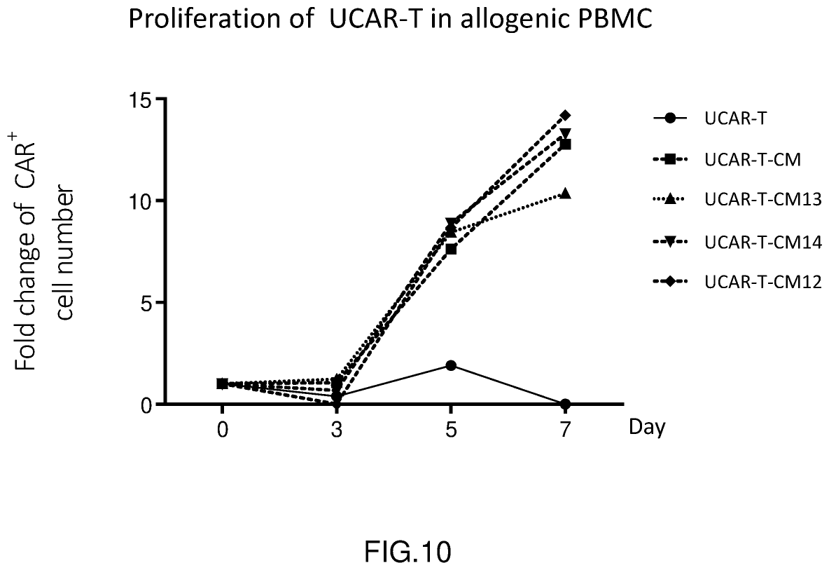

Experiment procedures were the same as described in Section 7.5 above. FIGS. 10A-10C show that in the presence of allogeneic PBMC cells, UCAR-T cells with no fusion protein expression were not able to expand well over time with the stimulation of CD19-positive Raji cells. In contrast, UCAR-T-CM, UCAR-T-CM12, UCAR-T-CM13, UCAR-T-CM14 cells were able to expand rapidly even in the presence of the allogeneic PBMC cells. Different population of PBMC cells were used in parallel experiments, including PBMC (FIG. 10). These results indicated that immune cells in allogeneic PBMCs inhibited the growth of UCAR-T cells, but not that of the UCAR-T-CM/CM12/CM13/CM14 cells, again demonstrating that T cells genetically engineered to express various forms of the fusion proteins disclosed herein were able to overcome inhibition and/or rejection by allogeneic immune cells, and to survive and grow in an allogeneic environment.

7.10 Exogenous NY-ESO-1 Targeted TCR was Co-Expressed with the Fusion Protein in Genetically Engineered Cells

A lentivirus target expression plasmid containing an exogenous NY-ESO-1 targeted TCR (1G4, SEQ ID NO:132) and the fusion protein CM (SEQ ID NO:5) was constructed, and cells were prepared using this lentiviral vector and CRISPR/Cas9 gene editing. The procedure of constructing the lentivirus plasmid with both 1G4 and CM linked by a P2A peptide was the same as described in sections 7.2 and 7.3 above. The lentivirus plasmid backbone (Addgene ID: #12252) was digested with BamHI/SalI. Construct 1G4-P2A-CM had the nucleotide sequence of SEQ ID NO:135, and the amino acid sequence of SEQ ID NO:134.

Preparation of cells with lentiviral transduction and CRISPR/Cas9 gene editing, and flow cytometry analysis of the cells after immunofluorescence staining were done as described in sections 7.2 and 7.3 above. For flow cytometry analysis, a small number of cells were taken out, and washed once with 1 ml of PBS (Gibco, cat #C10010500BT). The cells were resuspended in 100 μL of PBS, and 3 μL each of the following antibodies were added: anti-CD3-FITC, anti αβ-TCR PE, anti HLA-ABC PE-Cy7, anti-HLA-E-APC. After 30 min incubation at 4° C., 1 ml PBS was added to the mixture, which was uniformly mixed, and centrifuged at 350 g for 3 min. The cell pellets were collected, and the supernatant was discarded. 200 μL PBS was then added to resuspend the cells, and the cells were analyzed by a flow cytometer.

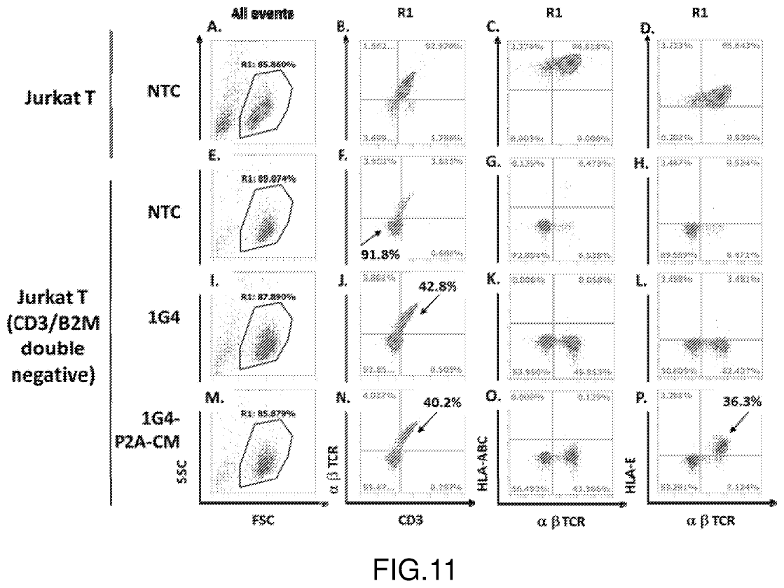

Surface expression of different molecules on resulting cells is shown in FIG. 11: panels A-D show results from wild-type Jurkat T cells; panels E-P show results from Jurkat T cells with TRAC and β2M double knock-outs (dKO), hence CD3 and β2M double negative. Among panels E-P of FIG. 11: panels E-H show results from Jurkat T cells with TRAC and B2M dKO with no lentiviral transduction; panels I-L show results from Jurkat T with TRAC and B2M dKO cells transduced with lentiviral vector carrying 1G4 TCR (SEQ ID NO:132); panels M-P show results from Jurkat T cells with TRAC and B2M dKO transduced with lentiviral vector 1G4-P2A-CM (1G4 TCR and the fusion protein CM linked by a P2A peptide; SEQ ID NO:134).

As shown in FIG. 11, the wild-type Jurkat T cells showed positive staining for CD3, αβ-TCR, HLA-ABC, and HLA-E (panels A-D). TRAC and β2M knocked-out Jurkat T cells had 91.8% CD3 and αβ-TCR double-negative cells (panel F, lower-left), and due to the lack of β2M, HLA-ABC and HLA-E could not be detected on the cell surface (panels G, H). Double-knocked-out Jurkat T cells transduced with 1G4 TCR showed 42.8% CD3 and αβ-TCR double-positive cells (panel J, upper-right), indicating that the expression of this exogenous TCR, namely 1G4, restored the surface presentation of the CD3/TCR complex; but HLA-ABC and HLA-E remained negative in this group (panels K, L). Double-knocked-out Jurkat T cells transduced with 1G4-P2A-CM showed 40.2% CD3 and αβ-TCR double-positive cells (panel N, upper-right), which was comparable to the group expressing 1G4 alone (panel J, upper-right); the expression of the fusion protein CM restored the surface presentation of HLA-E complex (panel P, upper-right, 36.3% HLA-E and αβ-TCR double-positive), but not HLA-ABC complex (panel O).

### 7.11 Expansion of Genetically Engineered NY-ESO-1 Targeted TCR-T Cells with or without Fusion Protein in the Presence of Allogeneic PBMCs

Preparation of Genetically Engineered NY-ESO-1 Targeted TCR-T Cells with or without the Fusion Protein CM

The procedures of constructing lentivirus target expression plasmids, activating primary T cells, gene editing, lentivirus-mediated gene transduction and measuring cell surface expression using flow cytometry were the same as described in sections 7.2, 7.3, and 7.10 above. Lentiviruses used were the same as described in section 7.10 above. Briefly, Primary T cells were derived from peripheral blood (PBMC) of healthy human volunteers. T cells were activated using Dynabeads (Thermo, cat #11141D), and the ratio of Dynabeads to cells was 3:1. 72 hours after being activated, the T cells were collected, washed 3 times with an electroporation buffer (Thermo, Resuspension Buffer T in the kit Cat #MPK1096) or Opti-MEM (Gibco, cat #31985-070), and resuspended in the electroporation buffer (Dynabeads were removed). The cell density was adjusted to 1×10⁸/ml. To prepare the β2M/TCR double knocked-out T cells, the β2M sgRNAs and TRAC sgRNA (150 ng respectively) and 1 μg of Cas9 protein were uniformly mixed in vitro, incubated for 10 min at room temperature, and then the mixture was added to the resuspended cells for electroporation. The total volume for electroporation was 10 L, and a Neon electroporation system was used. The pulse voltage, pulse duration, and number of pulses of electric shock were as follows: 1200 V, and 10 ms, 3 times. 24 hours after electroporation, 3×10⁵ primary T cells were seeded in a 24-well culture plate, the prepared lentivirus was added in an amount 10 times the number of cells, i.e., MOI=10 (“multiplicity of infection,” the ratio of virus transducing units to cell number), and the medium was supplemented to bring the volume to 500 μL (the medium was identical to the medium used for the activation of the aforementioned primary T cells). For 1G4 cells, lentivirus carrying 1G4 was used in the transduction; for 1G4-P2A-CM cells, lentivirus carrying 1G4-P2A-CM was used in the transduction. After 24 h, the medium was supplemented to bring the volume to 1 mL to facilitate cell growth.

### Co-Culture of Genetically Engineered TCR-T Cells and Allogeneic PBMC Cells

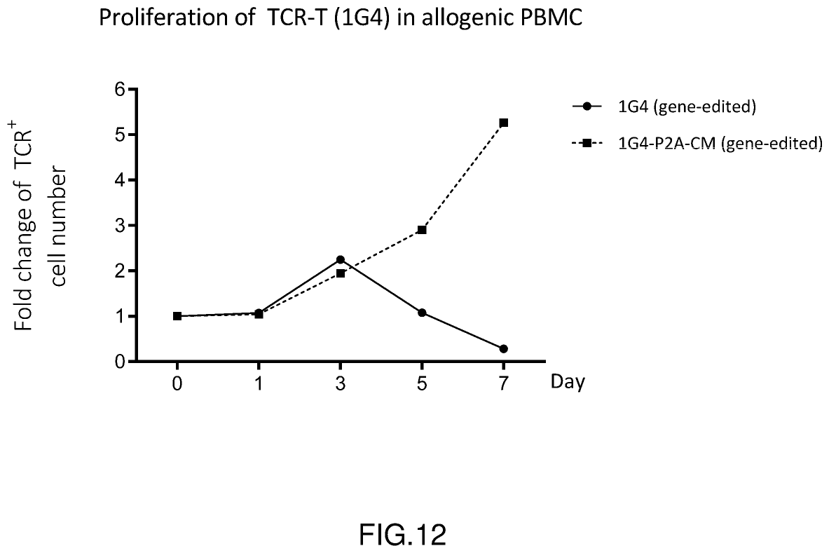

Experiment procedures were essentially the same as those described in Section 7.5 above. In FIG. 12, 1G4 cells were primary human T cells with both TRAC and β2M knocked out, and expressed the 1G4 TCR (SEQ ID NO:132); 1G4-P2A-CM cells were primary human T cells with both TRAC and β2M knocked out, and co-expressed 1G4 TCR and the fusion protein CM linked by a P2A peptide (SEQ ID NO:134). In order to measure the survival and growth of genetically engineered TCR-T cells (with or without CM) in the allogeneic PBMC environment, this study co-cultured three types of cells: 1G4 cells or 1G4-P2A-CM cells as effector cells; the K562 cells expressing single chain trimer (HLA-A0201 heavy chain, NY-ESO-1 derived peptide ‘SLLMWITQC’ (SEQ ID NO:140) and β2M), named K562-A02-NY-ESO-1, as the target cells; and the allogeneic PBMCs from peripheral blood of healthy human volunteers as a mimic of an allogeneic immune environment. The primary PBMCs and T cells used for preparing TCR-T or TCR-T-CM were obtained from different donors to elicit allogeneic responses. The cells were co-cultured according to the following ratio: TCR-positive cells in 1G4 or 1G4-P2A-CM cells: K562-A02-NY-ESO-1-tumor cells: allogeneic PBMCs=1:5:20 (cell number ratio). Allogeneic PBMCs and K562-A02-NY-ESO-1 cells were stained with Dye eFluor™ 670 prior to co-culture to be distinguished from the 1G4 cells or 1G4-P2A-CM cells. The cells were cultured at 37° C., and the numbers of HLA-ABC negative, TCR-positive cells in different groups were recorded at different time points.

FIG. 12 shows that in the presence of the allogeneic PBMC cells, 1G4 cells were not able to continuously expand with the stimulation of K562-A02-NY-ESO-1 cells. After a short time (3 days) of expansion, the number of 1G4 cells decreased quickly, whereas 1G4-P2A-CM cells were able to expand persistently throughout the co-culture period (7 days). These results indicated that immune cells in allogeneic PBMCs arrested the growth of 1G4 cells, but not the 1G4-P2A-CM cells, demonstrating that the fusion protein CM helped TCR-T cells to overcome growth inhibition and/or rejection by allogeneic immune cells, and to survive and grow in an allogeneic environment.

### 7.12 BCMA UCAR-T Cells Expressing Fusion Proteins were Resistant to Allogeneic NK Cells

### Preparation of T Cells Expressing BCMA CAR and Fusion Protein (CM)

Procedures described in sections 7.2 and 7.3 above were used to prepare the lentiviral constructs and genetically engineered T cells used in this study. First, a nucleic acid encoding BCMA CAR sequence (SEQ ID NO:136) linked to a nucleic acid sequence encoding fusion protein CM (SEQ ID NO:5) via P2A were synthesized and cloned into a lentiviral vector, named pLenti-BCMA-CM (BCMA CAR-CM: SEQ ID NO:138). CAR-T (without β2M/TRAC double knocked-out) and UCAR-T cells (with β2M/TRAC double knocked-out) were activated and transduced with pLenti-BCMA-CM. The resulting T cells were named BCMA-CM CAR-T and BCMA-CM UCAR-T respectively. Control T (without β2M/TRAC double knocked-out) and UT (with β2M/TRAC double knocked-out), both without lentiviral transduction, were included as control cells.

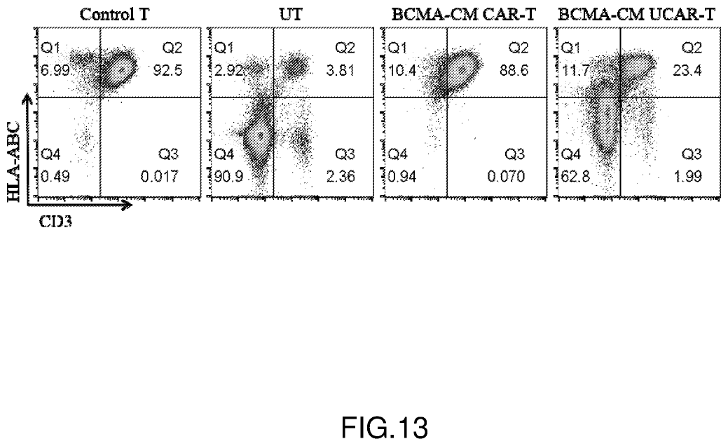

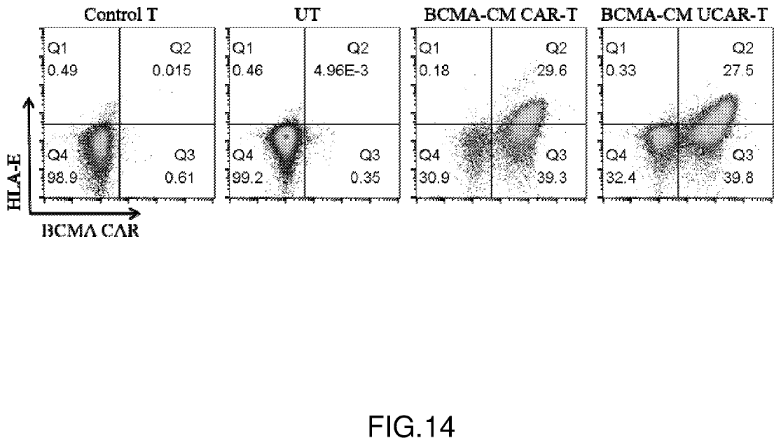

After T cell expansion, BCMA CAR and fusion protein CM expression levels were evaluated using fluorescence-labeled antibody staining and flow cytometry analysis according to procedures described in section 7.3 above. As shown in FIG. 13, HLA-ABC and CD3 expression were eliminated in UT and BCMA-CM UCAR-T cells. As shown in FIG. 14, BCMA CAR was expressed stably in all groups except in Control T and UT cells, and fusion protein CM was only expressed in BCMA-CM CAR-T and BCMA-CM UCAR-T cells as indicated by HLA-E expression, with CM expression at 29.6% and 27.5%, respectively.

### IFN-γ Release by BCMA-CM CAR-T and BCMA-CM UCAR-T Cells

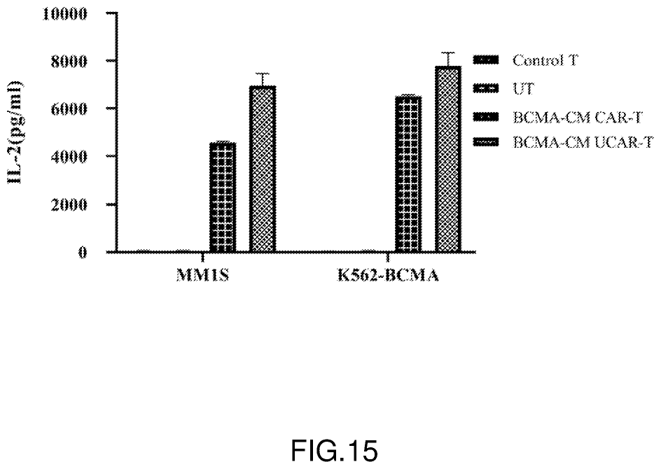

Target cells (MM1S.Luc cells and K562.BCMA.Luc cells) were each co-incubated with effector cells (Control T, UT, BCMA-CM CAR-T and BCMA-CM UCAR-T cells) for 20 hours at 1:1 ratio, respectively. The supernatant from the co-culture assays was collected to assess the content of interferon gamma (IFN-γ) using commercialized ELISA kit (Thermo Fisher Scientific; Catalog number: 88-7316). The assay was performed according to the protocol provided with the kit. As shown in FIG. 15, BCMA-CM CAR-T and BCMA-CM UCAR-T cells released high levels of IFN-γ upon co-culture with BCMA-expressing target cells.

BCMA UCAR-T Cells Expressing Fusion Proteins were Resistant to Allogeneic NK Cells

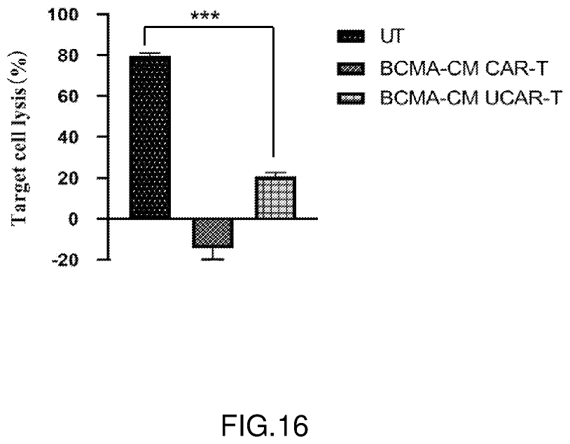

The NK-92 cytotoxicity assay was performed according to procedures described in section 7.4 above. Various groups of T cells (Control T, UT, BCMA-CM CAR-T and BCMA-CM UCAR-T cells) were co-incubated with NK-92 cells for 20 hours before flow cytometry analysis. As shown in FIG. 16, BCMA UCAR-T-CM cells showed a significantly higher level of resistance to NK cell-mediated cytotoxicity compared to UT cells without CM expression, which recapitulated previous results (note: the cell lysis rate was normalized to Control T cells; *** p<0.01).

### 8. REFERENCE TO SEQUENCE LISTING SUBMITTED ELECTRONICALLY

This application incorporates by reference a Sequence Listing which has been submitted electronically as an ASCII text file. The Sequence Listing is entitled “102A001WO02_SEQLIST_R.TXT,” created on Nov. 9, 2022 and has a size of 210,204 bytes.

1-100. (canceled)

101. A fusion protein comprising a presenting peptide covalently linked to a beta-2-microglobulin (β2M) peptide via a linker, wherein the presenting peptide is an HLA-E-restricted presenting peptide, or a signal peptide of a Class I MHC molecule or a fragment thereof, and wherein the fusion protein (1) comprises less than 500 amino acids, or (2) lacks an HLA-E heavy chain.

102. The fusion protein of claim 101, wherein the presenting peptide is derived from a virus, a prokaryote, a eukaryote, a mammal, or a human.

103. The fusion protein of claim 101, wherein the presenting peptide has 5-30 amino acids, 7-20 amino acids, or 8-10 amino acids.

104. The fusion protein of claim 101, wherein the presenting peptide is a signal peptide of a Class I MHC molecule or a fragment thereof.

105. The fusion protein of claim 104, wherein the Class I MHC molecule is selected from the group consisting of the heavy chains of HLA-A1, HLA-A2, HLA-A*3401, HLA-A*80, HLA-B7, HLA-B*13, HLA-B15, HLA-Cw3, HLA-Cw*2, HLA-Cw*0809, HLA-Cw7, HLA-Cw*1701, HLA-G, and HLA-F.

106. The fusion protein of claim 101, wherein the amino acid sequence of the presenting peptide comprises X₁X₂X₃X₄X₅X₆X₇X₈L; wherein X₁ is V or I; X₂ is T, A, M or L; X₃ is A, P, K or N; X₄ is P, L, or T; X₅ is R, Q or K; X₆ is T or A; X₇ is L, I, V or P; X₈ is V, L, I, F or T (SEQ ID NO:115).

107. The fusion protein of claim 106, wherein the amino acid sequence of the presenting peptide is selected from the group consisting of SEQ ID NOs: 21-73.

108. The fusion protein of claim 107, wherein the amino acid sequence of presenting peptide is VMAPRTVLL (SEQ ID NO: 38).

109. The fusion protein of claim 101, wherein the fusion protein has (1) less than 500, less than 400, less than 300, or less than 200 amino acids, or (2) about 120-180 amino acids.

110. The fusion protein of claim 101, comprising the presenting peptide, the linker, and the β2M peptide from N-terminus to C-terminus.

111. The fusion protein of claim 101, wherein the amino acid sequence of the linker comprises (1) (EAAAK)n, wherein n=3, 4 or 5 (SEQ ID NO: 110) or (2) (GGGGS)n, wherein n=3, 4 or 5 (SEQ ID NO: 112); and wherein the linker has between 5 and 30 amino acids.

112. The fusion protein of claim 111, wherein the amino acid sequence of the linker is SEQ ID NO:1 or 2.

113. The fusion protein of claim 101, wherein the amino acid sequence of the β2M peptide is at least 85%, at least 90%, at least 95%, or 100% identical to SEQ ID NO:81.

114. The fusion protein of claim 101 consisting of the presenting peptide, the linker, and the β2M peptide.

115. The fusion protein of claim 101, wherein the amino acid sequence of the fusion protein is at least 85%, at least 90%, at least 95%, or 100% identical to an amino acid sequence selected from the group consisting of SEQ ID NOs: 5, and 13-18.

116. A nucleic acid that encodes the fusion protein of claim 101.

117. The nucleic acid of claim 116, comprising (i) a first fragment encoding the fusion protein and (ii) a second fragment encoding a synthetic receptor.

118. The nucleic acid of claim 117, wherein the synthetic receptor is selected from the group consisting of a chimeric antigen receptor (CAR), a T cell receptor (TCR), a TCR receptor fusion construct (TRuC), a T cell antigen coupler (TAC), an antibody TCR receptor (AbTCR) and a chimeric CD3 receptor.

119. The nucleic acid of claim 117, wherein the synthetic receptor comprises an antigen-binding domain that specifically binds a tumor antigen or a viral antigen; wherein optionally the tumor antigen is selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1; and wherein optionally the viral antigen is EBV or HPV.

120. The nucleic acid of claim 118, wherein the synthetic receptor is a CAR having an antigen-binding domain that specifically binds CD19 or BCMA, wherein the amino acid sequence of the CAR is selected from the group consisting of SEQ ID NOs: 74-80 and 136; or wherein the synthetic receptor is a TCR having an antigen-binding domain that specifically binds NY-ESO-1, wherein the amino acid sequence of the TCR is SEQ ID NO:132.

121. The nucleic acid of claim 117, wherein the first and second fragments are connected via a polynucleotide encoding a 2A peptide; wherein optionally the 2A peptide is a P2A peptide, a T2A peptide, a F2A peptide, or an E2A peptide; or wherein the first and second fragments are connected via an IRES sequence.

122. A vector that comprises the nucleic acid of claim 116.

123. The vector of claim 122 that is a viral vector, wherein optionally the viral vector is a lentiviral vector, an adenoviral vector, or an adeno-associated viral vector.

124. A genetically engineered cell comprising the nucleic acid of claim 116.

125. The cell of claim 124, wherein the cell lacks endogenous expression of at least one gene encoding (1) a Class I MHC molecule selected from the group consisting of heavy chains of HLA-A, HLA-B, HLA-C, HLA-E, HLA-F, and HLA-G, (2) β2M, or (3) an MHC-like molecule selected from the group consisting of a CD1 heavy chain, or an MR1 heavy chain, an FcRn heavy chain, and UL18, on the cell surface.

126. The cell of claim 124, wherein the cell is an immune cell.

127. The immune cell of claim 126, wherein the immune cell is a leukocyte selected from the group consisting of a T cell, a NK cell, a NKT cell, a B cell, a plasma cell, a dendritic cell, a neutrophil, a monocyte, a macrophage, and a granulocyte.

128. The immune cell of claim 127, wherein the leukocyte is a T cell, and wherein optionally at least one gene encoding a component of TCR complex is inactivated in the T cell.

129. A pharmaceutical composition having the cell of claim 124 and a pharmaceutically acceptable excipient.

130. A method of treating a subject in need of an allogeneic transplant, comprising administering an effective amount of the cell of claim 124 to the subject.

131. A method of treating cancer in a subject comprising administering a therapeutically effective amount of the cell of claim 124 to the subject.

132. A method of genetically engineering a cell for an allogeneic transplant, comprising transducing the cell with the nucleic acid of claim 116.

133. The method of claim 132 further comprising inactivating at least one gene encoding (1) a Class I MHC molecule selected from the group consisting of heavy chains of HLA-A, HLA-B, HLA-C, HLA-E, HLA-F, and HLA-G, (2) β2M, or (3) an MHC-like molecule selected from the group consisting of a CD1 heavy chain, an MR1 heavy chain, an FcRn heavy chain and UL18.

134. The method of claim 133, wherein the gene is inactivated by DNA cleavage, DNA cleavage and repair, base editing, prime editing, RNA interference, RNA editing, or a CRISPR-Cas system.

135. A fusion protein comprising a presenting peptide covalently linked to a beta-2-microglobulin (β2M) peptide via a linker, wherein the fusion protein binds a major histocompatibility (MHC) heavy chain to form an MHC complex that binds an inhibitory receptor of an immune cell to inhibit the immune cell, and wherein the fusion protein (1) comprises less than 500 amino acids, or (2) lacks an HLA-E heavy chain.


SEQUENCE LISTING (140 sequences)

SEQ ID NO: 1 - protein, 15 aa, Artificial Sequence
  linker GGGGS
GGGGSGGGGSGGGGS

SEQ ID NO: 2 - protein, 20 aa, Artificial Sequence
  linker GGGGS
GGGGSGGGGSGGGGSGGGGS

SEQ ID NO: 3 - protein, 20 aa, Artificial Sequence
  signal peptide B2M
MSRSVALAVLALLSLSGLEA

SEQ ID NO: 4 - protein, 21 aa, Artificial Sequence
  signal peptide CD8
MALPVTALLLPLALLLHAARP

SEQ ID NO: 5 - protein, 143 aa, Artificial Sequence
  B7-B2M protein sequence
MSRSVALAVLALLSLSGLEAVMAPRTVLLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 6 - DNA, 432 bp, Artificial Sequence
  DNA coding sequence of SEQ ID NO. 5
atgtctcgctccgtggcattggctgtgctcgcgctactctctctttctggtctcgaagctgttatggctccgcggactgtgctgttaggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgtaa

SEQ ID NO: 7 - protein, 119 aa, Artificial Sequence
  full length B2M protein
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 8 - RNA, 109 nt, Artificial Sequence
  B2M sgRNA-1
cucacgcuggauagccuccguuuuagagcuaugcuggaaacagcauagcaaguuaaaauaaggcuaguccguuaucaacuugaaaaaguggcaccgagucggugcuuuu

SEQ ID NO: 9 - RNA, 109 nt, Artificial Sequence
  B2M sgRNA-2
aguagcgcgagcacagcuaguuuuagagcuaugcuggaaacagcauagcaaguuaaaauaaggcuaguccguuaucaacuugaaaaaguggcaccgagucggugcuuuu

SEQ ID NO: 10 - RNA, 99 nt, Artificial Sequence
  TRAC sgRNA
gagucucucagcugguacaguuuuagagcuagaaauagcaaguuaaaauaaggcuaguccguuaucaacuugaaaaaguggcaccgagucggugcuuuu

SEQ ID NO: 11 - protein, 659 aa, Artificial Sequence
  CAR19-P2A-B7-B2M protein
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHMRRKRGSGATNFSLLKQAGDVEENPGPMSRSVALAVLALLSLSGLEAVMAPRTVLLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 12 - DNA, 1980 bp, Artificial Sequence
  coding sequence for CAR19-P2A-B7-B2M protein
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccatatgcgccgcaagcgcggctccggtgccacgaacttctctctgttaaagcaagcaggagacgtggaagaaaaccccggtcctatgtctcgctccgtggcattggctgtgctcgcgctactctctctttctggtctcgaagctgttatggctccgcggactgtgctgttaggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgtaa

SEQ ID NO: 13 - protein, 143 aa, Artificial Sequence
  fusion protein
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 14 - protein, 143 aa, Artificial Sequence
  fusion protein
MSRSVALAVLALLSLSGLEAVMAPRTLVLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 15 - protein, 143 aa, Artificial Sequence
  fusion protein
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 16 - protein, 143 aa, Artificial Sequence
  fusion protein
MSRSVALAVLALLSLSGLEAVMAPRTLLLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 17 - protein, 143 aa, Artificial Sequence
  fusion protein
MSRSVALAVLALLSLSGLEAVTAPRTVLLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 18 - protein, 144 aa, Artificial Sequence
  fusion protein
MSRSVALAVLALLSLSGLEAYLLPRRGPRLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 19 - DNA, 1980 bp, Artificial Sequence
  DNA for coding CAR19-P2A-CM-c protein
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccatatgcgccgcaagcgcggctccggtgccacgaacttctctctgttaaagcaagcaggagacgtggaagaaaaccccggtcctatgtctcgctccgtggcattggctgtgctcgcgctactctctctttctggtctcgaagctatcattgacaagagcggctccaccgtgggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgtaa

SEQ ID NO: 20 - DNA, 1467 bp, Artificial Sequence
  DNA for coding CAR19 protein
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 21 - protein, 10 aa, Artificial Sequence
  P1 - examplery presenting peptide
YLLPRRGPRL

SEQ ID NO: 22 - protein, 9 aa, Artificial Sequence
  P2 - exemplary presenting peptide
ALALVRMLI

SEQ ID NO: 23 - protein, 9 aa, Artificial Sequence
  P3 - exemplary presenting peptide
AISPRTLNA

SEQ ID NO: 24 - protein, 9 aa, Artificial Sequence
  P4 - exemplary presenting peptide
QMRPVSRVL

SEQ ID NO: 25 - protein, 9 aa, Artificial Sequence
  P5 - exemplary presenting peptide
SQQPYLQLQ

SEQ ID NO: 26 - protein, 9 aa, Artificial Sequence
  P6 - exemplary presenting peptide
VTAPRTLLL

SEQ ID NO: 27 - protein, 9 aa, Artificial Sequence
  P7 - exemplary presenting peptide
VTAPRTVLL

SEQ ID NO: 28 - protein, 9 aa, Artificial Sequence
  P8 - exemplary presenting peptide
VTAPRTLVL

SEQ ID NO: 29 - protein, 9 aa, Artificial Sequence
  P9 - exemplary presenting peptide
VMAPQALLL

SEQ ID NO: 30 - protein, 9 aa, Artificial Sequence
  P10 - exemplary presenting peptide
VMAPRALLL

SEQ ID NO: 31 - protein, 9 aa, Artificial Sequence
  P11 - exemplary presenting peptide
VMAPRTLTL

SEQ ID NO: 32 - protein, 9 aa, Artificial Sequence
  P12 - exemplary presenting peptide
VMAPRTLFL

SEQ ID NO: 33 - protein, 9 aa, Artificial Sequence
  P13 - exemplary presenting peptide
VMAPRTLVL

SEQ ID NO: 34 - protein, 9 aa, Artificial Sequence
  P14 - exemplary presenting peptide
VMAPRTLIL

SEQ ID NO: 35 - protein, 9 aa, Artificial Sequence
  P15 - exemplary presenting peptide
IMAPRTLVL

SEQ ID NO: 36 - protein, 9 aa, Artificial Sequence
  P16 - exemplary presenting peptide
VMAPRTLLL

SEQ ID NO: 37 - protein, 9 aa, Artificial Sequence
  P17 - exemplary presenting peptide
VMPPRTLLL

SEQ ID NO: 38 - protein, 9 aa, Artificial Sequence
  P18 - exemplary presenting peptide
VMAPRTVLL

SEQ ID NO: 39 - protein, 9 aa, Artificial Sequence
  P19 - exemplary presenting peptide
VMAPRSLLL

SEQ ID NO: 40 - protein, 9 aa, Artificial Sequence
  P20 - exemplary presenting peptide
VMAPRSLIL

SEQ ID NO: 41 - protein, 9 aa, Artificial Sequence
  P21 - exemplary presenting peptide
VMTPRTLVL

SEQ ID NO: 42 - protein, 9 aa, Artificial Sequence
  P22 - exemplary presenting peptide
VMAPRILIL

SEQ ID NO: 43 - protein, 9 aa, Artificial Sequence
  P23 - exemplary presenting peptide
AMAPRTLIL

SEQ ID NO: 44 - protein, 9 aa, Artificial Sequence
  P24 - exemplary presenting peptide
VIAPRTLVL

SEQ ID NO: 45 - protein, 9 aa, Artificial Sequence
  P25 - exemplary presenting peptide
VMAPQSLLL

SEQ ID NO: 46 - protein, 9 aa, Artificial Sequence
  P26 - exemplary presenting peptide
VMAPRTFVL

SEQ ID NO: 47 - protein, 9 aa, Artificial Sequence
  P27 - exemplary presenting peptide
VMTPRTLIL

SEQ ID NO: 48 - protein, 9 aa, Artificial Sequence
  P28 - exemplary presenting peptide
VTAPRTLIL

SEQ ID NO: 49 - protein, 9 aa, Artificial Sequence
  P29 - exemplary presenting peptide
VMAPWTLLL

SEQ ID NO: 50 - protein, 9 aa, Artificial Sequence
  P30 - exemplary presenting peptide
VMVPRSLIL

SEQ ID NO: 51 - protein, 9 aa, Artificial Sequence
  P31 - exemplary presenting peptide
AMAPRTLVL

SEQ ID NO: 52 - protein, 9 aa, Artificial Sequence
  P32 - exemplary presenting peptide
VIAPRTLIL

SEQ ID NO: 53 - protein, 9 aa, Artificial Sequence
  P33 - exemplary presenting peptide
VIAPRTLLL

SEQ ID NO: 54 - protein, 9 aa, Artificial Sequence
  P34 - exemplary presenting peptide
VLAPRTLIL

SEQ ID NO: 55 - protein, 9 aa, Artificial Sequence
  P35 - exemplary presenting peptide
VMALRTLIL

SEQ ID NO: 56 - protein, 9 aa, Artificial Sequence
  P36 - exemplary presenting peptide
VMAPRGLIL

SEQ ID NO: 57 - protein, 9 aa, Artificial Sequence
  P37 - exemplary presenting peptide
VMAPRNLIL

SEQ ID NO: 58 - protein, 9 aa, Artificial Sequence
  P38 - exemplary presenting peptide
VMAPRTLFV

SEQ ID NO: 59 - protein, 9 aa, Artificial Sequence
  P39 - exemplary presenting peptide
VMAPRTLLM

SEQ ID NO: 60 - protein, 9 aa, Artificial Sequence
  P40 - exemplary presenting peptide
VMAPRTLVM

SEQ ID NO: 61 - protein, 9 aa, Artificial Sequence
  P41 - exemplary presenting peptide
VMAPRTSLL

SEQ ID NO: 62 - protein, 9 aa, Artificial Sequence
  P42 - exemplary presenting peptide
VMAPRTSVL

SEQ ID NO: 63 - protein, 9 aa, Artificial Sequence
  P43 - exemplary presenting peptide
VMAPWTLIL

SEQ ID NO: 64 - protein, 9 aa, Artificial Sequence
  P44 - exemplary presenting peptide
VMAPWTLVL

SEQ ID NO: 65 - protein, 9 aa, Artificial Sequence
  P45 - exemplary presenting peptide
VMDPRTLLL

SEQ ID NO: 66 - protein, 9 aa, Artificial Sequence
  P46 - exemplary presenting peptide
VMGPRTLIL

SEQ ID NO: 67 - protein, 9 aa, Artificial Sequence
  P47 - exemplary presenting peptide
VMGPRTLLL

SEQ ID NO: 68 - protein, 9 aa, Artificial Sequence
  P48 - exemplary presenting peptide
VMVPQTLIL

SEQ ID NO: 69 - protein, 9 aa, Artificial Sequence
  P49 - exemplary presenting peptide
VMVPRTLLL

SEQ ID NO: 70 - protein, 9 aa, Artificial Sequence
  P50 - exemplary presenting peptide
VVAPRTLIL

SEQ ID NO: 71 - protein, 9 aa, Artificial Sequence
  P51 - exemplary presenting peptide
VVAPRTLLL

SEQ ID NO: 72 - protein, 9 aa, Artificial Sequence
  P52 - exemplary presenting peptide
VMVPRTLIL

SEQ ID NO: 73 - protein, 9 aa, Artificial Sequence
  P53 - exemplary presenting peptide
VMATRTLLL

SEQ ID NO: 74 - protein, 488 aa, Artificial Sequence
  Anti-CD19 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 486 aa, Artificial Sequence
  Anti-CD19 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 489 aa, Artificial Sequence
  Anti-CD19 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYFWVLVVVGGVLACYSLLVTVAFIIFWVRSKRSRGGHSDYMNMTPRRPGPTRKHYQPYAPPRDFAAYRSVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 369 aa, Artificial Sequence
  Anti-BCMA CAR
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 78 - protein, 511 aa, Artificial Sequence
  Anti-BCMA CAR
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSGGGGSGGGGSAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTSTRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 496 aa, Artificial Sequence
  Anti-BCMA CAR
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTSTRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 493 aa, Artificial Sequence
  Anti-BCMA CAR
MALPVTALLLPLALLLHAARPDIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 99 aa, Artificial Sequence [sequence omitted]

SEQ ID NO: 82 - protein, 233 aa, Artificial Sequence
  human NKG2A
MDNQGVIYSDLNLPPNPKRQQRKPKGNKSSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 83 - protein, 348 aa, Artificial Sequence [sequence omitted]

SEQ ID NO: 84 - protein, 348 aa, Artificial Sequence [sequence omitted]

SEQ ID NO: 85 - protein, 341 aa, Artificial Sequence
  KIR2DL3
MSLMVVSMVCVGFFLLQGAWPHEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSETGNPRHLHVLIGTSVVIILFILLLFFLLHRWCCNKKNAVVMDQEPAGNRTVNREDSDEQDPQEVTYAQLNHCVFTQRKITRPSQRPKTPPTDIIVYTELPNAEP

SEQ ID NO: 86 - protein, 377 aa, Artificial Sequence [sequence omitted]

SEQ ID NO: 87 - protein, 375 aa, Artificial Sequence
  KIR2DL5
MSLMVVSMACVGFFLLQGAWTHEGGQDKPLLSAWPSAVVPRGGHVTLLCRSRLGFTIFSLYKEDGVPVPELYNKIFWKSILMGPVTPAHAGTYRCRGSHPRSPIEWSAPSNPLVIVVTGLFGKPSLSAQPGPTVRTGENVTLSCSSRSSFDMYHLSREGRAHEPRLPAVPSVNGTFQADFPLGPATHGGTYTCFGSLHDSPYEWSDPSDPLLVSVTGNSSSSSSSPTEPSSKTGIRRHLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 88 - protein, 444 aa, Artificial Sequence [sequence omitted]

SEQ ID NO: 89 - protein, 455 aa, Artificial Sequence
  KIR3DL2
MSLTVVSMACVGFFLLQGAWPLMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHREGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFHALPCVWSNSSDPLLVSVTGNPSSSWPSPTEPSSKSGICRHLHVLIGTSVVIFLFILLLFFLLYRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 90 - protein, 410 aa, Artificial Sequence [sequence omitted]

SEQ ID NO: 91 - protein, 650 aa, Artificial Sequence
  LIR1
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 92 - protein, 164 aa, Artificial Sequence
  CD3-zeta
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 93 - DNA, 339 bp, Artificial Sequence
  CD3-zeta
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 94 - protein, 220 aa, Artificial Sequence
  CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 95 - DNA, 321 bp, Artificial Sequence
  CD28
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 96 - protein, 255 aa, Artificial Sequence
  4-1BB
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 97 - protein, 277 aa, Artificial Sequence
  OX40
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 98 - protein, 199 aa, Artificial Sequence
  ICOS
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 99 - protein, 93 aa, Artificial Sequence
  DAP10
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 100 - protein, 260 aa, Artificial Sequence
  CD27
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 101 - protein, 595 aa, Artificial Sequence
  CD30
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 102 - protein, 277 aa, Artificial Sequence
  CD40
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 103 - protein, 235 aa, Artificial Sequence
  CD8
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 104 - protein, 458 aa, Artificial Sequence
  CD4
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 105 - protein, 22 aa, Artificial Sequence
  2A self-cleaving peptides - P2A
  MISC_FEATURE (1)..(3): GSG on the N-terminal of a 2A peptide is optional
GSGATNFSLLKQAGDVEENPGP

SEQ ID NO: 106 - protein, 21 aa, Artificial Sequence
  2A self-cleaving peptides - T2A
  MISC_FEATURE (1)..(3): GSG on the N-terminal of a 2A peptide is optional
GSGEGRGSLLTCGDVEENPGP

SEQ ID NO: 107 - protein, 23 aa, Artificial Sequence
  2A self-cleaving peptides - E2A
  MISC_FEATURE (1)..(3): GSG on the N-terminal of a 2A peptide is optional
GSGQCTNYALLKLAGDVESNPGP

SEQ ID NO: 108 - protein, 25 aa, Artificial Sequence
  2A self-cleaving peptides - F2A
  MISC_FEATURE (1)..(3): GSG on the N-terminal of a 2A peptide is optional
GSGVKQTLNFDLLKLAGDVESNPGP

SEQ ID NO: 109 - protein, 5 aa, Artificial Sequence
  linker sequence
  REPEAT (1)..(5): the 5 amino acids peptide can be repeated 1, 2, 3, 4, or 5 times
EAAAK

SEQ ID NO: 110 - protein, 5 aa, Artificial Sequence
  linker sequence
  REPEAT (1)..(5): the 5 amino acids peptide can be repeated 3, 4, or 5 times
EAAAK

SEQ ID NO: 111 - protein, 5 aa, Artificial Sequence
  linker sequence
  REPEAT (1)..(5): the 5 amino acids peptide can be repeated 1, 2, 3, 4, or 5 times
GGGGS

SEQ ID NO: 112 - protein, 5 aa, Artificial Sequence
  linker sequence
  REPEAT (1)..(5): the 5 amino acids peptide can be repeated 3, 4, or 5 times
GGGGS

SEQ ID NO: 113 - protein, 2 aa, Artificial Sequence
  linker sequence
  REPEAT (1)..(1): G can be repeated 1, 2, 3, 4, or 5 times, but not less than 1 time
GS

SEQ ID NO: 114 - protein, 14 aa, Artificial Sequence
  linker sequence
GGGGSGGSGSGGGS

SEQ ID NO: 115 - protein, 9 aa, Artificial Sequence
  consensus of signal peptide for fusion proteins
  MISC_FEATURE (1)..(1): X = Val or Ile
  MISC_FEATURE (2)..(2): X = Thr, Ala, Met or Leu
  MISC_FEATURE (3)..(3): X = Ala, Pro, Lys or Asn
  MISC_FEATURE (4)..(4): X = Pro, Leu or Thr
  MISC_FEATURE (5)..(5): X = Arg, Gln or Lys
  MISC_FEATURE (6)..(6): X = Thr or Ala
  MISC_FEATURE (7)..(7): X = Leu, Ile, Val or Pro
  MISC_FEATURE (8)..(8): X = Val, Leu, Ile, Phe or Thr
XXXXXXXXL

SEQ ID NO: 116 - protein, 21 aa, Artificial Sequence
  CD8
MALPVTALLLPLALLLHAARP

SEQ ID NO: 117 - protein, 9 aa, Artificial Sequence
  HLA-C restricted presenting peptide
IIDKSGSTV

SEQ ID NO: 118 - protein, 113 aa, Artificial Sequence
  DAP12
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 119 - protein, 216 aa, Artificial Sequence
  NKG2D
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 120 - protein, 567 aa, Artificial Sequence
  Megf10
MVISLNSCLSFICLLLCHWIGTASPLNLEDPNVCSHWESYSVTVQESYPHPFDQIYYTSCTDILNWFKCTRHRVSYRTAYRHGEKTMYRRKSQCCPGFYESGEMCVPHCADKCVHGRCIAPNTCQCEPGWGGTNCSSACDGDHWGPHCTSRCQCKNGALCNPITGACHCAAGFRGWRCEDRCEQGTYGNDCHQRCQCQNGATCDHVTGECRCPPGYTGAFCEDLCPPGKHGPQCEQRCPCQNGGVCHHVTGECSCPSGWMGTVCGQPCPEGRFGKNCSQECQCHNGGTCDAATGQCHCSPGYTGERCQDECPVGTYGVLCAETCQCVNGGKCYHVSGACLCEAGFAGERCEARLCPEGLYGIKCDKRCPCHLENTHSCHPMSGECACKPGWSGLYCNETCSPGFYGEACQQICSCQNGADCDSVTGKCTCAPGFKGIDCSTPCPLGTYGINCSSRCGCKNDAVCSPVDGSCTCKAGWHGVDCSIRCPSGTWGFGCNLTCQCLNGGACNTLDGTCTCAPGWRGEKCELPCQDGTYGLNCAERCDCSHADGCHPTTGHCRCLPGWSGLF

SEQ ID NO: 121 - protein, 86 aa, Artificial Sequence
  FcR-gamma
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 122 - protein, 2 aa, artificial sequence
  linker sequence
  REPEAT (1)..(2): (GnS)m; n=1, 2, 3, 4, and 5; m=1, 2, 3, 4, or 5; In (GnS), G can be repeated 1, 2, 3, 4, or 5 times, but not less than 1 time; (GnS) can be repeated 1, 2, 3, 4, or 5 times, but not less than 1 time.
GS

SEQ ID NO: 123 - DNA, 1461 bp, Artificial Sequence
  CJP CAR19 DNA sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 124 - protein, 656 aa, artificial sequence
  CJP-P2A-CM amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRRKRGSGATNFSLLKQAGDVEENPGPMSRSVALAVLALLSLSGLEAVMAPRTVLLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 125 - DNA, 1971 bp, Artificial Sequence
  CJP-P2A-CM DNA sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggccgccgcaagcgcggctccggtgccacgaacttctctctgttaaagcaagcaggagacgtggaagaaaaccccggtcctatgtctcgctccgtggcattggctgtgctcgcgctactctctctttctggtctcgaagctgttatggctccgcggactgtgctgttaggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgtaa

SEQ ID NO: 126 - protein, 656 aa, artificial sequence
  CJP-P2A-CM12 amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRRKRGSGATNFSLLKQAGDVEENPGPMSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 127 - DNA, 1971 bp, artificial sequence
  CJP-P2A-CM12 DNA sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggccgccgcaagcgcggctccggtgccacgaacttctctctgttaaagcaagcaggagacgtggaagaaaaccccggtcctatgtctcgctccgtggcattggctgtgctcgcgctactctctctttctggtctcgaagctgttatggctccgcggactctgttcttaggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgtaa

SEQ ID NO: 128 - protein, 656 aa, artificial sequence
  CJP-P2A-CM13 amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRRKRGSGATNFSLLKQAGDVEENPGPMSRSVALAVLALLSLSGLEAVMAPRTLVLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 129 - DNA, 1971 bp, artificial sequence
  CJP-P2A-CM13 DNA sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggccgccgcaagcgcggctccggtgccacgaacttctctctgttaaagcaagcaggagacgtggaagaaaaccccggtcctatgtctcgctccgtggcattggctgtgctcgcgctactctctctttctggtctcgaagctgttatggctccgcggactctggtgttaggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgtaa

SEQ ID NO: 130 - protein, 656 aa, Artificial sequence
  CJP-P2A-CM14 amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRRKRGSGATNFSLLKQAGDVEENPGPMSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 131 - DNA, 1971 bp, Artificial sequence
  CJP-P2A-CM14 DNA Sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggccgccgcaagcgcggctccggtgccacgaacttctctctgttaaagcaagcaggagacgtggaagaaaaccccggtcctatgtctcgctccgtggcattggctgtgctcgcgctactctctctttctggtctcgaagctgttatggctccgcggactctgatcttaggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgtaa

SEQ ID NO: 132 - protein, 611 aa, Artificial sequence
  1G4-TCR amino acid sequence
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGEGRGSLLTCGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 133 - DNA, 1836 bp, artificial sequence
  1G4-TCR DNA sequence
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagccgggccaagcggtccggctccggagagggcaggggatctctccttacttgtggcgacgtggaggagaaccccggccccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaagaacgtgttcccccccgaggtggccgtgttcgagcccagcgaggccgagatcagccacacccagaaagccaccctggtgtgcctggccaccggcttctaccccgatcacgtggagctgtcttggtgggtgaacggcaaagaggtgcactccggcgtctgcaccgaccctcagcccctgaaagagcagcccgccctgaacgacagccggtactgcctgtcctcccggctgagagtgtctgctacattctggcagaatccccggaaccacttccggtgccaggtgcagttctacggcctgagcgagaacgacgagtggacccaggacagagccaagcccgtgacccagatcgtgtccgccgaggcctggggcagagccgactgcggcttcaccagcgagagctaccagcagggcgtgctgtctgccaccatcctgtacgagatcctgctgggcaaggccaccctgtacgccgtgctggtgtccgccctggtgctgatggccatggtgaagcggaaggacagcagaggctga

SEQ ID NO: 134 - protein, 780 aa, artificial sequence
  1G4-P2A-CM  Amino acid sequence
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGEGRGSLLTCGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRRKRGSGATNFSLLKQAGDVEENPGPMSRSVALAVLALLSLSGLEAVMAPRTVLLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 135 - DNA, 2343 bp, Artificial sequence
  1G4-P2A-CM DNA sequence
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctgtacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagccgggccaagcggtccggctccggagagggcaggggatctctccttacttgtggcgacgtggaggagaaccccggccccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaagaacgtgttcccccccgaggtggccgtgttcgagcccagcgaggccgagatcagccacacccagaaagccaccctggtgtgcctggccaccggcttctaccccgatcacgtggagctgtcttggtgggtgaacggcaaagaggtgcactccggcgtctgcaccgaccctcagcccctgaaagagcagcccgccctgaacgacagccggtactgcctgtcctcccggctgagagtgtctgctacattctggcagaatccccggaaccacttccggtgccaggtgcagttctacggcctgagcgagaacgacgagtggacccaggacagagccaagcccgtgacccagatcgtgtccgccgaggcctggggcagagccgactgcggcttcaccagcgagagctaccagcagggcgtgctgtctgccaccatcctgtacgagatcctgctgggcaaggccaccctgtacgccgtgctggtgtccgccctggtgctgatggccatggtgaagcggaaggacagcagaggccgccgcaagcgcggctccggtgccacgaacttctctctgttaaagcaagcaggagacgtggaagaaaaccccggtcctatgtctcgctccgtggcattggctgtgctcgcgctactctctctttctggtctcgaagctgttatggctccgcggactgtgctgttaggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgtaa

SEQ ID NO: 136 - protein, 373 aa, artificial sequence
  BCMA CAR amino acid sequence
MALPVTALLLPLALLLHAARPQVQLVESGGGLVQAGGSLRLSCAASGLTFSEYTMGWFRQPPGKEREFVAAISGGGIPYYRTTVKGRFSISRDNAQNTVALEMNSLKPEDTAVYYCAADARAVMTVTPNYWGQGTQVTVSSEPKTPKPQDTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - DNA, 1119 bp, artificial sequence
  BCMA CAR DNA sequence
atggccttaccagtgaccgccttgctcctgccgctggccttgctgctccacgccgccaggccgcaggtgcagctcgtggagtctgggggaggattggtgcaggctgggggctcgctcagactctcctgcgcagcctctggacttaccttcagtgaatataccatgggctggttccgccagcctccagggaaggagcgtgaatttgtagcggctattagtggaggtggtatcccatactatagaaccaccgtgaagggtcgattctccatctccagagacaacgcccagaacacggtcgctctggagatgaacagcctgaaacctgaggacacggccgtttattactgtgcagcagatgcgcgtgcggtgatgactgtgactcccaactactggggccaggggacccaggtcaccgtctcctcagaacccaagacaccaaaaccacaagacaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 138 - protein, 543 aa, artificial sequence
  BCMA CAR-CM amino acid sequence
MALPVTALLLPLALLLHAARPQVQLVESGGGLVQAGGSLRLSCAASGLTFSEYTMGWFRQPPGKEREFVAAISGGGIPYYRTTVKGRFSISRDNAQNTVALEMNSLKPEDTAVYYCAADARAVMTVTPNYWGQGTQVTVSSEPKTPKPQDTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRRKRGSGATNFSLLKQAGDVEENPGPMSRSVALAVLALLSLSGLEAVMAPRTVLLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 139 - DNA, 1629 bp, artificial sequence
  BCMA CAR-CM DNA sequence
atggccttaccagtgaccgccttgctcctgccgctggccttgctgctccacgccgccaggccgcaggtgcagctcgtggagtctgggggaggattggtgcaggctgggggctcgctcagactctcctgcgcagcctctggacttaccttcagtgaatataccatgggctggttccgccagcctccagggaaggagcgtgaatttgtagcggctattagtggaggtggtatcccatactatagaaccaccgtgaagggtcgattctccatctccagagacaacgcccagaacacggtcgctctggagatgaacagcctgaaacctgaggacacggccgtttattactgtgcagcagatgcgcgtgcggtgatgactgtgactcccaactactggggccaggggacccaggtcaccgtctcctcagaacccaagacaccaaaaccacaagacaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggccgccgcaagcgcggctccggtgccacgaacttctctctgttaaagcaagcaggagacgtggaagaaaaccccggtcctatgtctcgctccgtggcattggctgtgctcgcgctactctctctttctggtctcgaagctgttatggctccgcggactgtgctgttaggtggtggcggttccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatg

SEQ ID NO: 140 - protein, 9 aa, artificial sequence
  NY-ESO-1 derived peptide
SLLMWITQC